(19) United States
(12) Patent Application Publication
(10) Pub. No.: US 2024/0361321 A1
(43) Pub. Date: Oct. 31, 2024

(54) LUMINESCENT BASED ANTIGEN ASSAY

(71) Applicants: INSTITUT PASTEUR, PARIS (FR); CENTRE NATIONAL DE LA RECHERCHE SCIENTIFIQUE, PARIS (FR)

(72) Inventors: Thierry ROSE, PARIS (FR); Sophie GOYARD, PARIS (FR); Yves JANIN, PARIS (FR); Pierre LAFAYE, PARIS (FR); Gabriel AYME, PARIS (FR); Nicolas ESCRIOU, PARIS (FR); Marion GRANSAGNE, PARIS (FR)

(21) Appl. No.: 18/685,111

(22) PCT Filed: Aug. 23, 2022
(86) PCT No.: PCT/EP2022/073507

§ 371 Date: Feb. 20, 2024

(30) Foreign Application Priority Data
Aug. 24, 2021 (EP) 21306138.5

(51) Int. Cl. G01N 33/569; C07K 16/10; C12Q 1/66; G01N 33/68

(52) U.S. Cl. CPC G01N 33/56988; C07K 16/1003; C07K 16/1054; C12Q 1/66; G01N 33/6854; C07K 2317/22; C07K 2317/569; C07K 2319/61; G01N 2333/161; G01N 2333/165; G01N 2333/90241

(57) ABSTRACT

System for detecting an antigen comprising: —a first fusion protein with no luciferase activity comprising: —a N-terminal domain which comprises a first single domain antibody which is directed against a first epitope of said antigen and —a C-terminal domain which comprises a first fragment of a luciferase having the amino acid sequence SEQ ID NO: 1 or is a variant thereof and —a second fusion protein with no luciferase activity comprising: —a N-terminal domain which comprises a second single domain antibody which is directed against a second epitope of said antigen and —a C-terminal domain which comprises a second fragment of a luciferase having the amino acid sequence SEQ ID NO: 2 or is a variant thereof. Luminescence is emitted in the presence of a substrate when both the first fusion protein and the second fusion protein bind to said antigen.

### FIELD OF THE INVENTION

The present invention relates to fusion proteins, system, kit comprising thereof, and method for quantitative detection of a soluble or surface-bound antigen with instant results capabilities for prognosis, diagnosis and therapy follow-up purposes.

### BACKGROUND OF THE INVENTION

Many in vitro assays for diagnosing infectious diseases or cancer are available. These assays include notably nucleic acid amplification tests, serologic and antigen-based assays. Choice of the most appropriate diagnosing assay among all the one available depends on many criteria such as timing relative to disease course, individual or collective diagnosis, laboratory infrastructure etc.

For rapid identification of infectious, inflammation or cancer cases in disease course the antigen-based assay is the most indicated. Antigen-based diagnostics usually detect protein fragments on or within an infectious agent or a tumor cell. Standard antigen assays use two main approaches: 1) the immuno-chromatographic or lateral flow assay based either on colloid gold conjugated antibodies that result in visible colored bands to reflect positivity or on fluorescence conjugate antibodies that provides results via an automated immunofluorescence reader, 2) the enzyme-linked immunosorbent assays (ELISA) based on a sandwich of antibodies, one coating the plate well surface, the second labelled with an enzyme (peroxidase, phosphatase or luciferase) capturing soluble antigens revealed in the presence of enzyme substrates detected by light absorption, fluorescence or light emission. These assays have usually a good specificity, the first is rapid but mostly qualitative (5-30 min), the second is longer (30 min-3 h) but sensitive and quantitative. The sensitivity is often dependent on the infectious load and the volume of sample. Moreover, these assays are also highly dependent on the quality of the sample in particular its storage conditions and don't provide instant results but require more than 15 min. There is also need for extension of the uses of such tests beyond body fluids, to cell or tissue lysates or extracts for human or animal health care but also for food industry, environment and sewage survey. Further developments are therefore needed to improve and ease the current antigen-based assays.

### SUMMARY OF THE INVENTION

Now, the applicant has found bioluminescence-based method for qualitative and/or quantitative detection of an antigen with instant results capabilities and easy use with no coating step, no washes and no incubation time. The inventors have optimized luciferase(s) derived from the KAZ (Inouye, S., Sato, J., Sahara-Miura, Y., Yoshida, S. and Hosoya, T., Luminescence enhancement of the catalytic 19 kDa protein (KAZ) of Oplophorous luciferase by three amino acid substitutions. Biochem. Biophys. Res. Commun. 2014. 445:157-162) or Nluc (Hall, M. P., Unch, J., Binkowski, B. F., Valley, M. P., Butler, B. L., Wood, M. G., Otto, P., Zimmerman, K., Vidugiris, G., Machleidt, T., Robers, M. B., Benink, H. A., Eggers, C. T., Slater, M. R., Meisenheimer, P. L., Klaubert, D. H., Fan, F., Encell, L. P., and Wood, K. V. 2012 Engineered luciferase reporter from a deep sea shrimp utilizing a novel imidazopyrazinone substrate ACS Chem. Biol. 7, 1848-1857) that has been shortened and engineered from the Oplophorous gracilirostris native catalytic enzyme subunit. The optimized luciferase was divided into two inactive fragments and each one of these fragments was fused preferably by a linker to one variable domain of a camelid heavy-chain antibody (VHH) directed against an antigen. The luciferase activity was restored when the two fusion proteins bound to their respective epitope on the antigen. Using this finding, the inventors have developed a new quick antigen assay which does not require coating, washing or incubation time and can provide instant results (<1 min). Moreover, the antigen assay developed by the inventors is usable on most biological samples (body fluids, rhino-pharyngeal swab wash, organ wash, faeces or skin smears, cell or tissue lysate or extract, cell culture media or supernatant, etc . . . ), environment fluid or surface smear, water or sewage sample, food ingredient extract or smear, drugs etc . . . and the assay reagents can be stored at 4° C. for weeks, −20° C. for months and −80° C. for years.

A subject of the present invention is therefore a system for detecting an antigen comprising:
a first fusion protein comprising:
   a N-terminal domain which comprises a first single domain antibody which is directed against a first epitope of said antigen
      and
a C-terminal domain which comprises a first fragment of a luciferase:
wherein the first fragment has:
the amino acid sequence as set forth in SEQ ID NO: 1 or
an amino acid sequence having at least 70% amino acid sequence identity to the amino acid sequence as set forth in SEQ ID NO: 1,
   wherein the first fusion protein has no luciferase activity,
   and
a second fusion protein comprising:
   a N-terminal domain which comprises a second single domain antibody which is directed against a second epitope of said antigen
      and
a C-terminal domain which comprises a second fragment of a luciferase:
wherein the second fragment has:
the amino acid sequence as set forth in SEQ ID NO: 2 or
an amino acid sequence having at least 70% amino acid sequence identity to the amino acid sequence as set forth in SEQ ID NO: 2,
   wherein the second fusion protein has no luciferase activity,
   luminescence being emitted in the presence of a substrate when both the first fusion protein and the second fusion protein bind to said antigen.

### DETAILED DESCRIPTION OF THE INVENTION

### A. Fusion Proteins

A subject matter of the present invention relates to a fusion protein comprising:
a N-terminal domain which comprises a single domain antibody, preferably a variable domain of a camelid heavy-chain antibody (VHH), which is directed against an epitope of an antigen
   and
a C-terminal domain which comprises a fragment of a luciferase:
wherein the fragment has:
   the amino acid sequence as set forth in SEQ ID NO: 1 or an amino acid sequence having at least 70%, at least 75%, at least 80%, at least 85%, at least 90%, at least 95%, at least 97% or at least 99% amino acid sequence identity to the amino acid sequence as set forth in SEQ ID NO: 1
or
the amino acid sequence as set forth in SEQ ID NO: 2 or an amino acid sequence having at least 70%, at least 75%, at least 80%, at least 85%, at least 90%, at least 95%, at least 97% or at least 99% amino acid sequence identity to the amino acid sequence as set forth in SEQ ID NO: 2.

The fusion protein has no luciferase activity.

The presence/absence of a luciferase activity can easily be assayed by a person skilled in the art. The luciferase activity of the fusion protein may be for example assayed with 8-(2,3-difluorobenzyl)-2-((5-methylfuran-2-yl)methyl)-6-phenylimidazo[1,2-a]pyrazin-3(7H)-one as substrate, a blank control and a positive control for example with the luciferase having the amino acid sequence SEQ ID NO: 3. The following percentage of relative luciferase activity may be calculated: [luminescence of the fusion protein−luminescence of the blank control]×100/luminescence of the positive control. If this percentage is negative, null or non-significant (e. g. lower than 10%, preferably than 5%, more preferably lower than 2.5%, most preferably lower than 1%), the person skilled in the art will consider that the fusion protein has no luciferase activity.

The C-terminal domain may further comprise an heterologous sequence such as for example a signal peptide and/or a tag.

The fusion protein may further comprise a linker between the N-terminal and the C-terminal domains.

The C-terminal domain of the fusion protein may consist of a fragment of a luciferase:
wherein the fragment has:
   the amino acid sequence as set forth in SEQ ID NO: 1 or an amino acid sequence having at least 70%, at least 75%, at least 80%, at least 85%, at least 90%, at least 95%, at least 97% or at least 99% amino acid sequence identity to the amino acid sequence as set forth in SEQ ID NO: 1
or
the amino acid sequence as set forth in SEQ ID NO: 2 or an amino acid sequence having at least 70%, at least 75%, at least 80%, at least 85%, at least 90%, at least 95%, at least 97% or at least 99% amino acid sequence identity to the amino acid sequence as set forth in SEQ ID NO: 2,
   optionally linked to an heterologous sequence such as for example a signal peptide and/or a tag.

The fusion protein may consists of:
a N-terminal domain which consists of a single domain antibody, preferably a variable domain of a camelid heavy-chain antibody (VHH), which is directed against an epitope of an antigen, the single domain antibody being optionally linked to an heterologous sequence,
a C-terminal domain which consists of a fragment of a luciferase wherein the fragment has:
the amino acid sequence as set forth in SEQ ID NO: 1 or an amino acid sequence having at least 70%, at least 75%, at least 80%, at least 85%, at least 90%, at least 95%, at least 97% or at least 99% amino acid sequence identity to the amino acid sequence as set forth in SEQ ID NO: 1 or
the amino acid sequence as set forth in SEQ ID NO: 2 or an amino acid sequence having at least 70%, at least 75%, at least 80%, at least 85%, at least 90%, at least 95%, at least 97% or at least 99% amino acid sequence identity to the amino acid sequence as set forth in SEQ ID NO: 2,
   the fragment of a luciferase being optionally linked to an heterologous sequence such as for example a signal peptide and/or a tag,
and a linker between the N-terminal and the C-terminal domains.

In an embodiment, the fusion protein, called the first fusion protein, comprises:
a N-terminal domain which comprises a single domain antibody, called first single domain antibody, preferably a VHH called first VHH, which is directed against a first epitope of the antigen
   and
a C-terminal domain which comprises a fragment, called first fragment, of a luciferase:
wherein the first fragment has:
   the amino acid sequence as set forth in SEQ ID NO: 1 or
an amino acid sequence having at least 70%, at least 75%, at least 80%, at least 85%, at least 90%, at least 95%, at least 97% or at least 99% amino acid sequence identity to the amino acid sequence as set forth in SEQ ID NO: 1.

The first fusion protein has no luciferase activity.

The first fusion protein specifically binds the antigen.

The C-terminal domain of the first fusion protein may further comprise an heterologous sequence such as for example a signal peptide and/or a tag.

The first fusion protein may further comprise a linker between the N-terminal and the C-terminal domains.

An antigen binding protein (in the context of the invention the single domain antibody, the VHH, the (first and/or second) fusion protein) is said to “specifically bind” its target antigen when the dissociation constant (K_{D}) is ≤10⁻⁷ M. The antigen binding protein specifically binds antigen with “high affinity” when the K_{D} is ≤5×10⁻⁹ M, and with “very high affinity” when the K_{D} is ≤5×10⁻¹⁰ M.

In one embodiment, the first fusion protein binds the antigen with a K_{D}≤10⁻⁷ M, preferably between about 10⁻⁹ M and 10⁻¹³ M.

The first fusion protein binds specifically the first epitope of the antigen.

The term “epitope” includes any determinant capable being bound by an antigen binding protein, such as an antibody, a T-cell receptor or in a context of the invention a VHH or a fusion protein. An epitope is a region of an antigen that is bound by an antigen binding protein that targets that antigen, and when the antigen is a protein, includes specific amino acids that directly contact the antigen binding protein. Most often, epitopes reside on proteins, but in some instances can reside on other kinds of molecules, such as nucleic acids. Epitope determinants can include chemically active surface groupings of molecules such as amino acids, sugar side chains, phosphoryl or sulfonyl groups, and can have specific three-dimensional structural characteristics, and/or specific charge characteristics. Generally, antibodies specific for a particular target antigen will preferentially recognize an epitope on the target antigen in a complex mixture of proteins and/or macromolecules.

The C-terminal domain of the first fusion protein may consist of a fragment of a luciferase:
wherein the fragment has:
   the amino acid sequence as set forth in SEQ ID NO: 1 or an amino acid sequence having at least 70%, at least 75%, at least 80%, at least 85%, at least 90%, at least 95%, at least 97% or at least 99% amino acid sequence identity to the amino acid sequence as set forth in SEQ ID NO: 1,
      optionally linked to an heterologous sequence such as for example a signal peptide and/or a tag.

The first fusion protein may consist of:
a N-terminal domain which comprises a single domain antibody called first single domain antibody, preferably a VHH called first VHH, which is directed against a first epitope of the antigen, the single domain antibody being optionally linked to an heterologous sequence,
a C-terminal domain which consists of a first fragment of a luciferase wherein the first fragment has the amino acid sequence as set forth in SEQ ID NO: 1 or an amino acid sequence having at least 70%, at least 75%, at least 80%, at least 85%, at least 90%, at least 95%, at least 97% or at least 99% amino acid sequence identity to the amino acid sequence as set forth in SEQ ID NO: 1, the fragment of a luciferase being optionally linked to an heterologous sequence such as for example a signal peptide and/or a tag,
and a linker between the N-terminal and the C-terminal domains.

In an embodiment, the fusion protein, called the second fusion protein, comprises:
a N-terminal domain which comprises a single domain antibody called second single domain antibody, preferably a VHH called second VHH, which is directed against a second epitope of the antigen
   and
a C-terminal domain which comprises a fragment, called second fragment, of a luciferase:
wherein the second fragment has:
   the amino acid sequence as set forth in SEQ ID NO: 2 or
an amino acid sequence having at least 70%, at least 75%, at least 80%, at least 85%, at least 90%, at least 95%, at least 97% or at least 99% amino acid sequence identity to the amino acid sequence as set forth in SEQ ID NO: 2.

The second fusion protein has no luciferase activity.

The second fusion protein specifically binds the antigen.

In one embodiment, the second fusion protein binds the antigen with a K_{D}≤10⁻⁷ M, preferably between about 10⁻⁹ M and 10⁻¹³ M, in yet another embodiment a K_{D}≤5×10⁻¹⁰ M.

The second fusion protein binds specifically the second epitope of the antigen.

The C-terminal domain of the second fusion protein may further comprise an heterologous sequence such as for example a signal peptide and/or a tag.

The second fusion protein may further comprise a linker between the N-terminal and the C-terminal domains.

The C-terminal domain of the second fusion protein may consist of a fragment of a luciferase:
wherein the fragment has:
the amino acid sequence as set forth in SEQ ID NO: 2 or an amino acid sequence having at least 70%, at least 75%, at least 80%, at least 85%, at least 90%, at least 95%, at least 97% or at least 99% amino acid sequence identity to the amino acid sequence as set forth in SEQ ID NO: 2,
   optionally linked to an heterologous sequence such as for example a signal peptide and/or a tag.

The second fusion protein may consist of:
a N-terminal domain which comprises a single domain antibody called second single domain antibody, preferably a VHH called second VHH, which is directed against a second epitope of the antigen, the single domain antibody being optionally linked to an heterologous sequence,
a C-terminal domain which consists of a second fragment of a luciferase wherein the second fragment has the amino acid sequence as set forth in SEQ ID NO: 2 or an amino acid sequence having at least 70%, at least 75%, at least 80%, at least 85%, at least 90%, at least 95%, at least 97% or at least 99% amino acid sequence identity to the amino acid sequence as set forth in SEQ ID NO: 2, the second fragment of a luciferase being optionally linked to an heterologous sequence such as for example a signal peptide and/or a tag,
and a linker between the N-terminal and the C-terminal domains.

### A.1. Antigen

The fusion proteins of the invention aim to detect an antigen and possibly quantify its concentration.

An antigen is any specific molecule or molecule assembly recognisable by an antibody or a molecule binder. An antigen is either a protein, a nucleic acid, a polysaccharide, a lipid, an organic molecule or a covalent or non-covalent assembly of these identical or different compounds. Proteins can be biologically or chemically modified (glycosylation, acylation, phosphorylation, sulfonation, deamination, etc . . . ) or not. Nucleic acids can be RNA or DNA, single or double strand and chemically or biologically modified or not. The antigen can be soluble, solubilized from a cell lysate or tissue extract or presented at the surface of an organelle, a virus, a bacterium, a cell, a tissue, etc. The antigen can be exposed at the surface of any material composing a bead, a fibre, a slide, a stick, a disk, a tube, a plate well, a bag or any recipient.

The antigen may be from any pathogen, inflammatory or tumour cell, that is to be detected for presence in a sample. The pathogen may be for example selected from the group consisting of a phage, a virus, a bacterium, a yeast, a fungus and a parasite. Thus, the antigen may be any fragment or part of said pathogen. Fragment of a pathogen may comprise an isolated protein from the pathogen, synthesized or expressed as recombinant, or fragments corresponding to structural or functional domains or fragment of any size.

In a preferred embodiment of the invention, the pathogen whose presence is to be diagnosed is a virus, more preferably a coronavirus, most preferably a coronavirus selected from the group consisting of severe acute respiratory syndrome coronavirus 2 (SARS-CoV-2), severe acute respiratory syndrome coronavirus 1 (SARS-CoV-1) or Middle East respiratory syndrome-related coronavirus (MERS).

Coronaviruses are enveloped viruses with a positive RNA genome, belonging to the Coronaviridae family of the order Nidovirales, which are divided into four genera (α, β, γ, and δ). The SARS-CoV-2 as well as the SARS-CoV-1 and the MERS belongs to the β genus. Coronaviruses contain at least four structural proteins: Spike (S) protein, envelope (E) protein, membrane (M) protein, and nucleocapsid (N) protein (also called nucleoprotein) (Bosch B. J., van der Zee R., de Haan C. A., Rottier P. J. The coronavirus spike protein is a class I virus fusion protein: structural and functional characterization of the fusion core complex. J Virol. 2003; 77:8801-8811).

Because of their strong immunogenicity and their high expression level in infected cells, the N and S proteins of coronavirus are usually chosen as targets for diagnostic purpose. The coronavirus N protein is a homodimer formed by 2 monomers of 40 kDa. Each monomer is organized into two folded domains that are called the N-terminal domain (NTD) and the C-terminal domain (CTD). They are separated by a disordered region (called LKR) containing a serine/arginine stretch which could regulate the functions of N upon phosphorylation (McBride, R., van Zyl, M. & Fielding, B. C. The coronavirus nucleocapsid is a multifunctional protein. Viruses (2014) doi:10.3390, He, R. et al. Characterization of protein-protein interactions between the nucleocapsid protein and membrane protein of the SARS coronavirus. Virus Res. (2004) doi:10.1016/j.virusres.2004.05.002.). Example of a N protein of SARS-CoV-2 is given in NCBI protein database under the accession number QH062884.1.

The coronavirus S protein is a homotrimer of class I fusion glycoprotein that is divided into two functionally distinct parts (S1 and S2). The surface-exposed S1 contains the receptor-binding domain (RBD) that specifically engages the host cell receptor, thereby determining virus cell tropism and pathogenicity. The transmembrane S2 domain contains heptad repeat regions and the fusion peptide, which mediate the fusion of viral and cellular membranes upon extensive conformational rearrangements (Li, F. Structure, function, and evolution of coronavirus spike proteins. Annu. Rev. Virol. 3, 237-261 (2016), Letko, M., Marzi, A. & Munster, V. Functional assessment of cell entry and receptor usage for SARS-CoV-2 and other lineage B betacoronaviruses. Nat. Microbiol. 5, 562-569 (2020)). Example of a S protein of SARS-CoV-2 is given in NCBI protein database under the accession number QH062877.1.

Thus, in this embodiment, the antigen to which the single domain antibody, preferably the VHH (and in consequence the fusion protein comprising said single domain antibody) is directed may be S protein or N protein from a coronavirus. Preferably, the antigen is S protein or N protein of SARS-CoV-2, more preferably, the antigen is N protein of SARS-CoV-2 as people vaccinated against COVID-19 have been mostly immunised with the expression of the S protein.

In an another embodiment, the virus whose presence is to be diagnosed is the human immunodeficiency virus (HIV), advantageously HIV-1 and/or HIV-2. Recommendations are a combined ELISA of anti-HIV antibodies and P24 antigen. In this embodiment, the antigen to which the single domain antibody(ies), preferably VHH(s) (and in consequence the fusion protein(s) comprising said single domain antibody) is directed against P24, the HIV capsid component (277-363) proteolyzed from the protein GAG. The 282-351 amino acid sequence of P24 from HV1 (ELI/NDK isolate) used in the representative test is the following:

(SEQ ID NO: 155)
MADIRQGPKEPFRDYVDRFYKTLRAEQASQDVKNWMTETLLVQNANPDC
KTILKALGPQATLEEMMTACQ.

### A.2. Single Domain Antibodies

According to the invention, the first fusion protein comprises a first single domain antibody which is directed against a first epitope of the antigen and the second fusion protein comprises a second single domain antibody which is directed against a second epitope of the antigen.

The single domain antibody (from the fusion protein, the first fusion protein and/or the second fusion protein) is said to “specifically bind” its target antigen when the dissociation constant (K_{D}) is ≤10⁻⁷ M. The single domain antibody specifically binds antigen with “high affinity” when the K_{D} is ≤5×10⁻⁹ M, and with “very high affinity” when the K_{D} is ≤5×10⁻¹⁰ M.

In one embodiment, the single domain antibody binds the antigen with a K_{D}≤10⁻⁷ M, preferably between about 10⁻⁹ M and 10⁻¹³ M.

Single domain antibodies (sdAbs) encompass notably variable domain of camelid heavy-chain-only antibody (also called VHH) and variable domain of cartilaginous fish heavy-chain-only antibody (also called VNAR), variable domain of human heavy-chain antibody (sdhAb) or humanized proteins VHH (hVHH) or VNAR (hVNAR) by exchange of surface accessible residues out of CDR residues in VHH and VNAR structure by corresponding sequence-aligned residues from sdhAb. VHH may come from processed IgG gene from immunized camelids (vicuna, alpaca, llama, dromedary, camel), VNAR may come from processed IgG gene from immunized shrarks, sdhAb may come from processed IgG gene from immunized individuals or infected patients. VHH, VNAR, sdhAb or hVHH may be product by mutagenesis of their CDRs or from grafting CDR from each other or from full-size antibodies.

Thus, in some embodiments, the single domain antibody (sdAb) according to the invention is selected from the group consisting of variable domain of camelid heavy-chain antibody (VHH), cartilaginous fish heavy-chain antibody (VNAR), variable domain of human heavy-chain antibody (sdhAb), humanized VHH (hVHH) and humanized VNAR (hVNAR).

While binding to antigens with comparable affinity to that of conventional IgG, the following characteristics of single domain antibodies make them useful reagents for laboratory diagnosis:
low cost of production: the small size of single domain antibodies enables easier production and high yields in moderate volumes of bacterial culture,
easy tailoring to meet the application requirements (i.e., to improve specificity and affinity for broadening detection possibilities): the genes encoding single domain antibodies can be re-engineered to select for altered binding properties or epitope tagging for an immunoassay configuration,
robustness and long shelf live: single domain antibodies are exceptionally heat stable in comparison with Igs and ScFv fragments and can thus be easily shipped at most ambient temperatures,
targeting cryptic or hidden epitopes: the small size of a single domain antibody allows it to enter antigen-binding sites in protein pockets and cavities that might not be accessible to conventional antibodies (Tahir S Pillay, Serge Muyldermans, Application of Single-Domain Antibodies (“Nanobodies”) to Laboratory Diagnosis, Ann Lab Med. 2021 Nov. 1; 41(6):549-558).

In the context of the invention, the small size of single domains antibodies enables the first and second fusion protein to bind to the antigen while allowing the first and second fragments of luciferase to be close enough to restore the luciferase activity.

In the most preferred embodiment, the single domain antibody (first and second single domain antibody) is a variable domain of camelid heavy-chain-only antibody (VHH).

Camelids produce two kinds of immunoglobulin G antibodies (IgG): (i) conventional antibodies IgG made of dimers of heavy and light chains and (ii) a class of IgG devoid of light chain and made of dimers of heavy chains only (HC-IgGs) (Hamers-Casterman, C. et al. Naturally occurring antibodies devoid of light chains. Nature 363, 446-448 (1993)). The HC-IgGs comprise two antigen binding domains (referred to as VHH or nanobodies). VHHs are among the smallest available intact antigen binding fragments with a MW of only 15 kDa, 2.5 nm in diameter and ˜4 nm in height. They act as fully functional binding moieties and are easily produced in high amounts and in active form in E. coli. In addition, they exhibit unique characteristics, such as enlarged complementarity determining regions (CDRs) and the substitution of three to four hydrophobic framework residues (which interact with the VL in conventional antibodies) by more hydrophilic amino acids. To stabilize the enlarged CDRs, VHHs often possess an additional disulfide bond between CDR1 and CDR3 in dromedaries, and CDR2 and CDR3 in llamas (Harmsen, M. M. & De Haard, H. J. Properties, production, and applications of camelid single-domain antibody fragments. Appl. Microbiol. Biotechnol. 77, 13-22 (2007), Muyldermans, S. Single domain camel antibodies: current status. J. Biotechnol. 74, 277-302 (2001)). In particular the extended CDR3 loop can adopt a protruding conformation, which can interact with concave epitopes (Lauwereys, M. et al. Potent enzyme inhibitors derived from dromedary heavy-chain antibodies. EMBO J 17, 3512-3520 (1998)), whereas conventional antibodies recognize only convex or flat structures. These unique features allow VHHs to recognize novel epitopes that are poorly immunogenic for conventional antibodies (Lafaye, P., Achour, I., England, P., Duyckaerts, C. & Rougeon, F. Single-domain antibodies recognize selectively small oligomeric forms of amyloid β, prevent Aβ-induced neurotoxicity and inhibit fibril formation. Mol. Immunol. 46, (2009)). Over the last decades, VHHs have received progressively greater interest due to their specific properties. Indeed, they combine the high affinity and selectivity of conventional antibodies with the advantages of small molecules: in particular, they diffuse more readily into tissues owing to their small size and bind intracellular antigens and they are widely used for imaging (for a review, Traenkle, B. & Rothbauer, U. Under the Microscope: Single-Domain Antibodies for Live-Cell Imaging and Super-Resolution Microscopy. Front. Immunol. 8, 1030 (2017)). According to the invention, the first single domain antibody (sdAb) (and in consequence the first fusion protein) is directed against a first epitope of the antigen while the second sdAb (and in consequence the second fusion protein) is directed against a second epitope of the antigen. Preferably, the first and second epitopes must be chosen so that the first and the second sdAb s (and in consequence the first and the second fusion proteins) do not compete for their epitope. In a more general way, the first and the second epitopes are so that the binding of one of the fusion proteins to its epitope does not sterically hindered the other fusion protein to bind to its epitope. Therefore, preferably, the first and second epitopes are distinct. Thus, the first and the second VHHs may differ from at least one complementarity-determining region (CDR), preferably from at least two CDRs, most preferably from their three CDRs. The number and location of CDR region amino acid residues of herein comply with the known CDR numbering criteria such as Kabat (Kabat, EA, etc. 1991 Sequences of Proteins of Immunological Interest, 5th Ed), IMGT (IMGTO®:The Intemational ImMunoGeneTics Information System® http://www.imgt.org) or Chothia (Chothia C., Lesk A. M. Canonical structures for the hypervariable regions of immunoglobulins. Mol. Biol. 1987; 196:901-917. doi: 10.1016/0022-2836(87)90412-8.), preferably IMGT.

In a preferred embodiment, the first and the second single domain antibodies (and in consequence the first and the second fusion proteins) do not compete for their epitope and each of first and second single domain antibodies (and in consequence the first and the second fusion proteins) binds to the antigen with a K_{D}≤10⁻⁷ M, preferably between about 10⁻⁹ M and 10⁻¹³ M.

In the preferred embodiment where the first and second sdAbs are VHHs, the first VHH (and in consequence the first fusion protein) is directed against a first epitope of the antigen while the second VHH (and in consequence the second fusion protein) is directed against a second epitope of the antigen. Preferably, the first and second epitopes must be chosen so that the first and the second VHHs (and in consequence the first and the second fusion proteins) do not compete for their epitope. Preferably, the first and second epitopes are distinct. Thus, the first and the second VHHs may differ from at least one complementarity-determining region (CDR), preferably from at least two CDRs, most preferably from their three CDRs. The number and location of CDR region amino acid residues of herein comply with the known CDR numbering criteria such as Kabat, IMGT or Chothia, preferably IMGT.

In a particular embodiment, the first and the second epitopes may be identical but carried by different subunits assembled in the same entity as for example a homodimer as SARS-CoV-2 N protein, or a homotrimer as SARS-CoV-2 S protein. Thus, in an embodiment, the first and the second VHHs may be the same.

The term “compete” when used in the context of antigen binding proteins that compete for the same epitope means competition between antigen binding proteins as determined by an assay in which the antigen binding protein (e.g., antibody or in the context of the invention the sdAb, preferably the VHH, or the fusion protein comprising thereof) being tested prevents or inhibits (e.g., reduces) specific binding of a reference antigen binding protein (e.g., a ligand, or a reference antibody) to a common antigen (e.g., N protein or a fragment thereof). Numerous types of competitive binding assays can be used to determine if one antigen binding protein competes with another, using biophysical or biochemical approaches. Epitope location and overlap can be identified and mapped on antigen by biophysical approaches either by sdAb-antigen co-crystallization and structure resolution using X-ray diffraction, or lower differential hydrogen-deuterium exchange at sdAb-antigen interface measured by NMR or mass spectrometry. Several biochemical approaches are providing hints on sdAb binding site competition on antigens: historical methods are solid phase direct or indirect radioimmunoassay (RIA), solid phase direct or indirect enzyme immunoassay (EIA), sandwich competition assay (see, e.g., Stahli et al., 1983, Methods in Enzymology 9:242-253); solid phase direct biotin-avidin EIA (see, e.g., Kirkland et al., 1986, J. Immunol. 137:3614-3619) solid phase direct labelled assay, solid phase direct labelled sandwich assay (see, e.g., Harlow and Lane, 1988, Antibodies, A Laboratory Manual, Cold Spring Harbor Press); solid phase direct label RIA using 1-125 label (see, e.g., Morel et al., 1988, Molec. Immunol. 25:7-15); solid phase direct biotin-avidin EIA (see, e.g., Cheung, et al., 1990, Virology 176:546-552); and direct labelled RIA (Moldenhauer et al., 1990, Scand. J. Immunol. 32:77-82). More recent biochemical label free approaches use surface plasmon resonance (SPR) or bio-layer interferometry (BLI) for measuring the binding kinetic (kₒₙ and k_{off}) of sdAb to surface-bound antigens in flowing solution using optical measurements.

Typically, such an assay involves the use of purified antigen bound to a solid surface or cells bearing either of these, an unlabelled test antigen binding protein and a labelled reference antigen binding protein. Competitive inhibition is measured by determining the amount of label bound to the solid surface or cells in the presence of the test antigen binding protein. Usually the test antigen binding protein is present in excess. Antigen binding proteins identified by competition assay (competing antigen binding proteins) include antigen binding proteins binding to the same epitope as the reference antigen binding proteins and antigen binding proteins binding to an adjacent epitope sufficiently proximal to the epitope bound by the reference antigen binding protein for steric hindrance to occur. Additional details regarding methods for determining competitive binding are provided in the examples herein. Usually, when a competing antigen binding protein is present in excess, it will inhibit (e.g., reduce) specific binding of a reference antigen binding protein to a common antigen by at least 40-45%, 45-50%, 50-55%, 55-60%, 60-65%, 65-70%, 70-75% or 75% or more. In some instances, binding is inhibited by at least 80-85%, 85-90%, 90-95%, 95-97%, or 97% or more.

Thus, the methods disclose above may be used in order to test if the first sdAb, preferably the first VHH, and the second sdAb, preferably the second VHH, (or the first fusion protein and the second fusion protein) don't compete. For example, the first sdAb (or the first VHH or the first fusion protein) may be labelled and used as labelled reference antigen binding protein and the second sdAb (or the second VHH or the second fusion protein) may be used as test antigen binding protein (or conversely). When the test antigen binding protein (first or second sdAb, preferably VHH) which does not compete with reference antigen binding protein (labelled second or first sdAb, preferably VHH) is present in excess, it will inhibit the binding of the reference antigen binding protein (labelled second or first sdAb, preferably VHH) to the antigen which is to be detected by less 45-50%, 40-45%, 35-40%, 30-35%, 25-30% or 25% or less.

An example of epitope competition assay is given with a bioluminescence assay in multi-well plate. The first VHH (VHH1) is expressed as a fusion with a C-terminal 37 amino-acid long peptide (SBP37, SEQ ID NO: 62) presenting a high affinity for streptavidin (VHH1-SBP37: e.g., anti-N VHH655-SBP37, SEQ ID NO: 120 or anti-S VHH716-SBP37, SEQ ID NO: 118). This protein is loaded in a plate well coated with streptavidin. After a washing step, the antigen is added next and incubated. After a washing step the second VHH (VHH2) expressed as a C-terminal fusion with a fully active luciferase (SEQ ID NO: 4) is then added (VHH2-JAZ: e.g., anti-N VHH648-JAZ, SEQ ID NO: 119 or anti-S VHH687-JAZ, SEQ ID NO: 117). After a last washing step, the substrate is added and the light emission is measured (relative light intensity unit per second). If the light emission is in the background noise, either the epitope for the second fusion protein is not accessible on the antigen when the first fusion protein is bound or the second fusion protein affinity for the antigen is too low in measurement conditions. It is important to switch the VHH in the fusion proteins for testing the two combinations (VHH1-SBP37/VHH2-JAZ and VHH2-SBP37/VHH1-JAZ). This experiment may be also used by adding increasing amounts of free antigens with VHH-JAZ while loaded in the well with surface bound VHH-SBP37/antigen on coated streptavidin, the binding competition for VHH-JAZ between VHH-SBP37/antigen and free antigen allows a determination of the VHH-JAZ affinity (K_{D}) for the VHH-SBP37/antigen.

Preferably, when the antigen comprises several domains, the first and the second sdAbs, preferably VHHs are not directed against the same domain of said antigen (e.g. carboxy-terminal domain and amino-terminal domain of the N protein). The first and the second sdAbs, preferably VHHs, may also be directed to the same epitope but on a different monomer of a given multimer (e.g. they target multimers such as Nucleoprotein homodimers or Spike homotrimers on symmetrical or non-symmetrical epitopes).

As mentioned, above, the antigen to be detected may be a component from a pathogen selected from the group consisting of a virus, a bacteria, a fungus and a parasite or a fragment or part thereof. Thus, the detection of the antigen allows the detection of a pathogen and the diagnosis of an infectious pathology.

In another embodiment, the antigen to be detected may be a component expressed at the surface of a specific cell or in its cytoplasm or any of its organelles typically for diagnosing an inflammation or a cancer.

As mentioned above, in a most preferred embodiment, the sdAb (sdAb of the fusion protein, of the first fusion protein and/or of the second fusion protein) is a VHH.

The VHH may be selected among known VHHs. It is known VHHs raised to numerous pathogens (reviewed in Vanlandschoot, P. et al. Nanobodies®: new ammunition to battle viruses. Antiviral Res. 92, 389-407 (2011) and Lafaye, P. & Li, T. Use of camel single-domain antibodies for the diagnosis and treatment of zoonotic diseases. Comp Immunol Microbiol Infect Dis 60, 17-22 (2018)) including:
HIV (Forsman, A. et al. Llama antibody fragments with cross-subtype human immunodeficiency virus type 1 (HIV-1)-neutralizing properties and high affinity for HIV-1 gp120. J. Virol. 82, 12069-12081 (2008), McCoy, L. E. et al. Potent and broad neutralization of HIV-1 by a llama antibody elicited by immunization. J. Exp. Med. (2012));
Influenza A (Hultberg, A. et al. Llama-derived single domain antibodies to build multivalent, superpotent and broadened neutralizing anti-viral molecules. PLoS One 6, 1-12 (2011), Ashour, J. et al. Intracellular expression of camelid single-domain antibodies specific for influenza virus nucleoprotein uncovers distinct features of its nuclear localization. J. Virol. 89, 2792-800 (2015), Laursen, N. et al. Universal protection against influenza infection by a multidomain antibody to influenza hemagglutinin. Science. 362, 598-602 (2018));
Poliovirus (Thys, B. et al. In vitro antiviral activity of single domain antibody fragments against poliovirus. Antiviral Res. (2010));
rabies virus;
Foot and Mouth Disease Virus (Harmsen, M. M. & De Haard, H. J. Properties, production, and applications of camelid single-domain antibody fragments. Appl. Microbiol. Biotechnol. 77, 13-22 (2007));
rotavirus (van der Vaart, J. M. et al. Reduction in morbidity of rotavirus induced diarrhoea in mice by yeast produced monovalent llama-derived antibody fragments. Vaccine 24, 4130-4137 (2006)), HCV (Tarr, A. W. et al. An alpaca nanobody inhibits hepatitis C virus entry and cell-to-cell transmission. Hepatology 58, 932-939 (2013)),
and recently SARS, MERS and SARS-Cov-2 spike proteins (Wrapp, D. et al. Structural Basis for Potent Neutralization of Betacoronaviruses by Single-Domain Camelid Antibodies. Cell 181, 1004-1015.e15 (2020), Huo, J. et al. Neutralizing nanobodies bind SARS-CoV-2 spike RBD and block interaction with ACE2. Nat. Struct. Mol. Biol. (2020)).

VHH to be used according to the invention may be also selected from a library. Methods, such as phage (e.g. M13, fusion with PIII), bacterium (e.g. E. coli, fusion with intimin), yeast (e.g. S. cerevisae, fusion with AgaP2) or ribosome display, have been described to select antigen-specific VHH either from VHH libraries of either immunized camelids or from synthetic library using naive VHH scaffolds with synthetic oligonucleotide-encoded CDRs. For example, the VHH genes from immunized camelids such as immunized alpaca are cloned in phage display vectors (e.g. M13, VHH fusion with PIII), the antigen binders are obtained by panning and selected VHH are expressed in bacteria (e.g. E. coli). The recombinant VHHs have a number of advantages compared with the conventional antibody fragments (Fab or scFv), because only one domain has to be cloned and because these VHHs are well expressed, highly soluble in aqueous environments and are stable at high temperature.

VHH may also be custom designed, screened from synthetic libraries derivatized from camelid VHH scaffold or from humanized scFv scaffold.

For example, the VHH is obtainable by the method comprising the steps of:
(a) immunizing a camelid, preferably a Lama pacos (alpaca), with the immunoglobulin or a fragment thereof,
(b) isolating peripheral lymphocytes of the immunized camelid, obtaining the total RNA and synthesizing the corresponding cDNAs (methods are known in the art; for instance, see Lafaye et al. 1995 Res Immunol., 146, 373-82; Erratum in: 1996, Res Immunol., 147, 61),
(c) constructing a library of cDNA fragments encoding VHH domains,
(d) selecting the VHH domain in the library.

The selection of the VHH domain in the library may be carried out by the following method:
(d1) subcloning the cDNA fragments as fusion with the PIII gene of M13 in the phage display vector (pHEN6),
(d2) transforming TG1 F′ strain of E. coli,
(d3) adding the helper phage M13 KO7 to the pooled transformants for producing recombinant phages expressing the VHH at their surface in the culture media,
(d4) concentrating recombinant phages in polyethylene glycol (MW 4000 Da) and titrating,
(d5) immobilizing antigens either on recipient wall (tube or plate well) or magnetic beads then antigen-specific VHH are selected by phage display in 2-3 rounds
(d6) sequencing VHH genes from phagemids, counting occurrences of identical CDR,
(d7) subcloning selected and non-redundant VHH, expressing in E. coli, purifying and measuring their affinity for the antigen.

In the embodiment wherein the antigen is N protein, preferably the SARS-CoV-2 N protein, the first and second sdAbs, preferably VHHs, both bind to N protein, preferably the SARS-CoV-2 N protein.

In some embodiments the first and second sdAbs, preferably VHHs, both bind a protein comprising the amino acid sequence of the SARS-CoV-2 N protein of NCBI QH062884.1.

In some embodiments the first and/or second sdAb, preferably VHH, binds to the C-terminal domain (CTD) of N protein, preferably the N protein of SARS-CoV-2.

In some embodiments the first and/or second sdAb, preferably VHH bind to the N-terminal domain (NTD) of N protein, preferably the N protein of SARS-CoV-2.

Preferably, if the first sdAb binds to the C-terminal domain of N protein, the second sdAb binds to the N-terminal domain of N protein. In the same way, if the first sdAb binds to the N-terminal domain of N protein, the second binds to the C-terminal domain of N protein. Having a first and a second sdAbs binding two different domains from N protein enables the first and second fusion proteins comprising them not to compete for their epitopes nor to sterically hinder each other.

In the embodiment where first and second sdAbs are VHHs, preferably, if the first VHH binds to the C-terminal domain of N protein, the second VHH binds to the N-terminal domain of N protein. In the same way, if the first VHH binds to the N-terminal domain of N protein, the second binds to the C-terminal domain of N protein. Having a first and a second VHHs binding two different domains from N protein enables the first and second fusion proteins comprising them not to compete for their epitopes nor to sterically hinder each other.

Given that SARS-CoV-2 N is a homodimer, each one of the two fusion proteins may bind each one of the two monomers on symmetrical or non-symmetrical epitopes.

The same reasoning applies for S protein. In the embodiment wherein the antigen is S protein, the first and second VHH both binds to S protein. In some embodiments the first and second sdAbs, preferably VHHs, both bind a protein comprising the amino acid sequence of the SARS-CoV-2 S protein of NCBI QH062877.1.

The first and/or second sdAb may bind to S1 part of S protein. In another embodiment, the first and/or second sdAb binds to the S2 part of S protein. If the first sdAb binds to the S1 part of S protein, the second sdAb binds preferably to the S2 part of S protein. Reciprocally, if the first sdAb binds to the S2 part of S protein, the second sdAb binds to the S1 part of S protein.

The first and/or second VHH may bind to S1 part of S protein. In another embodiment, the first and/or second VHH binds to the S2 part of S protein. If the first VHH binds to the S1 part of S protein, the second VHH binds preferably to the S2 part of S protein. Reciprocally, if the first VHH binds to the S2 part of S protein, the second VHH binds to the S1 part of S protein.

Given that SARS-CoV-2 S is a homotrimer, each one of the two fusion proteins may also bind each one of the monomers on symmetrical or non-symmetrical epitopes.

The N protein and/or the S protein are preferably the N protein and/or the S protein of SARS-CoV-2.

Embodiment where the Antigen is the N Protein of SARS-CoV-2

Examples of VHHs binding to N protein of SARS-CoV-2 are given in Table 1 below.

TABLE 1

| | VHH sequence | |
|---|---|---|
| VHH | identification | |
| Name | number | Amino acid sequence |
| D12-1 | SEQ ID NO: 20 | EVQLVESGGGLVQPGGSLRLSCTVSEFSLRWNAIGWFRQAPGKEREGVSCISSNGAYTYIADSVKGRFAI |
| | | STDSVKKMVYLQMNMLKPEDTAVYYCATGSPGCYSAVDEFPYWGRGTQVTVSS |
| H3-3 | SEQ ID NO: 21 | EVQLVESGGGLVQAGGSLRLSCAASGRTFSSYAMGWFRQAPGKEREFVAAIGWMVGSIYYADSVKDRFT |
| | | ISRDNAKNTVYLQMNSLKPEDTAVYYCAAELGGSYLSWRDYGMDYWGKGTLVTVSS |
| E7-2 | SEQ ID NO: 22 | EVQLVESGGGLVQAGDSLRLSCAASGRTFSNYAMGWFRQAPGKEEREFVAAISRDGGFKFYAESVKGRF |
| | | TISRDIARDTVYLQMNSLKPEDTAVYYCAAKSNTYFSDGIITSRTQYDYWGQGTQVTVSS |
| G9-1 | SEQ ID NO: 23 | EVQLVESGGGLVePGGSLRLSCAASGFTWDYYDIGWFRQAPGKEREGVACISSSGSSTNYGDSVKGRFTI |
| | | SRDNAKKTVYLQMNSLKPEDTAVYYCAADIVDYGLESASCMWIDRGYWGQGTQVTVSS |
| E10-3 | SEQ ID NO: 24 | EVQLVESGGGLVQPGGSLRLSCAASGFGLDYYAIGWFRQAPGKEREGVSCISNSGRSTNPADSVKGRFTI |
| | | SRDNAKNTVYLQMNSLKPEDTAVYYCAATAWRHACTHISNEYDYWGQGTQVTVSS |
| C7-1 | SEQ ID NO: 25 | EVQLQASGGGLVQPGGSLRLSCAASGFTLGYYRIGWFRQAPGKEREGVSCLSSSGRSTNYADSVKGRFT |
| | | ISTDNAKNTVYLQMDSLKPEDTAVYYCAADFTPGPRLCSILSLNEYSAWGQGTQVTVSS |
| F11-1 | SEQ ID NO: 26 | EVQLVESGGGLVQPGGSLRLSCAASGFTSDYYVIGWFRQAPGKEREGVSCISSGGGSTNYADSVKGRFTI |
| | | SRDNAKNTVYLQMNSLKPEDTAVYYCAALNRIHYYSCSVLMGDYGSWGQGTQVTVSS |
| E4-3 | SEQ ID NO: 27 | EVQLVESGGGLVQPGGSLRLSCAASGFTLDYYAIYWFRQAPGKEREGVSCISSSGGSTNYADSVKGRFTI |
| | | SRDNAKNTVYLQMNSLKPEDTAVYYCAAGPSECGYSDYLDYWGQGTQVTVSS |
| H7-1 | SEQ ID NO: 28 | EVQLQASGGGLVQAGGSLRLSCAASGRTFSSYAMGWFREAPGKEREFVAAISWSGAGTYYADSVKGRF |
| | | TISRDNAKNTVYLQMNSLKPEDTAVYYCAAPSAVVAGTYVADYDYWGQGTQVTVSS |
| B6-1 | SEQ ID NO: 29 | QVQLVESGGGLVQAGGSLRLSCAASGRSFSNYNTAWFRQAPGKEREFVALISWTVGNTPYADSVKGRFT |
| | | ISRDNAKNTVYLQMNSLNAEDTAVYYCAAGRPSIYYRTYDRYDYWGQGTQVTVSS |

The CDRs of the VHHs anti N protein of Table 1 are given in the Table 2 below.

TABLE 2
identification number

| | | CDR sequence | |
|---|---|---|---|
| | | identification | |
| VHH Name | CDR | number | Amino acid sequence |
| D12-1 | CDR1 | SEQ ID NO: 30 | SEFSLRWNAIG |
| | CDR2 | SEQ ID NO: 31 | SCISSNGAYTYIADSVKG |
| | CDR3 | SEQ ID NO: 32 | ATGSPGCYSAVDEFPY |
| H3-3 | CDR1 | SEQ ID NO: 33 | SGRTFSSYAMG |
| | CDR2 | SEQ ID NO: 34 | AAIGWMVGSIYYADSVKD |
| | CDR3 | SEQ ID NO: 35 | AAELGGSYLSWRDYGMDY |
| E7-2 | CDR1 | SEQ ID NO: 36 | SGRTFSNYAMG |
| | CDR2 | SEQ ID NO: 37 | AAISRDGGFKFYAESVKG |
| | CDR3 | SEQ ID NO: 38 | AAKSNTYFSDGIITSRTQYDY |
| G9-1 | CDR1 | SEQ ID NO: 39 | SGFTWDYYDIG |
| | CDR2 | SEQ ID NO: 40 | ACISSSGSSTNYGDSVKG |
| | CDR3 | SEQ ID NO: 41 | AADIVDYGLESASCMWIDRGY |
| E10-3 | CDR1 | SEQ ID NO: 42 | SGFGLDYYAIG |
| | CDR2 | SEQ ID NO: 43 | SCISNSGRSTNPADSVKG |
| | CDR3 | SEQ ID NO: 44 | AATAWRHACTHISNEYDY |
| C7-1 | CDR1 | SEQ ID NO: 45 | SGFTLGYYRIG |
| | CDR2 | SEQ ID NO: 46 | SCLSSSGRSTNYADSVKG |
| | CDR3 | SEQ ID NO: 47 | AADFTPGPRLCSILSLNEYSA |
| F11-1 | CDR1 | SEQ ID NO: 48 | SGFTSDYYVIG |
| | CDR2 | SEQ ID NO: 49 | SCISSGGGSTNYADSVKG |
| | CDR3 | SEQ ID NO: 50 | AALNRIHYYSCSVLMGDYGS |
| E4-3 | CDR1 | SEQ ID NO: 51 | SGFTLDYYAIY |
| | CDR2 | SEQ ID NO: 52 | SCISSSGGSTNYADSVKG |
| | CDR3 | SEQ ID NO: 53 | AAGPSECGYSDYLDY |
| H7-1 | CDR1 | SEQ ID NO: 54 | SGRTFSSYAMG |
| | CDR2 | SEQ ID NO: 55 | AAISWSGAGTYYADSVKG |
| | CDR3 | SEQ ID NO: 56 | AAPSAVVAGTYVADYDY |
| B6-1 | CDR1 | SEQ ID NO: 57 | SGRSFSNYNTA |
| | CDR2 | SEQ ID NO: 58 | ALISWTVGNTPYADSVKG |
| | CDR3 | SEQ ID NO: 59 | AAGRPSIYYRTYDRYDY |

A XL1 blue E. coli transformed with a pASK vector wherein the gene coding for the VHH C7-1 is cloned and which expresses the VHH C7-1 (also named VHH N-NTD C7-1) in the periplasm after induction with anhydrotétracycline (AHT) (0.2 μg/ml overnight at 16° C.) was deposited, according to the Budapest Treaty, at CNCM (Collection Nationale de Cultures de Microorganismes, 25 rue du Docteur Roux, 75724 Paris Cedex 15, France) on Oct. 7, 2020 with the number I-5601.

A XL1 blue E. coli transformed with a pASK vector wherein the gene coding for the VHH G9-1 is cloned and which expresses the VHH G9-1 (also named VHH N-CTD G9-1) in the periplasm after induction with anhydrotétracycline (AHT) (0.2 μg/ml overnight at 16° C.) was deposited, according to the Budapest Treaty, at CNCM (Collection Nationale de Cultures de Microorganismes, 25 rue du Docteur Roux, 75724 Paris Cedex 15, France) on Oct. 7, 2020 with the number I-5603.

VHHs E7-2, G9-1, H3-3, D12-1, E10-3 recognize the CTD of N protein. The VHHs B6-1, C7-1, F11-1, H7-1 and E4-3 recognize the NTD of N protein.

Thus, the first and/or the second sdAb may comprise:

### CDR1:

having the amino acid sequence selected from the group consisting of SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54 and SEQ ID NO: 57 and
variant thereof having up to two amino acids additions deletions and/or substitutions compared to amino acids sequences selected from the group consisting of SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54 and SEQ ID NO: 57,

### CDR2:

having the amino acid sequence selected from the group consisting of SEQ ID NO: 31, SEQ ID NO: 34, SEQ ID NO: 37, SEQ ID NO: 40, SEQ ID NO: 43, SEQ ID NO: 46, SEQ ID NO: 49, SEQ ID NO: 52, SEQ ID NO: 55 and SEQ ID NO: 58 and
variant thereof having up to two amino acids additions deletions and/or substitutions compared to amino acids sequences selected from the group consisting of SEQ ID NO: 31, SEQ ID NO: 34, SEQ ID NO: 37, SEQ ID NO: 40, SEQ ID NO: 43, SEQ ID NO: 46, SEQ ID NO: 49, SEQ ID NO: 52, SEQ ID NO: 55 and SEQ ID NO: 58,

### CDR3:

having the amino acid sequence selected from the group consisting of SEQ ID NO: 32, SEQ ID NO: 35, SEQ ID NO: 38, SEQ ID NO: 41, SEQ ID NO: 44, SEQ ID NO: 47, SEQ ID NO: 50, SEQ ID NO: 53, SEQ ID NO: 56 and SEQ ID NO: 59 and
variant thereof having up to two amino acids additions deletions and/or substitutions compared to amino acids sequences selected from the group consisting of SEQ ID NO: 32, SEQ ID NO: 35, SEQ ID NO: 38, SEQ ID NO: 41, SEQ ID NO: 44, SEQ ID NO: 47, SEQ ID NO: 50, SEQ ID NO: 53, SEQ ID NO: 56 and SEQ ID NO: 59.

The first and/or second sdAbs are preferably VHHs. Thus, the first and/or the second VHH may comprise:

### CDR1:

having the amino acid sequence selected from the group consisting of SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54 and SEQ ID NO: 57 and
variant thereof having up to two amino acids additions deletions and/or substitutions compared to amino acids sequences selected from the group consisting of SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54 and SEQ ID NO: 57,

### CDR2:

having the amino acid sequence selected from the group consisting of SEQ ID NO: 31, SEQ ID NO: 34, SEQ ID NO: 37, SEQ ID NO: 40, SEQ ID NO: 43, SEQ ID NO: 46, SEQ ID NO: 49, SEQ ID NO: 52, SEQ ID NO: 55 and SEQ ID NO: 58 and
variant thereof having up to two amino acids additions deletions and/or substitutions compared to amino acids sequences selected from the group consisting of SEQ ID NO: 31, SEQ ID NO: 34, SEQ ID NO: 37, SEQ ID NO: 40, SEQ ID NO: 43, SEQ ID NO: 46, SEQ ID NO: 49, SEQ ID NO: 52, SEQ ID NO: 55 and SEQ ID NO: 58,

### CDR3:

having the amino acid sequence selected from the group consisting of SEQ ID NO: 32, SEQ ID NO: 35, SEQ ID NO: 38, SEQ ID NO: 41, SEQ ID NO: 44, SEQ ID NO: 47, SEQ ID NO: 50, SEQ ID NO: 53, SEQ ID NO: 56 and SEQ ID NO: 59 and
variant thereof having up to two amino acids additions deletions and/or substitutions compared to amino acids sequences selected from the group consisting of SEQ ID NO: 32, SEQ ID NO: 35, SEQ ID NO: 38, SEQ ID NO: 41, SEQ ID NO: 44, SEQ ID NO: 47, SEQ ID NO: 50, SEQ ID NO: 53, SEQ ID NO: 56 and SEQ ID NO: 59.

In an embodiment, the first and the second sdAbs, the first and the second sdAbs being preferably VHHs, are not directed against the same epitope. Therefore, advantageously, in this embodiment, the first and the second sdAbs, being preferably VHHs, differ. The first and the second sdAbs being preferably VHHs, may differ from at least one complementarity-determining region (CDR), preferably from at least two CDRs, most preferably from their three CDRs.

In an embodiment, the first and/or the second sdAbs comprise:

CDR1, CDR2 and CDR3 having respectively the amino acid sequences selected from the groups:
SEQ ID NO: 30, SEQ ID NO: 31 and SEQ ID NO: 32,
SEQ ID NO: 33, SEQ ID NO: 34 and SEQ ID NO: 35,
SEQ ID NO: 36, SEQ ID NO: 37 and SEQ ID NO: 38,
SEQ ID NO: 39, SEQ ID NO: 40 and SEQ ID NO: 41,
SEQ ID NO: 42, SEQ ID NO: 43 and SEQ ID NO: 44,
SEQ ID NO: 45, SEQ ID NO: 46 and SEQ ID NO: 47,
SEQ ID NO: 48, SEQ ID NO: 49 and SEQ ID NO: 50,
SEQ ID NO: 51, SEQ ID NO: 52 and SEQ ID NO: 53,
SEQ ID NO: 54, SEQ ID NO: 55 and SEQ ID NO: 56 and
SEQ ID NO: 57, SEQ ID NO: 58 and SEQ ID NO: 59.

In an embodiment, the first and/or the second VHHs comprise:

CDR1, CDR2 and CDR3 having respectively the amino acid sequences selected from the groups:
SEQ ID NO: 30, SEQ ID NO: 31 and SEQ ID NO: 32,
SEQ ID NO: 33, SEQ ID NO: 34 and SEQ ID NO: 35,
SEQ ID NO: 36, SEQ ID NO: 37 and SEQ ID NO: 38,
SEQ ID NO: 39, SEQ ID NO: 40 and SEQ ID NO: 41,
SEQ ID NO: 42, SEQ ID NO: 43 and SEQ ID NO: 44,
SEQ ID NO: 45, SEQ ID NO: 46 and SEQ ID NO: 47,
SEQ ID NO: 48, SEQ ID NO: 49 and SEQ ID NO: 50,
SEQ ID NO: 51, SEQ ID NO: 52 and SEQ ID NO: 53,
SEQ ID NO: 54, SEQ ID NO: 55 and SEQ ID NO: 56 and
SEQ ID NO: 57, SEQ ID NO: 58 and SEQ ID NO: 59.

In an embodiment, the first and/or the second sdAb comprises CDR1, CDR2 and CDR3 having respectively the amino acid sequences SEQ ID NO: 39, SEQ ID NO: 40 and SEQ ID NO: 41.

In an embodiment, the first and/or the second VHHs comprises CDR1, CDR2 and CDR3 having respectively the amino acid sequences SEQ ID NO: 39, SEQ ID NO: 40 and SEQ ID NO: 41.

In an embodiment, the first and/or the second sdAb comprises CDR1, CDR2 and CDR3 having respectively the amino acid sequences SEQ ID NO: 45, SEQ ID NO: 46 and SEQ ID NO: 47.

In an embodiment, the first and/or the second VHHs comprises CDR1, CDR2 and CDR3 having respectively the amino acid sequences SEQ ID NO: 45, SEQ ID NO: 46 and SEQ ID NO: 47.

In an embodiment, the first sdAb comprises CDR1, CDR2 and CDR3 having respectively the amino acid sequences SEQ ID NO: 39, SEQ ID NO: 40 and SEQ ID NO: 41 and the second sdAb comprises CDR1, CDR2 and CDR3 having respectively the amino acid sequences SEQ ID NO: 45, SEQ ID NO: 46 and SEQ ID NO: 47.

In an embodiment, the first VHH comprises CDR1, CDR2 and CDR3 having respectively the amino acid sequences SEQ ID NO: 39, SEQ ID NO: 40 and SEQ ID NO: 41 and the second VHH comprises CDR1, CDR2 and CDR3 having respectively the amino acid sequences SEQ ID NO: 45, SEQ ID NO: 46 and SEQ ID NO: 47.

In another embodiment, the first sdAb comprises CDR1, CDR2 and CDR3 having respectively the amino acid sequences SEQ ID NO: 45, SEQ ID NO: 46 and SEQ ID NO: 47 and the second sdAb comprises CDR1, CDR2 and CDR3 having respectively the amino acid sequences SEQ ID NO: 39, SEQ ID NO: 40 and SEQ ID NO: 41.

In another embodiment, the first VHH comprises CDR1, CDR2 and CDR3 having respectively the amino acid sequences SEQ ID NO: 45, SEQ ID NO: 46 and SEQ ID NO: 47 and the second VHH comprises CDR1, CDR2 and CDR3 having respectively the amino acid sequences SEQ ID NO: 39, SEQ ID NO: 40 and SEQ ID NO: 41.

Advantageously, the first and/or second VHH comprises:
an amino acid sequence selected from the group consisting of SEQ ID NO: 20 to 29 or
an amino acid sequence that is at least 80%, at least 85%, at least 90%, at least 95%, at least 97% or at least 99% identical to an amino acid sequence selected from the group consisting of SEQ ID NO: 20 to 29.

In an embodiment, the first and/or second VHH consists of:
an amino acid sequence selected from the group consisting of SEQ ID NO: 20 to 29 or
an amino acid sequence that is at least 80%, at least 85%, at least 90%, at least 95%, at least 97% or at least 99% identical to an amino acid sequence selected from the group consisting of SEQ ID NO: 20 to 29.

In a preferred embodiment, the first and/or second VHH comprises an amino acid sequence selected from the group consisting of SEQ ID NO: 20 to 29.

In a preferred embodiment, the first and/or second VHH consist of an amino acid sequence selected from the group consisting of SEQ ID NO: 20 to 29.

In a more preferred embodiment, one of the first VHH or the second VHH is directed against the CTD of the N protein whereas the other VHH is directed against the NTD of the N protein.

In a more preferred embodiment, the first or second VHH comprises:
an amino acid sequence selected from the group consisting of SEQ ID NO: 20 to 24 or
an amino acid sequence that is at least 80%, at least 85%, at least 90%, at least 95%, at least 97% or at least 99% identical to an amino acid sequence selected from the group consisting of SEQ ID NO: 20 to 24.

In a more preferred embodiment, the first or second VHH consists of:
an amino acid sequence selected from the group consisting of SEQ ID NO: 20 to 24 or
an amino acid sequence that is at least 80%, at least 85%, at least 90%, at least 95%, at least 97% or at least 99% identical to an amino acid sequence selected from the group consisting of SEQ ID NO: 20 to 24.

In a more preferred embodiment, the first or second VHH comprises an amino acid sequence selected from the group consisting of SEQ ID NO: 20 to 24.

In a more preferred embodiment, the first or second VHH consists of an amino acid sequence selected from the group consisting of SEQ ID NO: 20 to 24.

In a more preferred embodiment, the first or second VHH comprises:
an amino acid sequence selected from the group consisting of SEQ ID NO: 25 to 29 or
an amino acid sequence that is at least 80%, at least 85%, at least 90%, at least 95%, at least 97% or at least 99% identical to an amino acid sequence selected from the group consisting of SEQ ID NO: 25 to 29.

In a more preferred embodiment, the first or second VHH consists of:
an amino acid sequence selected from the group consisting of SEQ ID NO: 25 to 29 or
an amino acid sequence that is at least 80%, at least 85%, at least 90%, at least 95%, at least 97% or at least 99% identical to an amino acid sequence selected from the group consisting of SEQ ID NO: 25 to 29.

In a more preferred embodiment, the first or second VHH comprises an amino acid sequence selected from the group consisting of SEQ ID NO: 25 to 29.

In a more preferred embodiment, the first or second VHH consists of an amino acid sequence selected from the group consisting of SEQ ID NO: 25 to 29.

In an embodiment, the first VHH comprises:
an amino acid sequence selected from the group consisting of SEQ ID NO: 20 to 24 or
an amino acid sequence that is at least 80%, at least 85%, at least 90%, at least 95%, at least 97% or at least 99% identical to an amino acid sequence selected from the group consisting of SEQ ID NO: 20 to 24
   and
   the second VHH comprises:
an amino acid sequence selected from the group consisting of SEQ ID NO: 25 to 29 or
an amino acid sequence that is at least 80%, at least 85%, at least 90%, at least 95%, at least 97% or at least 99% identical to an amino acid sequence selected from the group consisting of SEQ ID NO: 25 to 29.

In an embodiment, the first VHH consists of:
an amino acid sequence selected from the group consisting of SEQ ID NO: 20 to 24 or
an amino acid sequence that is at least 80%, at least 85%, at least 90%, at least 95%, at least 97% or at least 99% identical to an amino acid sequence selected from the group consisting of SEQ ID NO: 20 to 24
   and
   the second VHH consists of:
an amino acid sequence selected from the group consisting of SEQ ID NO: 25 to 29 or
an amino acid sequence that is at least 80%, at least 85%, at least 90%, at least 95%, at least 97% or at least 99% identical to an amino acid sequence selected from the group consisting of SEQ ID NO: 25 to 29.

In an embodiment, the first VHH comprises an amino acid sequence selected from the group consisting of SEQ ID NO: 20 to 24 and the second VHH comprises an amino acid sequence selected from the group consisting of SEQ ID NO: 25 to 29.

In an embodiment, the first VHH consists of an amino acid sequence selected from the group consisting of SEQ ID NO: 20 to 24 and the second VHH consists of an amino acid sequence selected from the group consisting of SEQ ID NO: 25 to 29.

In an embodiment, the first VHH comprises:
an amino acid sequence selected from the group consisting of SEQ ID NO: 25 to 29 or
an amino acid sequence that is at least 80%, at least 85%, at least 90%, at least 95%, at least 97% or at least 99% identical to an amino acid sequence selected from the group consisting of SEQ ID NO: 25 to 29
   and
   the second VHH comprises:
an amino acid sequence selected from the group consisting of SEQ ID NO: 20 to 24 or
an amino acid sequence that is at least 80%, at least 85%, at least 90%, at least 95%, at least 97% or at least 99% identical to an amino acid sequence selected from the group consisting of SEQ ID NO: 20 to 24.

In an embodiment, the first VHH consists of:
an amino acid sequence selected from the group consisting of SEQ ID NO: 25 to 29 or
an amino acid sequence that is at least 80%, at least 85%, at least 90%, at least 95%, at least 97% or at least 99% identical to an amino acid sequence selected from the group consisting of SEQ ID NO: 25 to 29
   and
   the second VHH consists of:
an amino acid sequence SEQ ID NO: 20 to 24 or
an amino acid sequence that is at least 80%, at least 85%, at least 90%, at least 95%, at least 97% or at least 99% identical to an amino acid sequence selected from the group consisting of SEQ ID NO: 20 to 24.

In an embodiment, the first VHH comprises an amino acid sequence selected from the group consisting of SEQ ID NO: 25 to 29 and the second VHH comprises an amino acid sequence SEQ ID NO: 20 to 24.

In an embodiment, the first VHH consists of an amino acid sequence selected from the group consisting of SEQ ID NO: 25 to 29 and the second VHH consists of an amino acid sequence selected from the group consisting of SEQ ID NO: 20 to 24.

In an embodiment, the first VHH comprises:
an amino acid sequence SEQ ID NO: 23 or
an amino acid sequence that is at least 80%, at least 85%, at least 90%, at least 95%, at least 97% or at least 99% identical to an amino acid sequence SEQ ID NO: 23,
   and
   the second VHH comprises:
an amino acid sequence SEQ ID NO: 25 or
an amino acid sequence that is at least 80%, at least 85%, at least 90%, at least 95%, at least 97% or at least 99% identical to an amino acid sequence SEQ ID NO: 25.

In an embodiment, the first VHH consists of:
an amino acid sequence SEQ ID NO: 23 or
an amino acid sequence that is at least 80%, at least 85%, at least 90%, at least 95%, at least 97% or at least 99% identical to an amino acid sequence SEQ ID NO: 23,
   and
   the second VHH consists of:
an amino acid sequence SEQ ID NO: 25 or
an amino acid sequence that is at least 80%, at least 85%, at least 90%, at least 95%, at least 97% or at least 99% identical to an amino acid sequence SEQ ID NO: 25.

In an alternative embodiment, the first VHH comprises:
an amino acid sequence SEQ ID NO: 25 or
an amino acid sequence that is at least 80%, at least 85%, at least 90%, at least 95%, at least 97% or at least 99% identical to an amino acid sequence SEQ ID NO: 25
   and
   the second VHH comprises:
an amino acid sequence SEQ ID NO: 23 or
an amino acid sequence that is at least 80%, at least 85%, at least 90%, at least 95%, at least 97% or at least 99% identical to an amino acid sequence SEQ ID NO: 23.

In an alternative embodiment, the first VHH consists of:
an amino acid sequence SEQ ID NO: 25 or
an amino acid sequence that is at least 80%, at least 85%, at least 90%, at least 95%, at least 97% or at least 99% identical to an amino acid sequence SEQ ID NO: 25
   and
   the second VHH consists of:
an amino acid sequence SEQ ID NO: 23 or
an amino acid sequence that is at least 80%, at least 85%, at least 90%, at least 95%, at least 97% or at least 99% identical to an amino acid sequence SEQ ID NO: 23.

In a most preferred embodiment, the first VHH comprises the amino acid sequence SEQ ID NO: 23 and the second VHH comprises the amino acid sequence SEQ ID NO: 25.

In a most preferred embodiment, the first VHH consists of the amino acid sequence SEQ ID NO: 23 and the second VHH consists of the amino acid sequence SEQ ID NO: 25.

In an alternative embodiment, the first VHH comprises the amino acid sequence SEQ ID NO: 25 and the second VHH comprises the amino acid sequence SEQ ID NO: 23.

In an alternative embodiment, the first VHH consists of the amino acid sequence SEQ ID NO: 25 and the second VHH consists of the amino acid sequence SEQ ID NO: 23.

Embodiment where the Antigen is the S Protein of SARS-CoV-2.

Examples of VHHs binding to S protein of SARS-CoV-2 are given in Table 3 below.

TABLE 3

| | VHH sequence | |
|---|---|---|
| VHH Name | identification | |
| (Target) | number | Amino acid sequence |
| P_S12 | SEQ ID NO: 78 | MAEVQLQASGGGLVEAGGSLRLSCTTSGLTFSSVTMGWFRQAPGKEREFVAAIRWKFGNLGYADSVKG |
| (S-RBD) | | RFTVSRDNAKNTVYLQMNSLKPEDTAVYYCAAARVGEIIAVLISPSNYAYWGQGTQVTVSS |
| P_H08 | SEQ ID NO: 79 | MAEVQLQASGGGLVQPGGSLRLSCAASGSFFSISAMGWYRQAPGKQRELVADITSGGSTNYADSVKGR |
| (S-NTD) | | FTISRDNAKNTVYLQMNSLKPEDTAVYYCHVQVGVHPIGYDVWGQGTQVTVS |
| P_S11 | SEQ ID NO: | MAQVQLVESGGGLVQAGDSLRLSCAVSGRTFSSLIMGWFRQAPGKEREFVARITYSGGSTHYADSVKG |
| (S-NTD) | 80 | RFTISRDNAKNTVYLQMNSLKPEDTAVYYCAADTRGFSWSSSGGYDYWGQGTQVTVASEPKTPKPQP |
| P_F04-3 | SEQ ID NO: | MAEVQLVESGGGLVQAGGSLRLSCAASGRAFSRYFMGWFRQAPGKEREFVAGISRSGGSTDYANFVK |
| (S-RBD) | 126 | GRFTISRDNAKNTVYLQMNSLKPEDTAVYYCAATVDYSGTLTAARGREDYDDWGQGIQVTVSS |
| P_G09-1 | SEQ ID NO: | MAEVQLVESGGGTVQPGGSLRLSCEVSGTGFTINAMGWDRQAPGKQRELVATITRGDRIH |
| (S-RBD) | 127 | YADSVKGRFAISRDKDKNTVYLEMNNLKPEDTAVYYCDVAAFDSSDYEVLDSWGQGTQVTVSS |
| P_7 | SEQ ID NO: | MAEVQLQASGGGLVQAGGSLRLSCAAYGGTFNRYSMGWFRQAPGKEREFVARISWSVGSTKTYSDSV |
| (S-RBD) | 128 | KGRFTISRDNAKNTVYLQMNSLKPEDTAVYYCAAARVGEIIAVLISPSNYAYWGQGTQVTVSS |
| P_S12ß | SEQ ID NO: | MAEVQLVESGGGLVEAGGSLRLSCTTSGLTFSSVTMGWFRQAPGKEREFVAAIRWKFGNLGYADSVKG |
| (S-RBD) | 129 | RFTVSRDNAKNTVYLQMNSLKPEDTAVYYCAAARVGEIIAVLISPSNYAYWGQGTQVTVSS |
| P_F04-3ß | SEQ ID NO: 130 | MAEVQLVESGGGLVQAGGSLRLSCAASGRAFSRYFMGWFRQAPGKEREFVAGISRSGGSTDYANFVK |
| (S-RBD) | | GRFTISRDNAKNTVELQMNSLKPEDTAVYYCAATVDYSGTLTAARGREDYDDWGQGIQVTVSS |

The CDRs of the VHHs anti-S of Table 3 are given in the Table 4 below.

TABLE 4

| | VHH sequence | |
|---|---|---|
| | identification | |
| VHH Name | number | Amino acid sequence |
| P_S12 CDR1 | SEQ ID NO: 81 | GLTFSSVT |
| P_S12 CDR2 | SEQ ID NO: 82 | IRWKFGNLGY |
| P_S12 CDR3 | SEQ ID NO: 83 | AAARVGEIIAVLISPSNYAY |
| P_H08 CDR1 | SEQ ID NO: 84 | GSFFSISA |
| P_H08 CDR2 | SEQ ID NO: 85 | ITSGGSTNYA |
| P_H08 CDR3 | SEQ ID NO: 86 | HVQVGVHPIGYDV |
| P_S11 CDR1 | SEQ ID NO: 87 | GRTFSSLI |
| P_S11 CDR2 | SEQ ID NO: 88 | ITYSGGSTHY |
| P_S11 CDR3 | SEQ ID NO: 89 | AADTRGFSWSSSGGYDY |
| P_F04-3 CDR1 | SEQ ID NO: 131 | GRAFSRYF |
| P_F04-3 CDR2 | SEQ ID NO: 132 | ISRSGGSTDY |
| P_F04-3 CDR3 | SEQ ID NO: 133 | AATVDYSGTLTAARGREDYDD |
| P_G09-1 CDR1 | SEQ ID NO: 134 | GTGFTINA |
| P_G09-1 CDR2 | SEQ ID NO: 135 | ITRGDRIHYA |
| P_G09-1 CDR3 | SEQ ID NO: 136 | DVAAFDSSDYEVLDS |
| P_7 CDR1 | SEQ ID NO: 137 | GGTFNRYS |
| P_7 CDR2 | SEQ ID NO: 138 | ISWSVGSTKT |
| P_7 CDR3 | SEQ ID NO: 139 | AAARVGEIIAVLISPSNYAY |

Biological material deposits have been made at the CNCM, Institut Pasteur, Paris France. Specifically, the following VHH were deposited: VHH P_F04-3, VHH P_G09-1, VHH P_S12, VHH P_H08, and VHH P_S11.

E. coli comprising a vector coding for the VHH P_S11 (also named VHH S-NTD 11-2) was deposited, according to the Budapest Treaty, at CNCM (Collection Nationale de Cultures de Microorganismes, 25 rue du Docteur Roux, 75724 Paris Cedex 15, France) on Aug. 23, 2021 with the number I-5734.

E. coli comprising a vector coding for the VHH P_H08 (also named VHH S-NTD H08-4) was deposited, according to the Budapest Treaty, at CNCM (Collection Nationale de Cultures de Microorganismes, 25 rue du Docteur Roux, 75724 Paris Cedex 15, France) on Aug. 23, 2021 with the number I-5735.

E. coli comprising a vector coding for the VHH P_F04-3 (also named V S-RBD F04-3) was deposited, according to the Budapest Treaty, at CNCM (Collection Nationale de Cultures de Microorganismes, 25 rue du Docteur Roux, 75724 Paris Cedex 15, France) on Aug. 25, 2021 with the number I-5739.

E. coli comprising a vector coding for the VHH P_S12 (also named VHH S-RBD 12-4) was deposited, according to the Budapest Treaty, at CNCM (Collection Nationale de Cultures de Microorganismes, 25 rue du Docteur Roux, 75724 Paris Cedex 15, France) on Aug. 25, 2021 with the number I-5740.

E. coli comprising a vector coding for the VHH P_G09-1 (also named VHH-S-RBD G09-1) was deposited, according to the Budapest Treaty, at CNCM (Collection Nationale de Cultures de Microorganismes, 25 rue du Docteur Roux, 75724 Paris Cedex 15, France) on Aug. 25, 2021 with the number I-5741.

VHHs P_S11 and P_H08 recognize the NTD of S protein. The VHHs P_F04-3, P_F04-3β, P_S12, P_S12β and P_G09-1 recognize the RBD of S protein.

In an embodiment, the first and the second sdAbs, the first and the second sdAb being preferably VHHs, are not directed against the same epitope of S protein.

In an alternative embodiment, the first and the second sdAbs, the first and the second sdAb being preferably VHHs, are each one directed against the same epitope but the first and the second epitopes are from one different monomer among the three monomers constituting the native S protein.

The sdAb VHH P_S12 and VHH P_S12β bind to an epitope comprising at least one or two peptides comprising or consisting of amino acid sequence selected from the group consisting of SEQ ID NO: 164 (YNYLYRLF) and SEQ ID NO: 165 (VEGFNCYFPLQS) within region binding domain (RBD of SEQ ID NO: 163) of the S protein.

The sdAb VHH P_F04-3 and VHH P_F04-3β bind to a epitope comprising at least the peptide comprising or consisting of amino acid sequence SEQ ID NO: 166 (YNSASFSTFKCYGVSPT) within region binding domain (RBD of SEQ ID NO: 163) of the S protein.

The single domain VHH antibodies VHH P_G09-1 binds to a epitope comprising at least one, two, three or four peptides comprising or consisting of amino acid sequence selected from the group consisting of SEQ ID NO: 167 (RFASVYAWNR), SEQ ID NO: 169 (KVGGNYNYL), SEQ ID NO: 170 (RDIST) and SEQ ID NO: 171 (FPLQSYGFQP) within region binding domain (RBD of SEQ ID NO: 163) of the S protein and optionally the residue E at position 154 of the RDB of SEQ ID NO: 163.

Thus, in some embodiments, the first or second epitope is selected from the group consisting of:
an epitope comprising at least one or two peptides comprising or consisting of amino acid sequence selected from the group consisting of SEQ ID NO: 164 (YNYLYRLF) and SEQ ID NO: 165 (VEGFNCYFPLQS) within RBD of SEQ ID NO: 163,
an epitope comprising at least the peptide comprising or consisting of amino acid sequence SEQ ID NO: 166 (YNSASFSTFKCYGVSPT) within RBD of SEQ ID NO: 163,
an epitope comprising at least one, two, three or four peptides comprising or consisting of amino acid sequence selected from the group consisting of SEQ ID NO: 167 (RFASVYAWNR), SEQ ID NO: 169 (KVGGNYNYL), SEQ ID NO: 170 (RDIST) and SEQ ID NO: 171 (FPLQSYGFQP) within RBD of SEQ ID NO: 163 and optionally the residue E at position 154 of the RDB of SEQ ID NO: 163.

VHHs P_S12, P_H08, P_S11, P_F04-3, P_S12, P_S12β and P_G09-1 recognize S protein.

Thus, the first and/or the second sdAb may comprise:

### CDR1:

having the amino acid sequence selected from the group consisting of SEQ ID NO: 81, SEQ ID NO: 84, SEQ ID NO: 87, SEQ ID NO: 131, SEQ ID NO: 134 and SEQ ID NO: 137 and
variant thereof having up to two amino acids additions deletions and/or substitutions compared to amino acids sequences selected from the group consisting of SEQ ID NO: 81, SEQ ID NO: 84, SEQ ID NO: 87, SEQ ID NO: 131, SEQ ID NO: 134 and SEQ ID NO: 137

### CDR2:

having the amino acid sequence selected from the group consisting of SEQ ID NO: 82, SEQ ID NO: 85, SEQ ID NO: 88, SEQ ID NO: 132, SEQ ID NO: 135 and SEQ ID NO: 138 and
variant thereof having up to two amino acids additions deletions and/or substitutions compared to amino acids sequences selected from the group consisting of SEQ ID NO: 82, SEQ ID NO: 85, SEQ ID NO: 88, SEQ ID NO: 132, SEQ ID NO: 135 and SEQ ID NO: 138,

### CDR3:

having the amino acid sequence selected from the group consisting of SEQ ID NO: 83, SEQ ID NO: 86, SEQ ID NO: 89, SEQ ID NO: 133, SEQ ID NO: 136 and SEQ ID NO: 139 and
variant thereof having up to two amino acids additions deletions and/or substitutions compared to amino acids sequences selected from the group consisting of SEQ ID NO: 83, SEQ ID NO: 86 SEQ ID NO: 89, SEQ ID NO: 133, SEQ ID NO: 136 and SEQ ID NO: 139.

Thus, in the preferred embodiment where the first and/or the second sdAb are VHH, the first and/or the second VHH may comprise:

### CDR1:

having the amino acid sequence selected from the group consisting of SEQ ID NO: 81, SEQ ID NO: 84, SEQ ID NO: 87, SEQ ID NO: 131, SEQ ID NO: 134 and SEQ ID NO: 137 and
variant thereof having up to two amino acids additions deletions and/or substitutions compared to amino acids sequences selected from the group consisting of SEQ ID NO: 81, SEQ ID NO: 84, SEQ ID NO: 87, SEQ ID NO: 131, SEQ ID NO: 134 and SEQ ID NO: 137

### CDR2:

having the amino acid sequence selected from the group consisting of SEQ ID NO: 82, SEQ ID NO: 85, SEQ ID NO: 88, SEQ ID NO: 132, SEQ ID NO: 135 and SEQ ID NO: 138 and
variant thereof having up to two amino acids additions deletions and/or substitutions compared to amino acids sequences selected from the group consisting of SEQ ID NO: 82, SEQ ID NO: 85, SEQ ID NO: 88, SEQ ID NO: 132, SEQ ID NO: 135 and SEQ ID NO: 138,

### CDR3:

having the amino acid sequence selected from the group consisting of SEQ ID NO: 83, SEQ ID NO: 86, SEQ ID NO: 89, SEQ ID NO: 133, SEQ ID NO: 136 and SEQ ID NO: 139 and
variant thereof having up to two amino acids additions deletions and/or substitutions compared to amino acids sequences selected from the group consisting of SEQ ID NO: 83, SEQ ID NO: 86 SEQ ID NO: 89, SEQ ID NO: 133, SEQ ID NO: 136 and SEQ ID NO: 139.

In an embodiment, the first and/or the second sdAbs comprise:

CDR1, CDR2 and CDR3 having respectively the amino acid sequences selected from the groups:
SEQ ID NO: 81, SEQ ID NO: 82 and SEQ ID NO: 83,
SEQ ID NO: 84, SEQ ID NO: 85 and SEQ ID NO: 86,
SEQ ID NO: 87, SEQ ID NO: 88 and SEQ ID NO: 89,
SEQ ID NO: 131, SEQ ID NO: 132 and SEQ ID NO: 133,
SEQ ID NO: 134, SEQ ID NO: 135 and SEQ ID NO: 136,
SEQ ID NO: 137, SEQ ID NO: 138 and SEQ ID NO: 139.

In an embodiment, the first and/or the second VHHs comprise:

CDR1, CDR2 and CDR3 having respectively the amino acid sequences selected from the groups:
SEQ ID NO: 81, SEQ ID NO: 82 and SEQ ID NO: 83,
SEQ ID NO: 84, SEQ ID NO: 85 and SEQ ID NO: 86,
SEQ ID NO: 87, SEQ ID NO: 88 and SEQ ID NO: 89,
SEQ ID NO: 131, SEQ ID NO: 132 and SEQ ID NO: 133,
SEQ ID NO: 134, SEQ ID NO: 135 and SEQ ID NO: 136,
SEQ ID NO: 137, SEQ ID NO: 138 and SEQ ID NO: 139.

In an embodiment, the first and/or the second sdAbs comprises CDR1, CDR2 and CDR3 having respectively the amino acid sequences SEQ ID NO: 81, SEQ ID NO: 82 and SEQ ID NO: 83.

In an embodiment, the first and/or the second VHHs comprises CDR1, CDR2 and CDR3 having respectively the amino acid sequences SEQ ID NO: 81, SEQ ID NO: 82 and SEQ ID NO: 83.

In an embodiment, the first and/or the second sdAbs comprises CDR1, CDR2 and CDR3 having respectively the amino acid sequences SEQ ID NO: 84, SEQ ID NO: 85 and SEQ ID NO: 86.

In an embodiment, the first and/or the second VHHs comprises CDR1, CDR2 and CDR3 having respectively the amino acid sequences SEQ ID NO: 84, SEQ ID NO: 85 and SEQ ID NO: 86.

In an embodiment, the first and/or the second sdAbs comprises CDR1, CDR2 and CDR3 having respectively the amino acid sequences SEQ ID NO: 87, SEQ ID NO: 88 and SEQ ID NO: 89.

In an embodiment, the first and/or the second VHHs comprises CDR1, CDR2 and CDR3 having respectively the amino acid sequences SEQ ID NO: 87, SEQ ID NO: 88 and SEQ ID NO: 89.

Advantageously, the first and/or second VHH comprises:
an amino acid sequence selected from the group consisting of SEQ ID NO: 78, SEQ ID NO: 79, SEQ ID NO: 80, SEQ ID NO: 126, SEQ ID NO: 127, SEQ ID NO: 128, SEQ ID NO: 129 and SEQ ID NO: 130 or
an amino acid sequence that is at least 80%, at least 85%, at least 90%, at least 95%, at least 97% or at least 99% identical to an amino acid sequence selected from the group consisting of SEQ ID NO: 78, SEQ ID NO: 79 SEQ ID NO: 80, SEQ ID NO: 126, SEQ ID NO: 127, SEQ ID NO: 128, SEQ ID NO: 129 and SEQ ID NO: 130.

In an embodiment, the first and/or second VHH consists of:
an amino acid sequence selected from the group consisting of SEQ ID NO: 78, SEQ ID NO: 79 SEQ ID NO: 80, SEQ ID NO: 126, SEQ ID NO: 127, SEQ ID NO: 128, SEQ ID NO: 129 and SEQ ID NO: 130 or
an amino acid sequence that is at least 80%, at least 85%, at least 90%, at least 95%, at least 97% or at least 99% identical to an amino acid sequence selected from the group consisting of SEQ ID NO: 78, SEQ ID NO: 79 SEQ ID NO: 80, SEQ ID NO: 126, SEQ ID NO: 127, SEQ ID NO: 128, SEQ ID NO: 129 and SEQ ID NO: 130.

In a more preferred embodiment, the first and/or second VHH comprises an amino acid sequence selected from the group consisting of SEQ ID NO: 78, SEQ ID NO: 79, SEQ ID NO: 80, SEQ ID NO: 126, SEQ ID NO: 127, SEQ ID NO: 128, SEQ ID NO: 129 and SEQ ID NO: 130.

In a more preferred embodiment, the first and/or second VHH consist of an amino acid sequence selected from the group consisting of SEQ ID NO: 78, SEQ ID NO: 79 SEQ ID NO: 80, SEQ ID NO: 126, SEQ ID NO: 127, SEQ ID NO: 128, SEQ ID NO: 129 and SEQ ID NO: 130.

### Embodiment Wherein the Antigen is p24 of HIV

Examples of VHHs binding to P24 of HIV are given in Table 5 below.

TABLE 5

| | VHH | |
|---|---|---|
| | sequence | |
| VHH | identi- | |
| Name | fication | |
| (Target) | number | Amino acid sequence |
| 59H1 | 156 | MAQVQLVESGGGLVQAGGSLRLSCAASGSFFMSNVMAWYRQAP |
| (P24) | | GKARELIAAIRGGDMSTVYDDSVKGRFTITRDDDKNILYLQMNDLK |
| | | PEDTAMYYCKASGSSWGQGTQVTVSS |
| 2XV6_B | 157 | MADVQLKESGGGLVQAGGSLRLSCAASGSISRFNAMGWWRQAP |
| (also named | | GKEREFVARIVKGFDPVLADSVKGRFTISIDSAENTLALQMNRLKP |
| hereinafter | | EDTAVYYCFAALDTAYWGQGTQVTVSS |
| 2XV6, | | |
| unless | | |
| otherwise | | |
| specified) | | |
| (P24) | | |

These VHH are disclosed in the following articles: Gray, E. R., Brookes, J. C., Caillat, C., Turbe, V., Webb, B. L. J., Granger, L. A., Miller, B. S., McCoy, L. E., El Khattabi, M., Verrips, C. T., Weiss, R. A., Duffy, D. M., Weissenhorn, W., McKendry, R. A. Unravelling the Molecular Basis of High Affinity Nanobodies against HIV p24: In Vitro Functional, Structural, and in Silico Insights. (2017) ACS Infect Dis 3: 479-491. and Igonet, S., Vaney, M. C., Bartonova, V., Helma, J., Rothbauer, U., Leonhardt, H., Stura, E., Krausslich, H.-G., Rey, F. A. Targeting HIV-1 Virion Formation with Nanobodies—Implications for the Design of Assembly Inhibitors Published in the Protein databank: ID #2XV6 chain B and D.

The structure of both VHHs 59H1 and 2XV6_B have been co-crystallized with P24. The respective epitope of the two VHH have no intersection and far away from each other at least for avoiding any steric hindrance of the bound VHH.

In a preferred embodiment, the first and the second sdAbs, the first and the second sdAb being preferably VHHs, are not directed against the same epitope of P24.

Thus, in an embodiment, the first or second single domain antibody comprises the amino acid sequence SEQ ID NO: 156 or SEQ ID NO: 157 or an amino acid sequence that is at least 80%, at least 85%, at least 90%, at least 95%, at least 97% or at least 99% identical to an amino acid sequence SEQ ID NO: 156 or SEQ ID NO: 157.

In an embodiment, the first or second single domain antibody consists of the amino acid sequence SEQ ID NO: 156 or SEQ ID NO: 157 or an amino acid sequence that is at least 80%, at least 85%, at least 90%, at least 95%, at least 97% or at least 99% identical to an amino acid sequence SEQ ID NO: 156 or SEQ ID NO: 157.

In an embodiment, the first single domain antibody comprises the amino acid sequence 156 or an amino acid sequence that is at least 80%, at least 85%, at least 90%, at least 95%, at least 97% or at least 99% identical to an amino acid sequence 156 and the second single domain antibody comprises the amino acid sequence 157 or an amino acid sequence that is at least 80%, at least 85%, at least 90%, at least 95%, at least 97% or at least 99% identical to an amino acid sequence 157.

In an embodiment, the first single domain antibody consists of the amino acid sequence 156 or an amino acid sequence that is at least 80%, at least 85%, at least 90%, at least 95%, at least 97% or at least 99% identical to an amino acid sequence 156 and the second single domain antibody consists of the amino acid sequence 157 or an amino acid sequence that is at least 80%, at least 85%, at least 90%, at least 95%, at least 97% or at least 99% identical to an amino acid sequence 157.

In an embodiment, the first single domain antibody comprises the amino acid sequence 157 or an amino acid sequence that is at least 80%, at least 85%, at least 90%, at least 95%, at least 97% or at least 99% identical to an amino acid sequence 157 and the second single domain antibody comprises the amino acid sequence 156 or an amino acid sequence that is at least 80%, at least 85%, at least 90%, at least 95%, at least 97% or at least 99% identical to an amino acid sequence 156.

In an embodiment, the first single domain antibody consists of the amino acid sequence 157 or an amino acid sequence that is at least 80%, at least 85%, at least 90%, at least 95%, at least 97% or at least 99% identical to an amino acid sequence 157 and the second single domain antibody consists of the amino acid sequence 156 or an amino acid sequence that is at least 80%, at least 85%, at least 90%, at least 95%, at least 97% or at least 99% identical to an amino acid sequence 156.

### A.3. Luciferase and Fragments Thereof

According to the invention, the first fusion protein comprises a first fragment of a luciferase having:
the amino acid sequence as set forth in SEQ ID NO: 1 or
an amino acid sequence having at least 70%, at least 75%, at least 80%, at least 85%, at least 90%, at least 95%, at least 97% or at least 99% amino acid sequence identity to the amino acid sequence as set forth in SEQ ID NO: 1,
   and
the second fusion protein comprises a second fragment of a luciferase having:
   the amino acid sequence as set forth in SEQ ID NO: 2 or
an amino acid sequence having at least 70%, at least 75%, at least 80%, at least 85%, at least 90%, at least 95%, at least 97% or at least 99% amino acid sequence identity to the amino acid sequence as set forth in SEQ ID NO: 2.

Typically, the first and the second fragment of the luciferase have both no luciferase activity.

A luciferase activity can easily be assayed by a person skilled in the art. The luciferase activity of the fusion protein may be for example assayed with 8-(2,3-difluorobenzyl)-2-((5-methylfuran-2-yl)methyl)-6-phenylimidazo[1,2-a]pyrazin-3(7H)-one as substrate, a blank control and a positive control as for example the luciferase having the amino acid sequence SEQ ID NO: 3. The following percentage of relative luciferase activity may be calculated: [luminescence of the fusion protein−luminescence of the blank control]×100/luminescence of the positive control. If this percentage is negative, null or non-significant (e. g. lower than 10%, preferably than 5%, more preferably lower than 2.5%, most preferably lower than 1%), the person skilled in the art will consider that the fusion protein has no luciferase activity.

“Luciferase” as used herein refers to a class of oxidative enzymes that produce bioluminescence. Bioluminescence is the emission of light produced in a biochemical reaction involving the oxidation of a substrate via an enzyme. Luciferase is an enzyme emitting photon along the decarboxylation of a substrate, a luciferine.

“Identity” with respect to percent amino acid sequence “identity” for peptides and proteins is defined herein as the percentage of amino acid residues in the candidate sequence that are identical with the residues in the target sequences after aligning both sequences and introducing gaps, if necessary, to achieve the maximum percent sequence identity. Percent sequence identity is determined by conventional methods. Briefly, two amino acid sequences are aligned to optimize the alignment scores using the ClustalW algorithm (Thompson et al., Nuc. Ac. Res. 22:4673-4680, 1994) and PAM250 weight matrix (Dayhoff et al., “Atlas of Protein Sequence and Structure.” National Biomedical Research Foundation. Washington, DC 5:345-358, 1978) and default parameters as provided by the program MegAlign (DNASTAR, Inc.; Madison, WI). The percent identity is then calculated as: [Total number of identical matches×100]divided by [length of the longer sequence+number of gaps introduced into the longer sequence in order to align the two sequences].

The first fragment having the amino acid sequence as set forth in SEQ ID NO: 1 corresponds to amino acids 3-85 of the luciferase JAZ having the amino acid sequence as set forth in SEQ ID NO: 4.

The second fragment having the amino acid sequence as set forth in SEQ ID NO: 2 corresponds to amino acids 86-171 of the JAZ luciferase having the amino acid sequence as set forth in SEQ ID NO: 4.

JAZ luciferase is a mutant Y18R, L48K, Y116F, W134E, W163E and C166S of the KAZ/Nluc luciferase having the amino acid sequence SEQ ID NO. 3 and derived itself from the 19 kDa subunit of the luciferase from the deep-sea shrimp Oplophorus gracilirostris (Hall M P, Unch J, Binkowski B F, Valley M P, Butler B L, Wood M G, Otto P, Zimmerman K, Vidugiris G, Machleidt T, Robers M B, Benink H A, Eggers C T, Slater M R, Meisenheimer P L, Klaubert D H, Fan F, Encell L P, Wood K V. Engineered luciferase reporter from a deep sea shrimp utilizing a novel imidazopyrazinone substrate. ACS Chem Biol. 2012 Nov. 16; 7(11):1848-57).

Typically, the first fragment and the second fragment are both fragments of a luciferase. Each of these fragments have no luciferase activity by itself. However, when the first fragment is linked directly to the second fragment, the polypeptide constituted of the first and second fragments directly linked together has a luciferase activity.

The first and the second fragments of the luciferase having a similar size, it enables a better compensation of relative species and makes the dynamics of each fusion protein be equivalent. Moreover, such system has an intensity close to the one of the entire luciferase.

In an embodiment, the luciferase of which the first fragment and the second fragment are fragments is the JAZ luciferase or a mutant thereof. The first and the second fragments may be fragments of the same luciferase being JAZ luciferase or a mutant thereof or fragments of different luciferases among JAZ luciferase and mutant thereof. The amino acid sequences of the KAZ/Nluc luciferase, JAZ luciferase as well as mutants of JAZ luciferase are disclosed in the table 5 below.

TABLE 5

| Name | | |
|---|---|---|
| (substitution | | |
| compared to | SEQ | |
| KAZ/Nluc) | ID NO | Amino acid sequence |
| Amino acids 3- | SEQ ID | FTLEDFVGDWRQTAGRNLDQVLEQGGVSSLFQNLGVSVTPIQRIVKSGENGLKIDIHVIIPYEGLSGDQMG |
| 85 of JAZ | NO. 1 | QIEKIFKVVYPV |
| luciferase | | |
| (naJAZ) | | |
| Amino acids | SEQ ID | DDHHFKVILHYGTLVIDGVTPNMIDYFGRPFEGIAVFDGKKITVTGTLENGNKIIDERLINPDGSLLFRVT |
| 86-171 of JAZ | NO. 2 | INGVTGERLSERILA |
| luciferase | | |
| (noJAZ) | | |
| KAZ/Nluc | SEQ ID | MVFTLEDFVGDWRQTAGYNLDQVLEQGGVSSLFQNLGVSVTPIQRIVLSGENGLKIDIHVIIPYEGLSGDQ |
| | NO: 3 | MGQIEKIFKVVYPVDDHHFKVILHYGTLVIDGVTPNMIDYFGRPYEGIAVFDGKKITVTGTLWNGNKIIDER |
| | | LINPDGSLLFRVTINGVTGWRLCERILA |
| JAZ luciferase | SEQ ID | MVFTLEDFVGDWRQTAGRNLDQVLEQGGVSSLFQNLGVSVTPIQRIVKSGENGLKIDIHVIIPYEGLSGDQ |
| (Y18R, L48K, | NO. 4 | MGQIEKIFKVVYPVDDHHFKVILHYGTLVIDGVTPNMIDYFGRPFEGIAVFDGKKITVTGTLENGNKIIDER |
| Y116F, W134E, | | LINPDGSLLFRVTINGVTGERLSERILA |
| W163E and | | |
| C166S) | | |
| JAZ544 | SEQ ID | MVFTLEDFVGDWRQTAGYNLDQVLEQGGVSSLFQNLGVSVTPIQRIVLSGENGLKIDIHV |
| (Y116F) | NO: 5 | IIPYEGLSGDQMGQIEKIFKVVYPVDDHHFKVILHYGTLVIDGVTPNMIDYFGRPFEGIA |
| | | VFDGKKITVTGTLWNGNKIIDERLINPDGSLLFRVTINGVTGWRLCERILA |
| JAZ583 | SEQ ID | MVFTLEDFVGDWRQTAGYNLDQVLEQGGVSSLFQNLGVSVTPIQRIVLSGENGLKIDIHV |
| (W134T) | NO: 6 | IIPYEGLSGDQMGQIEKIFKVVYPVDDHHFKVILHYGTLVIDGVTPNMIDYFGRPYEGIA |
| | | VFDGKKITVTGTLTNGNKIIDERLINPDGSLLFRVTINGVTGWRLCERILA |
| JAZ584 | SEQ ID | MVFTLEDFVGDWRQTAGYNLDQVLEQGGVSSLFQNLGVSVTPIQRIVLSGENGLKIDIHV |
| (W163T) | NO: 7 | IPYEGLSGDQMGQIEKIFKVVYPVDDHHFKVILHYGTLVIDGVTPNMIDYFGRPYEGIA |
| | | VFDGKKITVTGTLWNGNKIIDERLINPDGSLLFRVTINGVTGTRLCERILA |
| JAZ560 | SEQ ID | MVFTLEDFVGDWRQTAGYNLDQVLEQGGVSSLFQNLGVSVTPIQRIVLSGENGLKADIHV |
| I56A | NO: 8 | IIPYEGLSGDQMGQIEKIFKVVYPVDDHHFKVILHYGTLVIDGVTPNMIDYFGRPYEGIA |
| | | VFDGKKITVTGTLWNGNKIIDERLINPDGSLLFRVTINGVTGWRLCERILA |
| JAZ585 | SEQ ID | MVFTLEDFVGDWRQTAGYNLDQVLEQGGVSSLFQNLGVSVTPIQRIVLSGENGLKIDIHV |
| (Y116F and | NO: 9 | IIPYEGLSGDQMGQIEKIFKVVYPVDDHHFKVILHYGTLVIDGVTPNMIDYFGRPFEGIA |
| W134T) | | VFDGKKITVTGTLTNGNKIIDERLINPDGSLLFRVTINGVTGWRLCERILA |
| JAZ619 | SEQ ID | MVFTLEDFVGDWRQTAGYNLDQVLEQGGVSSLFQNLGVSVTPIQRIVLSGENGLKIDIHV |
| (Y116F and | NO: 10 | IIPYEGLSGDQMGQIEKIFKVVYPVDDHHFKVILHYGTLVIDGVTPNMIDYFGRPFEGIA |
| C166S) | | VFDGKKITVTGTLWNGNKIIDERLINPDGSLLFRVTINGVTGWRLSERILA |
| JAZ536 | SEQ ID | MVFTLEDFVGDWRQTAGRNLDQVLEQGGVSSLFQNLGVSVTPIQRIVKSGENGLKIDIHV |
| (Y18R, L48K, | NO. 11 | IIPYEGLSGDQMGQIEKIFKVVYPVDDHHFKVILHYGTLVIDGVTPNMIDYFGRPYEGIA |
| W134E and | | VFDGKKITVTGTLENGNKIIDERLINPDGSLLFRVTINGVTGERLCERILA |
| W163E) | | |
| JAZ570 | SEQ ID | MVFTLEDFVGDWRQTAGRNLDQVLEQGGVSSLFQNLGVSVTPIQRIVKSGENGLKIDIHV |
| (Y18R, L48K, | NO: 12 | IIPYEGLSGDQMGQIEKIFKVVYPVDDHHFKVILHYGTLVIDGVTPNMIDYFGRPYEGIA |
| W134E, | | VFDGKKITVTGTLENGNKIIDERLINPDGSLLFRVTINGVTGERLSERILA |
| W163E and | | |
| C166S) | | |
| JAZ572 | SEQ ID | MVFTLEDFVGDWRQTAGRNLDQVLEQGGVSSLFQNLGVSVTPIQRIVKSGENGLKIDIHV |
| (Y18R, L48K, | NO: 13 | IIPYEGLSGDQMGQIEKIFKVVYPVDDHHFKVILHYGTLVIDGVTPNMIDYFGRPFEGIA |
| Y116F, | | VFDGKKITVTGTLENGNKIIDERLINPDGSLLFRVTINGVTGERLCERILA |
| W134E and | | |
| W163E) | | |
| JAZ*001 | SEQ ID | MVFTLEDFVGDWRQTAGRNLDQVLEQGGVSSLFQNLGVSVTPIQRIVLSGENGLKIDIHV |
| (Y18R) | NO: 14 | IIPYEGLSGDQMGQIEKIFKVVYPVDDHHFKVILHYGTLVIDGVTPNMIDYFGRPYEGIA |
| | | VFDGKKITVTGTLWNGNKIIDERLINPDGSLLFRVTINGVTGWRLCERILA |
| JAZ*002 | SEQ ID | MVFTLEDFVGDWRQTAGYNLDQVLEQGGVSSLFQNLGVSVTPIQRIVKSGENGLKIDIHV |
| (L48K) | NO: 15 | IIPYEGLSGDQMGQIEKIFKVVYPVDDHHFKVILHYGTLVIDGVTPNMIDYFGRPYEGIA |
| | | VFDGKKITVTGTLWNGNKIIDERLINPDGSLLFRVTINGVTGWRLCERILA |
| JAZ*003 | SEQ ID | MVFTLEDFVGDWRQTAGYNLDQVLEQGGVSSLFQNLGVSVTPIQRIVLSGENGLKADIHV |
| (I56A anc | NO: 16 | IIPYEGLSGDQMGQIEKIFKVVYPVDDHHFKVILHYGTLVIDGVTPNMIDYFGRPYEGIA |
| W163T) | | VFDGKKITVTGTLWNGNKIIDERLINPDGSLLFRVTINGVTGTRLCERILA |
| JAZ*004 | SEQ ID | MVFTLEDFVGDWRQTAGYNLDQVLEQGGVSSLFQNLGVSVTPIQRIVLSGENGLKIDIHV |
| (W134E) | NO: 17 | IIPYEGLSGDQMGQIEKIFKVVYPVDDHHFKVILHYGTLVIDGVTPNMIDYFGRPYEGIA |
| | | VFDGKKITVTGTLENGNKIIDERLINPDGSLLFRVTINGVTGWRLCERILA |
| JAZ*005 | SEQ ID | MVFTLEDFVGDWRQTAGYNLDQVLEQGGVSSLFQNLGVSVTPIQRIVLSGENGLKIDIHV |
| (W163E) | NO: 18 | IIPYEGLSGDQMGQIEKIFKVVYPVDDHHFKVILHYGTLVIDGVTPNMIDYFGRPYEGIA |
| | | VFDGKKITVTGTLWNGNKIIDERLINPDGSLLFRVTINGVTGERLCERILA |
| JAZ*006 | SEQ ID | MVFTLEDFVGDWRQTAGYNLDQVLEQGGVSSLFQNLGVSVTPIQRIVLSGENGLKIDIHV |
| (C166S) | NO: 19 | IIPYEGLSGDQMGQIEKIFKVVYPVDDHHFKVILHYGTLVIDGVTPNMIDYFGRPYEGIA |
| | | VFDGKKITVTGTLWNGNKIIDERLINPDGSLLFRVTINGVTGWRLSERILA |
| Amino acids | SEQ ID | DDHHFKVILHYGTLVIDGVTPNMIDYFGRPYEGIAVFDGKKITVTGTLENGNKIIDERLINPDGSLLFRVT |
| 86-171 of | NO: 114 | INGVTGERLSERILA |
| JAZ570 | | |
| Amino acids 3- | SEQ ID | FTLEDFVGDWRQTAGRNLDQVLEQGGVSSLFQNLGVSVTPIQRIVKSGENGLKIDIHVIIPYEGLLGDQMG |
| 85 of JAZ- | NO: 158 | QIEKIFKVVYPVL |
| L68S | | |
| luciferase | | |
| (L68S) | | |

Thus, in an embodiment, the luciferase has:
an amino acid sequence selected from the group consisting of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 and SEQ ID NO: 19 or
an amino acid sequence having at least 70%, at least 75%, at least 80%, at least 85%, at least 90%, at least 95%, at least 97% or at least 99% amino acid sequence identity to the amino acid sequence amino acid sequence selected from the group consisting of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 and SEQ ID NO: 19.

As used herein, reference to a luciferase shall be understood as including the variants of the luciferases as defined above.

A “variant” of a polypeptide (e.g., a sdAb, a VHH, or a luciferase) comprises an amino acid sequence wherein one or more amino acid residues are inserted into, deleted from and/or substituted into the amino acid sequence relative to another polypeptide sequence.

In an embodiment, the luciferase has an amino acid sequence selected from the group consisting of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 and SEQ ID NO: 19.

Preferably, the luciferase has the amino acid sequence selected from the group consisting of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 and SEQ ID NO: 19 or
an amino acid sequence having at least 70%, at least 75%, at least 80%, at least 85%, at least 90%, at least 95%, at least 97% or at least 99% amino acid sequence identity to the amino acid sequence amino acid sequence selected from the group consisting of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 and SEQ ID NO: 19.

In an embodiment, the luciferase has the amino acid sequence selected from the group consisting of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 and SEQ ID NO: 19.

In an embodiment, the luciferase has the amino acid sequence SEQ ID NO: 4 or SEQ ID NO: 12 or an amino acid sequence having at least 70%, at least 75%, at least 80%, at least 85%, at least 90%, at least 95%, at least 97% or at least 99% amino acid sequence identity to the amino acid sequence SEQ ID NO: 4 or SEQ ID NO: 12.

More preferably, the luciferase has the amino acid sequence SEQ ID NO: 4 or an amino acid sequence having at least 70%, at least 75%, at least 80%, at least 85%, at least 90%, at least 95%, at least 97% or at least 99% amino acid sequence identity to the amino acid sequence SEQ ID NO: 4.

In an embodiment, the luciferase has the amino acid sequence SEQ ID NO: 4.

In an embodiment, the first fragment consists:
in the amino acids 1, 2, 3, 4, 5, 6, 7, 8, 9 or 10 to the amino acids 75, 76, 77, 78, 79, 80, 81, 82, 83, 84, 85, 86, 87, 88, 89, 90, 91, 92, 93, 94 or 95 of the luciferase as defined above,
preferably in the amino acids 1, 2, 3, 4, 5, 6, 7 or 8 to the amino acids 80, 81, 82, 83, 84, 85, 86, 87, 88, 89 or 90 of the luciferase as defined above,
more preferably in the amino acids 1, 2, 3, 4, 5, 6, 7, 8 to the amino acids 80, 81, 82, 83, 84 or 85 of the luciferase as defined above,
most preferably in the amino acids 3 to 85 of the luciferase as defined above or variants thereof.

In an embodiment, the second fragment consists:
in the amino acids 76, 77, 78, 79, 80, 81, 82, 83, 84, 85, 86, 87, 88, 89, 90, 91, 92, 93, 94, 95 or 96 to the amino acids 151, 152, 153, 154, 155, 156, 57, 158, 159, 160, 161, 162, 163, 164, 165, 166, 167, 168, 169, 170 or 171 of the luciferase as defined above,
preferably in the amino acids 81, 82, 83, 84, 85, 86, 87, 88 or 89 to the amino acids 161, 162, 163, 164, 165, 166, 167, 168, 169, 170 or 171 of the luciferase as defined above,
more preferably in the amino acids 83, 84, 85, 86, 87, 88, 89, 90, 91 or 96 to the amino acids 163, 164, 165, 166, 167, 168, 169, 170 or 171 of the luciferase as defined above,
most preferably the amino acids 86 to 171 of the luciferase as defined above, or variants thereof.

In the fusion protein according to the invention, the fragment of luciferase is as defined above regarding the first and second fragments of a luciferase.

### A.4. Linker

Advantageously, the sdAb and the fragment of luciferase of the fusion protein are concatenated by a linker. Thus, the first sdAb and the first fragment of the luciferase may be concatenated by a linker, called first linker, and/or the second sdAb and the second fragment of the luciferase may be concatenated by a linker, called second linker. In the embodiment wherein the sdAb is a VHH, advantageously, the VHH and the fragment of luciferase of the fusion protein are concatenated by a linker. Thus, the first VHH and the first fragment of the luciferase may be concatenated by a linker, called first linker, and/or the second VHH and the second fragment of the luciferase may be concatenated by a linker, called second linker.

Linkers may be inserted in between the carboxy-terminal sequence of the VHH and the amino-terminal sequence of the fragment of luciferase.

As it is known by the person skilled in the art, the linker is chosen so as the reading frame of the C-term domain expression gene be kept and thus to keep unchanged the protein sequence of the C-terminal domain.

The size, the torque, the flexibility and the physical and chemical properties of the linker of each fusion protein is designed and screened for optimizing the spacing from target-bound sdAb and positioning for an optimal association required for recovering the luciferase catalytic activity.

Advantageously, the linker may monitor the distance, the orientation and/or the flexibility for optimizing the assembly of the two luciferase domains for the recovery of their activity. Thus, when the first and the second fusion proteins are bound to the same antigen entity, the two linkers allow a proper relative orientation and position of the two luciferase fragments that leads the luciferase catalytic activity recovery in the presence of substrates.

The linker of second fusion protein, called second linker, can be identical or different from the linker of the first fusion protein, called first linker.

Linker (first and/or second linker) may have an amino acid sequence from 1 to 90 residues, from 20 to 59 residues, from 23 to 45 residues from 35 to 65 residues or from 40 to 50 residues.

In an embodiment, the linker (first and/or second linker) comprises the amino acid sequence selected from the group consisting of G, GS, GₙSₚ with n=1 to 5 and p=1 to 3, SGnSp with n=1 to 5 and p=0 to 3, SEQ ID NO: 102, SEQ ID NO: 103, SEQ ID NO: 105 to SEQ ID NO: 108, SEQ ID NO: 110 to SEQ ID NO: 113, SEQ ID NO: 124 and SEQ ID NO: 140 to 154, or a variant thereof.

The amino acid sequences GₙSₚ with n=1 to 5 and p=1 to 3, SEQ ID NO: 102, SEQ ID NO: 103, SEQ ID NO: 105 to SEQ ID NO: 108 correspond to [GₙSₚ]q with n=1 to 5, p=1 to 3 and q=1 to 5 and the amino acid sequences SGnSp with n=1 to 5 and p=0 to 3, SEQ ID NO: 109 to 113 correspond to S[GₙSₚ] with n=1 to 5, p=0 to 3 and q=1 to 5 as disclosed in Table 6 below.

The variant of the linker may have at least 70%, at least 75%, at least 80%, at least 85%, at least 90%, at least 95%, at least 97% or at least 99% amino acid sequence identity to the amino acid sequence selected from the group consisting of G, GS, GₙSₚ with n=1 to 5 and p=1 to 3, SGnSp with n=1 to 5 and p=0 to 3, SEQ ID NO: 102, SEQ ID NO: 103, SEQ ID NO: 105 to SEQ ID NO: 108, SEQ ID NO: 110 to SEQ ID NO: 113 SEQ ID NO: 124 and SEQ ID NO: 140 to 154.

For example, variant of linker may have an amino acid sequence wherein one or more amino acid residues are inserted into, deleted from and/or substituted into the amino acid sequence relative to another linker.

In an embodiment, the linker (first and/or second linker) consists of the amino acid sequence selected from the group consisting of G, GS, GₙSₚ with n=1 to 5 and p=1 to 3, SGₙSₚ with n=1 to 5 and p=0 to 3, SEQ ID NO: 102, SEQ ID NO: 103, SEQ ID NO: 105 to SEQ ID NO: 108, SEQ ID NO: 110 to SEQ ID NO: 113 and SEQ ID NO: 124, SEQ ID NO: 140-154.

The amino acid sequence of examples of linkers are disclosed in the Table 6 below.

TABLE 6
Amino acid sequences of linkers

| | SEQ ID | |
|---|---|---|
| | NO: | Amino acid sequence |
| Linker 1 | 102 | AAAGEMETSQNPGEEKPQASPEGRPESETSCLVTTTDNQISTEQG |
| Linker 2 | 103 | AAAGEMETSQNPGEEKPQASPEGRPESETSCLVTTTDNQISTEQGAAAGEMETSQNPGEEKPQASPE |
| | | GRPESETSCLVTTTDNQISTEQG |
| Linker 3 | — | G |
| Linker 4 | — | GS |
| Linker 5 | — | GₙSₚ with n = 1 to 5 and p = 1 to 3 (equivalent to GXᵢXᵢXᵢXᵢSXⱼXⱼ with Xᵢ is G or nothing and Xⱼ is S or nothing) |
| Linker 6 | 105 | [GₙSₚ]_{q} with n = 1 to 5, p = 1 to 3 and q = 2 corresponding to GₙSₚ with n = 1 to 5 and p = 1 to 3 repeated 2 |
| | | times |
| Linker 7 | 106 | [GₙSₚ]_{q} with n = 1 to 5, p = 1 to 3 and q = 3 corresponding to GₙSₚ with n = 1 to 5 and p = 1 to 3 repeated 3 |
| | | times |
| Linker 8 | 107 | [GₙSₚ]_{q} with n = 1 to 5, p = 1 to 3 and q = 4 corresponding to GₙSₚ with n = 1 to 5 and p = 1 to 3 repeated 4 |
| | | times |
| Linker 9 | 108 | [GₙSₚ]_{q} with n = 1 to 5, p = 1 to 3 and q = 5 corresponding to GₙSₚ with n = 1 to 5 and p = 1 to 3 repeated 5 |
| | | times |
| Linker | — | SGₙSₚ with n =1 to 5 and p=0 to 3 (equivalent to SGXᵢXᵢXᵢXᵢSXⱼXⱼ with Xᵢ is G or nothing and Xⱼ is S or |
| 10 | | nothing) corresponding to S followed by GₙSₚ with n = 1 to 5 and p = 1 to 3 |
| Linker | 110 | S[GₙSₚ]_{q} with n = 1 to 5, p = 0 to 3 and q = 2 corresponding to S followed by GnSp with n = 1 to 5 and p = 1 to 3 |
| 11 | | repeated 2 times |
| Linker | 111 | S[GₙSₚ]_{q} with n = 1 to 5, p = 0 to 3 and q = 3 corresponding to S followed by GnSp with n = 1 to 5 and p = 1 to 3 |
| 12 | | repeated 3 times |
| Linker | 112 | S[GₙSₚ]_{q} with n = 1 to 5, p = 0 to 3 and q = 4 corresponding to S followed by GnSp with n = 1 to 5 and p = 1 to 3 |
| 13 | | repeated 4 times |
| Linker | 113 | S[GₙSₚ]_{q} with n = 1 to 5, p = 0 to 3 and q = 5 corresponding to S followed by GnSp with n = 1 to 5 and p = 1 to 3 |
| 14 | | repeated 5 times |
| Linker | 124 | LEVRSDKTHTCPPCP |
| 15 | | |
| Linker | 140 | AAAGEMETSQNPGEEKPQASPEGRPESETSTLVTTTDNQISTEQPGEEKPQASPEGRPG |
| 16 | | |
| Linker | 141 | AAAGEMETSQNPGEEKPQASPEGRPESETSTLVTTTDNQISTEQG |
| 17 | | |
| Linker | 142 | AAAGEMETSQNPGEEKPQASPEGRPESETSTLVTTTDNQISTE |
| 18 | | |
| Linker | 143 | AAAGEMETSQNPGEEKPQASPEGRPESETSTLVTTTDNQIS |
| 19 | | |
| Linker | 144 | AAAGEMETSQNPGEEKPQASPEGRPESETSTLVTTTDNQ |
| 20 | | |
| Linker | 145 | AAAGEMETSQNPGEEKPQASPEGRPESETSTLVTTTD |
| 21 | | |
| Linker | 146 | AAAGEMETSQNPGEEKPQASPEGRPESETSTLVTT |
| 22 | | |
| Linker | 147 | AAAGEMETSQNPGEEKPQASPEGRPESETSTLV |
| 23 | | |
| Linker | 148 | AAAGEMETSQNPGEEKPQASPEGRPESETST |
| 24 | | |
| Linker | 149 | AAAGEMETSQNPGEEKPQASPEGRPESET |
| 25 | | |
| Linker | 150 | AAAGEMETSQNPGEEKPQASPEGRPES |
| 26 | | |
| Linker | 151 | AAAGEMETSQNPGEEKPQASPEGRP |
| 27 | | |
| Linker | 152 | AAAGEMETSQNPGEEKPQASPEG |
| 28 | | |
| Linker | 153 | AAAGEMETSQNPGEEKPQASP |
| 29 | | |
| Linker | 154 | AAAGEMETSQNPGEEKPQAS |
| 30 | | |

In an embodiment, the linker is a derivative of the GS sequence. In this embodiment, the linker (first and/or second linker) may comprise or consist of the amino acid sequence selected from the group consisting of G, GS, GₙSₚ with n=1 to 5 and p=1 to 3, SGnSp with n=1 to 5 and p=0 to 3, SEQ ID NO: 105 to SEQ ID NO: 108 and SEQ ID NO: 110 to SEQ ID NO: 113 or a variant thereof.

In another embodiment, the linker is a derivative of the peptide having the amino acid sequence SEQ ID NO: 102. In this embodiment, the linker (first and/or second linker) may comprise or consist of the amino acid sequence selected from the group consisting of SEQ ID NO: 102, SEQ ID NO: 103 and SEQ ID NO: 140 to 154 or a variant thereof. In this embodiment the linker (first and/or second linker) may comprise or consist of the residues 1 to 20 (i.e. the amino acid sequence SEQ ID NO: 154), 21 (i.e. the amino acid sequence SEQ ID NO: 153), 22, 23 (i.e. the amino acid sequence SEQ ID NO: 152), 24, 25 (i.e. SEQ ID NO: 151), 26, 27 (i.e. the amino acid sequence SEQ ID NO: 150), 28, 29 (i.e. the amino acid sequence SEQ ID NO: 149), 30, 31 (i.e. the amino acid sequence SEQ ID NO: 148), 32, 33 (i.e. the amino acid sequence SEQ ID NO: 147), 34, 35 (i.e. the amino acid sequence SEQ ID NO: 146), 36, 37 (i.e. the amino acid sequence SEQ ID NO: 145), 38, 39 (i.e. the amino acid sequence SEQ ID NO: 144), 40, 41 (i.e. the amino acid sequence SEQ ID NO: 143), 42, 43 (i.e. the amino acid sequence SEQ ID NO: 142), 44, 45, 46, 47, 48, 49, 50, 51, 52, 53, 54, 55, 56, 57, 58 or 59 of SEQ ID NO: 140 or a variant thereof.

The present invention also relates to a linker comprising or consisting of the amino acid sequence selected from the group consisting of SEQ ID NO: 102, SEQ ID NO: 103 and SEQ ID NO: 140 to 154 or a variant thereof. In an embodiment the linker may comprise or consist of the residues 1 to 20 (i.e. the amino acid sequence SEQ ID NO: 154), 21 (i.e. the amino acid sequence SEQ ID NO: 153), 22, 23 (i.e. the amino acid sequence SEQ ID NO: 152), 24, 25 (i.e. SEQ ID NO: 151), 26, 27 (i.e. the amino acid sequence SEQ ID NO: 150), 28, 29 (i.e. the amino acid sequence SEQ ID NO: 149), 30, 31 (i.e. the amino acid sequence SEQ ID NO: 148), 32, 33 (i.e. the amino acid sequence SEQ ID NO: 147), 34, 35 (i.e. the amino acid sequence SEQ ID NO: 146), 36, 37 (i.e. the amino acid sequence SEQ ID NO: 145), 38, 39 (i.e. the amino acid sequence SEQ ID NO: 144), 40, 41 (i.e. the amino acid sequence SEQ ID NO: 143), 42, 43 (i.e. the amino acid sequence SEQ ID NO: 142), 44, 45, 46, 47, 48, 49, 50, 51, 52, 53, 54, 55, 56, 57, 58 or 59 of SEQ ID NO: 140 or a variant thereof.

The present invention relates to a method for selecting a linker, preferably for selecting a linker from linkers comprising or consisting of the amino acid sequence selected from the group consisting of SEQ ID NO: 102, SEQ ID NO: 103 and SEQ ID NO: 140 to 154 or a variant thereof.

This method comprises a step (a) of producing:
at least one, two, three or four, preferably four, first fusion proteins comprising or consisting of:
   a N-terminal domain which comprises a single domain antibody wherein the single domain antibody is selected from:
      a single domain antibody (VHHₑₚ₁) which is directed against a first epitope of a given antigen or
      a single domain antibody (VHHₑₚ₂) which is directed against a second epitope of said antigen,
   a C-terminal domain which consists of a first fragment of a luciferase (F1) as defined in section A.3 above
   and
   a linker linking the single domain antibody to the first fragment of a luciferase wherein the single domain antibody is selected from:
      a linker called short linker (L_{S}), having the amino acid sequence consisting in the residues 1 to 20, 21, 22 or 23 of SEQ ID NO: 140 or variant thereof
      or
      a linker called long linker (L_{L}), having the residues 1 to 45, 46, 47, 48, 49, 50, 51, 52, 53, 54, 55, 56, 57, 58 or 59 of SEQ ID NO: 140 or variant thereof and
at least one, two, three or four, preferably four, second fusion proteins comprising or consisting in:
   a N-terminal domain which comprises a single domain antibody wherein the single domain antibody is selected from:
      the single domain antibody (VHHₑₚ₁) which is directed against a first epitope a given antigen or
      the single domain antibody (VHH₂) which is directed against a second epitope of said antigen,
   a C-terminal domain which consists of a second fragment (F2) of a luciferase, wherein the second fragment of a luciferase is as defined in section A.3 above
   and
   a linker linking the single domain antibody to the second fragment of a luciferase wherein the single domain antibody is selected from:
   a linker, called short linker (L_{S}), having the amino acid sequence consisting in the residues 1 to 20, 21, 22 or 23 of SEQ ID NO: 140 or variant thereof
   or
   a linker called long linker (L_{L}), having consisting in the residues 1 to 45, 46, 47, 48, 49, 50, 51, 52, 53, 54, 55, 56, 57, 58 or 59 of SEQ ID NO: 140 or variant thereof.

Schematically, the at least one, two, three or four first fusion proteins are selected from the group consisting of VHHₑₚ₁-L_{S}-F1, VHHₑₚ₁-L_{L}-F1, VHHₑₚ₂-L_{S}-F1 and VHHₑₚ₂-L_{L}-F1 and the at least one, two, three or four second fusion proteins are selected from the group consisting of VHHₑₚ₁-L_{S}-F2, VHHₑₚ₁-L_{L}-F2, VHHₑₚ₂-L_{S}-F2 and VHHₑₚ₂-L_{L}-F2.

In some preferred embodiments:
the first fragment of luciferase has the amino acid sequence SEQ ID NO: 1 or a variant thereof, for example a variant having an amino acid sequence wherein one, two or three amino acid residues are inserted into, deleted from and/or substituted into,
the second fragment of luciferase has the amino acid sequence SEQ ID NO: 2 or a variant thereof, for example a variant having an amino acid sequence wherein one, two or three amino acid residues are inserted into, deleted from and/or substituted into,
the short linker has the amino acid sequence SEQ ID NO: 152 or a variant thereof, for example a variant having an amino acid sequence wherein one, two or three amino acid residues are inserted into, deleted from and/or substituted into,
the long linker has the amino acid sequence SEQ ID NO: 140, 102 or 141 or a variant thereof, for example a variant having an amino acid sequence wherein one, two or three amino acid residues are inserted into, deleted from and/or substituted into.

In some more preferred embodiments, the first fragment of luciferase has the amino acid sequence SEQ ID NO: 1, the second fragment of luciferase has the amino acid sequence SEQ ID NO: 2, the short linker has the amino acid sequence SEQ ID NO: 152 and the long linker has the amino acid sequence SEQ ID NO: 140, 102 or 141.

This method also comprises the steps of:
(b) for one of the at least two, three, four, five, six, seven or eight, preferably eight system comprising one of the first fusion protein and one of the second fusion protein wherein:
if the single domain antibody of the first fusion protein is the single domain antibody which is directed against the first epitope of the antigen, the single domain antibody of the second fusion protein is the single domain antibody which is directed against the second epitope of the antigen
   α) contacting:
      a sample comprising the antigen,
      a substrate of the luciferase and
      the system,
   and
   β) quantifying the luminescence,
(c) repeating step (b) for at least one, two, three, four, five, six or seven of the other system.

The step (b) may comprise a step (γ) of comparing the quantified luminescence with the one of a blank control (i.e. without antigen).

The method comprises a step of selecting among the systems of which luminescence has been quantified, the system wherein the luminescence is the highest, preferably compared to the blank control (ratio without antigen/without antigen).

The method may also comprise an additional step wherein if the highest luminescence is obtained with the first and/or second fusion protein with the long linker, the long linker is shortened of one or two residues in the corresponding first and/or second fusion protein and the luminescence in presence of the sample comprising the antigen and the substrate is quantified, this step is repeated until the luminescence reaches its optimal. Preferably, the linker must not be shorter than the linker having the amino acid sequence SEQ ID NO: 154.

In the same way, the method may also comprise an additional step wherein if the highest luminescence is obtained with the first and/or second fusion protein with the short linker, the short linker is extended of one or two residues, the one or two residues corresponding to the residues of the amino acid sequence SEQ ID NO: 140 and the luminescence in presence of the sample comprising the antigen and the substrate is quantified, this step is repeated until the luminescence reaches its optimal.

Preferably, the linker must not be longer than the linker having the amino acid sequence SEQ ID NO: 140.

In an alternative embodiment, the fusion protein (first and/or second fusion proteins) may comprise no linker.

### A.5. Heterologous Amino Acid Sequences at the N-Terminus, C-Terminus

The fusion protein (first fusion protein and/or second fusion protein) of the invention may have one or more heterologous amino acid sequences at the N-terminus, C-terminus, or both. The heterologous sequence may be for example a signal peptide, a tag, such as a tag for purification purpose.

An example of signal peptide is the signal peptide having the amino acid sequence SEQ ID NO: 123.

Affinity tags may be used at the C-end of the fragment of luciferase amino-acid sequence for purification, for secondary binding probe, for bead binding, for solid substrate binding purpose. Examples of amino acid sequence of such tags are given in the Table 7 below.

TABLE 7

| | SEQ ID | |
|---|---|---|
| Name | NO | Amino acid sequence |
| HIS-TAG | SEQ ID | HHHHHH |
| | NO: 60 | |
| AviTAG | SEQ ID | GLNDIFEAQKIEWHE |
| | NO: 61 | |
| SBP 37 | SEQ ID | DEKTTGWRGGHVVEGLAGELEQLRARLEHHPQGQREP |
| | NO: 62 | |
| Twin- | SEQ ID | WSHPQFEKGGGSGGGSGGSAWSHPQFEK |
| Strep-Tag | NO: 63 | |
| HA-Tag | SEQ ID | YPYDVPDYA |
| | NO: 64 | |
| MYC-Tag | SEQ ID | EQKLISEEDL |
| | NO: 65 | |

The tag may be preceded by the sequence LE.

The N-terminal methionine may be followed by another amino acid, for example an alanine.

### Embodiments of Fusion Proteins

Some examples of fusion protein are given below. Such fusion protein may comprise from its amino-end to its carboxy-end: a heterologous amino acid sequence at its amino terminal end (e.g. MA), a sequence of a sdAb, preferably a VHH, directed against an epitope of an antigen, a sequence of a linker (e.g. linker of SEQ ID NO: 102), a sequence of a fragment of a luciferase (e.g. for the first fusion protein: fragment having SEQ ID NO: 1 corresponding to amino acids 3-85 of the JAZ luciferase having SEQ ID NO: 4 and for the second fusion protein fragment having SEQ ID NO: 2 corresponding to amino acids 86-171 of the JAZ luciferase having SEQ ID NO: 4) and a heterologous amino acid sequence at its carboxy terminal end (e.g. LE followed by an histidine tag of SEQ ID NO: 60).

### Embodiments Wherein the Antigen is the N Protein of SARS-CoV-2

It is exemplified below a first fusion protein (VHH677-naJAZ) having the amino acid sequence SEQ ID NO: 66 and second fusion proteins (VHH690-noJAZ, VHH690-noJAZ570) having respectively the amino acid sequence SEQ ID NO: 67 and SEQ ID NO: 70. These fusion proteins are suitable to be used in a system for detecting N protein, preferably N protein of SARS-CoV-2.

VHH677-naJAZ comprises amino acids MA at its N-terminal end, amino acid sequence SEQ ID NO: 23 of VHH G9-1, a linker having the amino acid sequence SEQ ID NO: 102, a first fragment having SEQ ID NO: 1 (corresponding to amino acids 3-85 of the JAZ luciferase having SEQ ID NO: 4), amino acids LE followed by an histidine tag of SEQ ID NO: 60.

VHH690-noJAZ comprises amino acids MA at its N-terminal end, amino acid sequence SEQ ID NO: 25 of VHH C7-1, a linker having the amino acid sequence SEQ ID NO: 102, a first fragment having SEQ ID NO: 2 (corresponding to amino acids 86-171 of the JAZ luciferase having SEQ ID NO: 4), amino acids LE followed by an histidine tag of SEQ ID NO: 60.

VHH690-noJAZ570 corresponds to VHH690-noJAZ wherein the first fragment having SEQ ID NO: 2 has been replaced with the first fragment having SEQ ID NO: 114 (corresponding to amino acids 86-171 of the JAZ570 luciferase having SEQ ID NO: 12). Different combinations are possible. For example, VHH690-naJAZ as first fusion protein and VHH677-noJAZ as second fusion protein may an alternative combination to VHH677-naJAZ as first fusion protein and VHH690-noJAZ as second fusion protein.

TABLE 8

| | SEQ | |
|---|---|---|
| | ID | |
| Name | NO | Amino acid sequence |
| VHH677-naJAZ | 66 | MAEVQLVESGGGLVEPGGSLRLSCAASGFTWDYYDIGWFRQAPGKEREGVACISSSGSSTNYGDSVKGRFTI |
| | | SRDNAKKTVYLQMNSLKPEDTAVYYCAADIVDYGLESASCMWIDRGYWGQGTQVTVSSAAAGEMETSQNPG |
| | | EEKPQASPEGRPESETSCLVTTTDNQISTEQGFTLEDFVGDWRQTAGRNLDQVLEQGGVSSLFQNLGVSVTPI |
| | | QRIVKSGENGLKIDIHVIIPYEGLSGDQMGQIEKIFKVVYPVLEHHHHHH |
| VHH690-noJAZ | 67 | MAEVQLQASGGGLVQPGGSLRLSCAASGFTLGYYRIGWFRQAPGKEREGVSCLSSSGRSTNYADSVKGRFTI |
| | | STDNAKNTVYLQMDSLKPEDTAVYYCAADFTPGPRLCSILSLNEYSAWGQGTQVTVSSAAAGEMETSQNPGE |
| | | EKPQASPEGRPESETSCLVTTTDNQISTEQGDDHHFKVILHYGTLVIDGVTPNMIDYFGRPFEGIAVEDGKKIT |
| | | VTGTLENGNKIIDERLINPDGSLLFRVTINGVTGERLSERILALEHHHHHH |
| VHH677-naJAZ | 68 | EVQLVESGGGLVEPGGSLRLSCAASGFTWDYYDIGWFRQAPGKEREGVACISSSGSSTNYGDSVKGRFTISR |
| without N and | | DNAKKTVYLQMNSLKPEDTAVYYCAADIVDYGLESASCMWIDRGYWGQGTQVTVSSAAAGEMETSQNPGEE |
| C terminal | | KPQASPEGRPESETSCLVTTTDNQISTEQGFTLEDFVGDWRQTAGRNLDQVLEQGGVSSLFQNLGVSVTPIQ |
| sequences | | RIVKSGENGLKIDIHVIIPYEGLSGDQMGQIEKIFKVVYPV |
| VHH690-noJAZ | 69 | EVQLQASGGGLVQPGGSLRLSCAASGFTLGYYRIGWFRQAPGKEREGVSCLSSSGRSTNYADSVKGRFTIST |
| without N and | | DNAKNTVYLQMDSLKPEDTAVYYCAADFTPGPRLCSILSLNEYSAWGQGTQVTVSSAAAGEMETSQNPGEEK |
| C terminal | | PQASPEGRPESETSCLVTTTDNQISTEQGDDHHFKVILHYGTLVIDGVTPNMIDYEGRPIEGIAVEDGKKITVT |
| sequences | | GTLENGNKIIDERLINPDGSLLFRVTINGVTGERLSERILA |
| VHH690- | 70 | MAEVQLQASGGGLVQPGGSLRLSCAASGFTLGYYRIGWFRQAPGKEREGVSCLSSSGRSTNYADSVKGRFTI |
| noJAZ570 | | STDNAKNTVYLQMDSLKPEDTAVYYCAADFTPGPRLCSILSLNEYSAWGQGTQVTVSSAAAGEMETSQNPGE |
| | | EKPQASPEGRPESETSCLVTTTDNQISTEQGDDHHFKVILHYGTLVIDGVTPNMIDYEGRPYEGIAVEDGKKIT |
| | | VTGTLENGNKIIDERLINPDGSLLFRVTINGVTGERLSERILALEHH |
| VHH690- | 71 | EVQLQASGGGLVQPGGSLRLSCAASGFTLGYYRIGWFRQAPGKEREGVSCLSSSGRSTNYADSVKGRFTIST |
| noJAZ570 | | DNAKNTVYLQMDSLKPEDTAVYYCAADFTPGPRLCSILSLNEYSAWGQGTQVTVSSAAAGEMETSQNPGEEK |
| without N and | | PQASPEGRPESETSCLVTTTDNQISTEQGDDHHEKVILHYGTLVIDGVTPNMIDYEGRPYEGIAVEDGKKITVT |
| C terminal | | GTLENGNKIIDERLINPDGSLLFRVTINGVTGERLSERILA |
| sequences | | |
| VHH690-naJAZ | 72 | MAEVQLQASGGGLVQPGGSLRLSCAASGFTLGYYRIGWFRQAPGKEREGVSCLSSSGRSTNYADSVKGRFTI |
| | | STDNAKNTVYLQMDSLKPEDTAVYYCAADFTPGPRLCSILSLNEYSAWGQGTQVTVSSAAAGEMETSQNPGE |
| | | EKPQASPEGRPESETSCLVTTTDNQISTEQGFTLEDFVGDWRQTAGRNLDQVLEQGGVSSLFQNLGVSVTPI |
| | | QRIVKSGENGLKIDIHVIIPYEGLSGDQMGQIEKIFKVVYPVLEHHHHHH |
| VHH677-noJAZ | 73 | MAEVQLVESGGGLVEPGGSLRLSCAASGFTWDYYDIGWFRQAPGKEREGVACISSSGSSTNYGDSVKGRFTI |
| | | SRDNAKKTVYLQMNSLKPEDTAVYYCAADIVDYGLESASCMWIDRGYWGQGTQVTVSSAAAGEMETSQNPG |
| | | EEKPQASPEGRPESETSCLVTTTDNQISTEQGDDHHFKVILHYGTLVIDGVTPNMIDYFGRPFEGIAVEDGKKI |
| | | TVTGTLENGNKIIDERLINPDGSLLFRVTINGVTGERLSERILALEHHHHHH |
| VHH690-naJAZ | 74 | EVQLQASGGGLVQPGGSLRLSCAASGFTLGYYRIGWFRQAPGKEREGVSCLSSSGRSTNYADSVKGRFTIST |
| without N and | | DNAKNTVYLQMDSLKPEDTAVYYCAADFTPGPRLCSILSLNEYSAWGQGTQVTVSSAAAGEMETSQNPGEEK |
| C terminal | | PQASPEGRPESETSCLVTTTDNQISTEQGFTLEDFVGDWRQTAGRNLDQVLEQGGVSSLFQNLGVSVTPIQRI |
| sequences | | VKSGENGLKIDIHVIIPYEGLSGDQMGQIEKIFKVVYPV |
| VHH677-noJAZ | 75 | EVQLVESGGGLVEPGGSLRLSCAASGFTWDYYDIGWFRQAPGKEREGVACISSSGSSTNYGDSVKGRFTISR |
| without N and | | DNAKKTVYLQMNSLKPEDTAVYYCAADIVDYGLESASCMWIDRGYWGQGTQVTVSSAAAGEMETSQNPGEE |
| C terminal | | KPQASPEGRPESETSCLVTTTDNQISTEQGDDHHFKVILHYGTLVIDGVTPNMIDYFGRPFEGIAVEDGKKITV |
| sequences | | TGTLENGNKIIDERLINPDGSLLFRVTINGVTGERLSERILA |
| VHH677- | 76 | MAEVQLVESGGGLVEPGGSLRLSCAASGFTWDYYDIGWFRQAPGKEREGVACISSSGSSTNYGDSVKGRFTI |
| noJAZ570 | | SRDNAKKTVYLQMNSLKPEDTAVYYCAADIVDYGLESASCMWIDRGYWGQGTQVTVSSAAAGEMETSQNPG |
| | | EEKPQASPEGRPESETSCLVTTTDNQISTEQGDDHHFKVILHYGTLVIDGVTPNMIDYEGRPYEGIAVEDGKKI |
| | | TVTGTLENGNKIIDERLINPDGSLLFRVTINGVTGERLSERILALEHHHHHH |
| VHH677- | 77 | EVQLVESGGGLVEPGGSLRLSCAASGFTWDYYDIGWFRQAPGKEREGVACISSSGSSTNYGDSVKGRFTISR |
| noJAZ570 | | DNAKKTVYLQMNSLKPEDTAVYYCAADIVDYGLESASCMWIDRGYWGQGTQVTVSSAAAGEMETSQNPGEE |
| without N and | | KPQASPEGRPESETSCLVTTTDNQISTEQGDDHHFKVILHYGTLVIDGVTPNMIDYFGRPYEGIAVFDGKKITV |
| C terminal | | TGTLENGNKIIDERLINPDGSLLFRVTINGVTGERLSERILA |
| sequences | | |
| VHH648-JAZ | 119 | MAEVQLVESGGGLVEPGGSLRLSCAASGFTWDYYDIGWFRQAPGKEREGVACISSSGSSTNYGDSVKGRFTI |
| (P_G9) | | SRDNAKKTVYLQMNSLKPEDTAVYYCAADIVDYGLESASCMWIDRGYWGQGTQVTVSSAAAVFTLEDFVGDW |
| | | RQTAGRNLDQVLEQGGVSSLFQNLGVSVTPIQRIVKSGENGLKIDIHVIIPYEGLSGDQMGQIEKIFKVVYPVD |
| | | DHHFKVILHYGTLVIDGVTPNMIDYFGRPYEGIAVFDGKKITVTGTLENGNKIIDERLINPDGSLLFRVTINGV |
| | | TGERLCERILALEHHHHHH |
| VHH655-SBP37 | 120 | MAEVQLVESGGGLVEPGGSLRLSCAASGFTWDYYDIGWFRQAPGKEREGVACISSSGSSTNYGDSVKGRFTI |
| (P_E4.3) | | SRDNAKKTVYLQMNSLKPEDTAVYYCAADIVDYGLESASCMWIDRGYWGQGTQVTVSSAAADEKTTGWRGG |
| | | HVVEGLAGELEQLRARLEHHPQGQREPLEHHHHHH |
| VHH anti-N-Fc- | 121 | MYRMQLLSCIALSLALVTNSASMAEVQLVESGGGLVEPGGSLRLSCAASGFTWDYYDIGWFRQAPGKEREGV |
| IgG1 | | ACISSSGSSTNYGDSVKGRFTISRDNAKKTVYLQMNSLKPEDTAVYYCAADIVDYGLESASCMWIDRGYWGQG |
| (G9.1) | | TQVTVSSLEVRSDKTHTCPPCPAPELLGGPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNKALPAPIEKTISKAKGQPREPQVYTLPPS |
| | | RDELTKNQVSLTCLVKGFYPSDIAVEWESNGQPENNYKTTPPVLDSDGSFFLYSKLTVDKSRWQQGNVFSCS |
| | | VMHEALHNHYTQKSLSLSPGKHHHHHHV |

In an embodiment, the first fusion protein comprises the amino acid sequence SEQ ID NO: 68 or an amino acid sequence having at least 70%, at least 75%, at least 80%, at least 85%, at least 90%, at least 95%, at least 97% or at least 99% amino acid sequence identity thereof.

In an embodiment, the second fusion protein comprises the amino acid sequence SEQ ID NO: 69 or SEQ ID NO: 71 or an amino acid sequence having at least 70%, at least 75%, at least 80%, at least 85%, at least 90%, at least 95%, at least 97% or at least 99% amino acid sequence identity thereof.

In an embodiment, the first fusion protein comprises the amino acid sequence SEQ ID NO: 68 and the second fusion protein comprises the amino acid sequence SEQ ID NO: 69 or SEQ ID NO: 71 or an amino acid sequence having at least 70%, at least 75%, at least 80%, at least 85%, at least 90%, at least 95%, at least 97% or at least 99% amino acid sequence identity thereof.

In an embodiment, the first fusion protein comprises the amino acid sequence SEQ ID NO: 66 or an amino acid sequence having at least 70%, at least 75%, at least 80%, at least 85%, at least 90%, at least 95%, at least 97% or at least 99% amino acid sequence identity thereof.

In an embodiment, the second fusion protein comprises the amino acid sequence SEQ ID NO: 67 or SEQ ID NO: 70 or an amino acid sequence having at least 70%, at least 75%, at least 80%, at least 85%, at least 90%, at least 95%, at least 97% or at least 99% amino acid sequence identity thereof.

In an embodiment, the first fusion protein comprises the amino acid sequence SEQ ID NO: 66 or an amino acid sequence having at least 70%, at least 75%, at least 80%, at least 85%, at least 90%, at least 95%, at least 97% or at least 99% amino acid sequence identity thereof and the second fusion protein comprises the amino acid sequence SEQ ID NO: 67 or SEQ ID NO: 70 or an amino acid sequence having at least 70%, at least 75%, at least 80%, at least 85%, at least 90%, at least 95%, at least 97% or at least 99% amino acid sequence identity thereof.

In an embodiment, the first fusion protein consists of the amino acid sequence SEQ ID NO: 66 or an amino acid sequence having at least 70%, at least 75%, at least 80%, at least 85%, at least 90%, at least 95%, at least 97% or at least 99% amino acid sequence identity thereof.

In an embodiment, the second fusion protein consists of the amino acid sequence SEQ ID NO: 67 or SEQ ID NO: 70 or an amino acid sequence having at least 70%, at least 75%, at least 80%, at least 85%, at least 90%, at least 95%, at least 97% or at least 99% amino acid sequence identity thereof.

In an embodiment, the first fusion protein consists of the amino acid sequence SEQ ID NO: 66 or an amino acid sequence having at least 70%, at least 75%, at least 80%, at least 85%, at least 90%, at least 95%, at least 97% or at least 99% amino acid sequence identity thereof and the second fusion protein consists of the amino acid sequence SEQ ID NO: 67 or SEQ ID NO: 70 or an amino acid sequence having at least 70%, at least 75%, at least 80%, at least 85%, at least 90%, at least 95%, at least 97% or at least 99% amino acid sequence identity thereof.

In an alternative embodiment, the first fusion protein comprises the amino acid sequence SEQ ID NO: 74 or an amino acid sequence having at least 70%, at least 75%, at least 80%, at least 85%, at least 90%, at least 95%, at least 97% or at least 99% amino acid sequence identity thereof.

In an alternative embodiment, the second fusion protein comprises the amino acid sequence SEQ ID NO: 75 or SEQ ID NO: 77 or an amino acid sequence having at least 70%, at least 75%, at least 80%, at least 85%, at least 90%, at least 95%, at least 97% or at least 99% amino acid sequence identity thereof.

In an alternative embodiment, the first fusion protein comprises the amino acid sequence SEQ ID NO: 74 or an amino acid sequence having at least 70%, at least 75%, at least 80%, at least 85%, at least 90%, at least 95%, at least 97% or at least 99% amino acid sequence identity thereof and the second fusion protein comprises the amino acid sequence SEQ ID NO: 75 or SEQ ID NO: 77 or an amino acid sequence having at least 70%, at least 75%, at least 80%, at least 85%, at least 90%, at least 95%, at least 97% or at least 99% amino acid sequence identity thereof.

In an alternative embodiment, the first fusion protein comprises the amino acid sequence SEQ ID NO: 72 or an amino acid sequence having at least 70%, at least 75%, at least 80%, at least 85%, at least 90%, at least 95%, at least 97% or at least 99% amino acid sequence identity thereof.

In an alternative embodiment, the second fusion protein comprises the amino acid sequence SEQ ID NO: 73 or SEQ ID NO: 76 or an amino acid sequence having at least 70%, at least 75%, at least 80%, at least 85%, at least 90%, at least 95%, at least 97% or at least 99% amino acid sequence identity thereof.

In an alternative embodiment, the first fusion protein comprises the amino acid sequence SEQ ID NO: 72 or an amino acid sequence having at least 70%, at least 75%, at least 80%, at least 85%, at least 90%, at least 95%, at least 97% or at least 99% amino acid sequence identity thereof and the second fusion protein comprises the amino acid sequence SEQ ID NO: 73 or SEQ ID NO: 76 or an amino acid sequence having at least 70%, at least 75%, at least 80%, at least 85%, at least 90%, at least 95%, at least 97% or at least 99% amino acid sequence identity thereof.

In an alternative embodiment, the first fusion protein consists of the amino acid sequence SEQ ID NO: 72 or an amino acid sequence having at least 70%, at least 75%, at least 80%, at least 85%, at least 90%, at least 95%, at least 97% or at least 99% amino acid sequence identity thereof.

In an alternative embodiment, the second fusion protein consists of the amino acid sequence SEQ ID NO: 73 or SEQ ID NO: 76 or an amino acid sequence having at least 70%, at least 75%, at least 80%, at least 85%, at least 90%, at least 95%, at least 97% or at least 99% amino acid sequence identity thereof.

In an alternative embodiment, the first fusion protein consists of the amino acid sequence SEQ ID NO: 72 or an amino acid sequence having at least 70%, at least 75%, at least 80%, at least 85%, at least 90%, at least 95%, at least 97% or at least 99% amino acid sequence identity thereof and the second fusion protein consists of the amino acid sequence SEQ ID NO: 73 or SEQ ID NO: 76 or an amino acid sequence having at least 70%, at least 75%, at least 80%, at least 85%, at least 90%, at least 95%, at least 97% or at least 99% amino acid sequence identity thereof.

### Embodiments Wherein the Antigen is the S Protein of SARS-CoV-2.

Examples of fusion proteins targeting the S protein of SARS-CoV-2 among all possible combinations of VHH, linker and naJAZ and noJAZ domains with or without terminal tags are listed in the Table 9 and nine protein fusion pairs among all possible combinations (36) are exemplified below. The S protein of SARS-CoV-2 is an homotrimer and it is possible to bind VHH to the same epitope of two neighbouring monomers at reach of the two fusion proteins.

It is notably exemplified the first fusion proteins VHH704-naJAZ, VHH714-naJAZ and VHH723-naJAZ and the second fusion proteins VHH725-noJAZ, VHH727-noJAZ and VHH724-noJAZ suitable to be used in a system for detecting S protein, preferably S protein of SARS-CoV-2.

VHH704-naJAZ, VHH714-naJAZ, VHH723-naJAZ comprise amino acids MA at their N-terminal end, respectively amino acid sequence SEQ ID NO: 78 of VHH P_S12, SEQ ID NO: 79 of VHH P_H08 or SEQ ID NO: 80 of VHH P_S11, a linker having the amino acid sequence SEQ ID NO: 102, a first fragment of a luciferase having the amino acid sequence SEQ ID NO: 1 (corresponding to amino acids 3-85 of the JAZ luciferase having SEQ ID NO: 4), amino acids LE followed by a histidine tag of SEQ ID NO: 60. VHH725-noJAZ, VHH727-noJAZ, VHH705-noJAZ, VHH724-noJAZ comprise amino acids MA at their N-terminal end, respectively amino acid sequence SEQ ID NO: 79 of VHH P_H08, SEQ ID NO: 79 of VHH P_H08, SEQ ID NO: 78 of VHH P_S12, SEQ ID NO: 78 of VHH P_S12, a linker having the amino acid sequence SEQ ID NO: 102, a second fragment of a luciferase having the amino acid sequence SEQ ID NO: 2 (corresponding to amino acids 86-171 of the JAZ luciferase having SEQ ID NO: 4) for VHH727-noJAZ and VHH724-noJAZ or SEQ ID NO: 114 (corresponding to amino acids 86-171 of the JAZ570 luciferase having SEQ ID NO: 12) for VHH725-noJAZ and VHH705-noJA), amino acids LE followed by a histidine tag of SEQ ID NO: 60.

TABLE 9

| | SEQ | |
|---|---|---|
| | ID | |
| Fusion name | NO | Amino acid sequence |
| VHH704-naJAZ | 90 | MAEVQLQASGGGLVEAGGSLRLSCTTSGLTFSSVTMGWFRQAPGKEREFVAAIRWKFGNLGYADSVKGR |
| (P_S12) | | FTVSRDNAKNTVYLQMNSLKPEDTAVYYCAAARVGEIIAVLISPSNYAYWGQGTQVTVSSAAAGEMETSQ |
| | | NPGEEKPQASPEGRPESETSCLVTTTDNQISTEQGFTLEDFVGDWRQTAGRNLDQVLEQGGVSSLFQNL |
| | | GVSVTPIQRIVKSGENGLKIDIHVIPYEGLSGDQMGQIEKIFKVVYPVLEHHHHHH |
| VHH725- | 91 | MAEVQLQASGGGLVQPGGSLRLSCAASGSFFSISAMGWYRQAPGKQRELVADITSGGSTNYADSVKGRF |
| noJAZ570 | | TISRDNAKNTVYLQMNSLKPEDTAVYYCHVQVGVHPIGYDVWGQGTQVTVSSAAAGEMETSQNPGEEKP |
| (P_H08) | | QASPEGRPESETSCLVTTTDNQISTEQGDDHHFKVILHYGTLVIDGVTPNMIDYFGRPYEGIAVFDGKKITVT |
| | | GTLENGNKIIDERLINPDGSLLFRVTINGVTGERLSERILALEHHHHHH |
| VHH714-naJAZ | 92 | MAEVQLQASGGGLVQPGGSLRLSCAASGSFFSISAMGWYRQAPGKQRELVADITSGGSTNYADSVKGRF |
| (P_H08) | | TISRDNAKNTVYLQMNSLKPEDTAVYYCHVQVGVHPIGYDVWGQGTQVTVSAAAGEMETSQNPGEEKPQ |
| | | ASPEGRPESETSCLVTTTDNQISTEQGFTLEDFVGDWRQTAGRNLDQVLEQGGVSSLFQNLGVSVTPIQRI |
| | | VKSGENGLKIDIHVIPYEGLSGDQMGQIEKIFKVVYPVLEHHHHHH |
| VHH727- | 93 | MAEVQLQASGGGLVQPGGSLRLSCAASGSFFSISAMGWYRQAPGKQRELVADITSGGSTNYADSVKGRF |
| noJAZ570 | | TISRDNAKNTVYLQMNSLKPEDTAVYYCHVQVGVHPIGYDVWGQGTQVTVSAAAGEMETSQNPGEEKPQ |
| (P_H08) | | ASPEGRPESETSCLVTTTDNQISTEQGDDHHFKVILHYGTLVIDGVTPNMIDYFGRPFEGIAVFDGKKITVTG |
| | | TLENGNKIIDERLINPDGSLLFRVTINGVTGERLSERILALEHHHHHH |
| VHH723-naJAZ | 94 | MAQVQLVESGGGLVQAGDSLRLSCAVSGRTFSSLIMGWFRQAPGKEREFVARITYSGGSTHYADSVKGR |
| (P_S11) | | FTISRDNAKNTVYLQMNSLKPEDTAVYYCAADTRGFSWSSSGGYDYWGQGTQVTVASEPKTPKPQPAAA |
| | | GEMETSQNPGEEKPQASPEGRPESETSCLVTTTDNQISTEQGFTLEDFVGDWRQTAGRNLDQVLEQGGV |
| | | SSLFQNLGVSVTPIQRIVKSGENGLKIDIHVIPYEGLSGDQMGQIEKIFKVVYPVLEHHHHHH |
| VHH705- | 95 | MAEVQLQASGGGLVEAGGSLRLSCTTSGLTFSSVTMGWFRQAPGKEREFVAAIRWKFGNLGYADSVKGR |
| noJAZ570 | | FTVSRDNAKNTVYLQMNSLKPEDTAVYYCAAARVGEIIAVLISPSNYAYWGQGTQVTVSSAAAGEMETSQ |
| (P_S12) | | NPGEEKPQASPEGRPESETSCLVTTTDNQISTEQGDDHHFKVILHYGTLVIDGVTPNMIDYFGRPYEGIAVF |
| | | DGKKITVTGTLENGNKIIDERLINPDGSLLFRVTINGVTGERLSERILALEHHHHHI |
| VHH724-noJAZ | 96 | MAEVQLQASGGGLVEAGGSLRLSCTTSGLTFSSVTMGWFRQAPGKEREFVAAIRWKFGNLGYADSVKGR |
| (P_S12) | | FTVSRDNAKNTVYLQMNSLKPEDTAVYYCAAARVGEIIAVLISPSNYAYWGQGTQVTVSSAAAGEMETSQ |
| | | NPGEEKPQASPEGRPESETSCLVTTTDNQISTEQGDDHHFKVILHYGTLVIDGVTPNMIDYFGRPFEGIAVF |
| | | DGKKITVTGTLENGNKIIDERLINPDGSLLFRVTINGVTGERLSERILALEHHHHHH |
| VHH704-naJAZ | 97 | EVQLQASGGGLVEAGGSLRLSCTTSGLTFSSVTMGWFRQAPGKEREFVAAIRWKFGNLGYADSVKGRFT |
| without N and | | VSRDNAKNTVYLQMNSLKPEDTAVYYCAAARVGEIIAVLISPSNYAYWGQGTQVTVSSAAAGEMETSQNP |
| C terminal | | GEEKPQASPEGRPESETSCLVTTTDNQISTEQGFTLEDFVGDWRQTAGRNLDQVLEQGGVSSLFQNLGVS |
| sequences | | VTPIQRIVKSGENGLKIDIHVIIPYEGLSGDQMGQIEKIFKVVYPV |
| VHH725- | 98 | EVQLQASGGGLVQPGGSLRLSCAASGSFFSISAMGWYRQAPGKQRELVADITSGGSTNYADSVKGRFTIS |
| noJAZ570 | | RDNAKNTVYLQMNSLKPEDTAVYYCHVQVGVHPIGYDVWGQGTQVTVSSAAAGEMETSQNPGEEKPQA |
| without N and | | SPEGRPESETSCLVTTTDNQISTEQGDDHHFKVILHYGTLVIDGVTPNMIDYFGRPYEGIAVFDGKKITVTGT |
| C terminal | | LENGNKIIDERLINPDGSLLFRVTINGVTGERLSERILA |
| sequences | | |
| VHH714-naJAZ | 99 | EVQLQASGGGLVQPGGSLRLSCAASGSFFSISAMGWYRQAPGKQRELVADITSGGSTNYADSVKGRFTIS |
| without N and | | RDNAKNTVYLQMNSLKPEDTAVYYCHVQVGVHPIGYDVWGQGTQVTVSAAAGEMETSQNPGEEKPQAS |
| C terminal | | PEGRPESETSCLVTTTDNQISTEQGFTLEDFVGDWRQTAGRNLDQVLEQGGVSSLFQNLGVSVTPIQRIVK |
| sequences | | SGENGLKIDIHVIIPYEGLSGDQMGQIEKIFKVVYPV |
| VHH727-noJAZ | 100 | EVQLQASGGGLVQPGGSLRLSCAASGSFFSISAMGWYRQAPGKQRELVADITSGGSTNYADSVKGRFTIS |
| without N and | | RDNAKNTVYLQMNSLKPEDTAVYYCHVQVGVHPIGYDVWGQGTQVTVSAAAGEMETSQNPGEEKPQAS |
| C terminal | | PEGRPESETSCLVTTTDNQISTEQGDDHHFKVILHYGTLVIDGVTPNMIDYFGRPFEGIAVFDGKKITVTGTL |
| sequences | | ENGNKIIDERLINPDGSLLFRVTINGVTGERLSERILA |
| VHH723-naJAZ | 101 | QVQLVESGGGLVQAGDSLRLSCAVSGRTFSSLIMGWFRQAPGKEREFVARITYSGGSTHYADSVKGRFTI |
| without N and | | SRDNAKNTVYLQMNSLKPEDTAVYYCAADTRGFSWSSSGGYDYWGQGTQVTVASEPKTPKPQPAAAGE |
| C terminal | | METSQNPGEEKPQASPEGRPESETSCLVTTTDNQISTEQGFTLEDFVGDWRQTAGRNLDQVLEQGGVSS |
| sequences | | LFQNLGVSVTPIQRIVKSGENGLKIDIHVIIPYEGLSGDQMGQIEKIFKVVYPV |
| VHH724- | 115 | EVQLQASGGGLVEAGGSLRLSCTTSGLTFSSVTMGWFRQAPGKEREFVAAIRWKFGNLGYADSVKGRFT |
| noJAZ570 | | VSRDNAKNTVYLQMNSLKPEDTAVYYCAAARVGEIIAVLISPSNYAYWGQGTQVTVSSAAAGEMETSQNP |
| without N and | | GEEKPQASPEGRPESETSCLVTTTDNQISTEQGDDHHFKVILHYGTLVIDGVTPNMIDYFGRPFEGIAVFDG |
| C terminal | | KKITVTGTLENGNKIIDERLINPDGSLLFRVTINGVTGERLSERILA |
| sequences | | |
| VHH705- | 116 | EVQLQASGGGLVEAGGSLRLSCTTSGLTFSSVTMGWFRQAPGKEREFVAAIRWKFGNLGYADSVKGRFT |
| noJAZ570 | | VSRDNAKNTVYLQMNSLKPEDTAVYYCAAARVGEIIAVLISPSNYAYWGQGTQVTVSSAAAGEMETSQNP |
| without N and | | GEEKPQASPEGRPESETSCLVTTTDNQISTEQGDDHHFKVILHYGTLVIDGVTPNMIDYFGRPYEGIAVFDG |
| C terminal | | KKITVTGTLENGNKIIDERLINPDGSLLFRVTINGVTGERLSERILA |
| sequences | | |
| VHH687-JAZ | 117 | MAEVQLQASGGGLVEAGGSLRLSCTTSGLTFSSVTMGWFRQAPGKEREFVAAIRWKFGNLGYADSVKGR |
| (P_S12) | | FTVSRDNAKNTVYLQMNSLKPEDTAVYYCAAARVGEIIAVLISPSNYAYWGQGTQVTVSSAAAVFTLEDFVG |
| | | DWRQTAGRNLDQVLEQGGVSSLFQNLGVSVTPIQRIVKSGENGLKIDIHVIIPYEGLSGDQMGQIEKIFKVVY |
| | | PVDDHHFKVILHYGTLVIDGVTPNMIDYFGRPYEGIAVFDGKKITVTGTLENGNKIIDERLINPDGSLLFRVTIN |
| | | GVTGERLCERILALEHHHHHH |
| VHH716-SPB37 | 118 | MAEVQLVESGGGLVEPGGSLRLSCAASGFTWDYYDIGWFRQAPGKEREGVACISSSGSSTNYGDSVKGR |
| (P_S11) | | FTISRDNAKKTVYLQMNSLKPEDTAVYYCAADIVDYGLESASCMWIDRGYWGQGTQVTVSSAAAVFTLEDF |
| | | VGDWRQTAGRNLDQVLEQGGVSSLFQNLGVSVTPIQRIVKSGENGLKIDIHVIIPYEGLSGDQMGQIEKIFKV |
| | | VYPVDDHHFKVILHYGTLVIDGVTPNMIDYFGRPYEGIAVFDGKKITVTGTLENGNKIIDERLINPDGSLLFRVT |
| | | INGVTGERLCERILALEHHHHHH |

In an embodiment, the first fusion protein comprises the amino acid sequence selected from the group consisting of SEQ ID NO: 97, SEQ ID NO: 99, SEQ ID NO: 101 or an amino acid sequence having at least 70%, at least 75%, at least 80%, at least 85%, at least 90%, at least 95%, at least 97% or at least 99% amino acid sequence identity thereof.

In an embodiment, the second fusion protein comprises the amino acid sequence selected from the group consisting of SEQ ID NO: 98, SEQ ID NO: 100, SEQ ID NO: 115 or SEQ ID NO: 116 or an amino acid sequence having at least 70%, at least 75%, at least 80%, at least 85%, at least 90%, at least 95%, at least 97% or at least 99% amino acid sequence identity thereof.

In an embodiment, the first fusion protein comprises the amino acid sequence selected from the group consisting of SEQ ID NO: 97, SEQ ID NO: 99, SEQ ID NO: 101 or an amino acid sequence having at least 70%, at least 75%, at least 80%, at least 85%, at least 90%, at least 95%, at least 97% or at least 99% amino acid sequence identity thereof and the second fusion protein comprises the amino acid sequence selected from the group consisting of SEQ ID NO: 98, SEQ ID NO: 100, SEQ ID NO: 115 or SEQ ID NO: 116 or an amino acid sequence having at least 70%, at least 75%, at least 80%, at least 85%, at least 90%, at least 95%, at least 97% or at least 99% amino acid sequence identity thereof.

In an embodiment, the first fusion protein comprises the amino acid sequence SEQ ID NO: 97 or an amino acid sequence having at least 70%, at least 75%, at least 80%, at least 85%, at least 90%, at least 95%, at least 97% or at least 99% amino acid sequence identity thereof and the second fusion protein comprises the amino acid sequence SEQ ID NO: 98 or an amino acid sequence having at least 70%, at least 75%, at least 80%, at least 85%, at least 90%, at least 95%, at least 97% or at least 99% amino acid sequence identity thereof.

In an embodiment, the first fusion protein comprises the amino acid sequence selected from the group consisting of SEQ ID NO: 90, SEQ ID NO: 92, SEQ ID NO: 94 or an amino acid sequence having at least 70%, at least 75%, at least 80%, at least 85%, at least 90%, at least 95%, at least 97% or at least 99% amino acid sequence identity thereof.

In an embodiment, the second fusion protein comprises the amino acid sequence selected from the group consisting of SEQ ID NO: 91, SEQ ID NO: 93, SEQ ID NO: 95, SEQ ID NO: 96 or an amino acid sequence having at least 70%, at least 75%, at least 80%, at least 85%, at least 90%, at least 95%, at least 97% or at least 99% amino acid sequence identity thereof.

In an embodiment, the first fusion protein comprises the amino acid sequence selected from the group consisting of SEQ ID NO: 90, SEQ ID NO: 92, SEQ ID NO: 94 or an amino acid sequence having at least 70%, at least 75%, at least 80%, at least 85%, at least 90%, at least 95%, at least 97% or at least 99% amino acid sequence identity thereof and the second fusion protein comprises the amino acid sequence selected from the group consisting of SEQ ID NO: 91, SEQ ID NO: 93, SEQ ID NO: 95, SEQ ID NO: 96 or an amino acid sequence having at least 70%, at least 75%, at least 80%, at least 85%, at least 90%, at least 95%, at least 97% or at least 99% amino acid sequence identity thereof.

In an embodiment, the first fusion protein comprises the amino acid sequence SEQ ID NO: 90 or an amino acid sequence having at least 70%, at least 75%, at least 80%, at least 85%, at least 90%, at least 95%, at least 97% or at least 99% amino acid sequence identity thereof and the second fusion protein comprises the amino acid sequence SEQ ID NO: 91 or an amino acid sequence having at least 70%, at least 75%, at least 80%, at least 85%, at least 90%, at least 95%, at least 97% or at least 99% amino acid sequence identity thereof.

In an embodiment, the first fusion protein consists of the amino acid sequence selected from the group consisting of SEQ ID NO: 90, SEQ ID NO: 92, SEQ ID NO: 94 or an amino acid sequence having at least 70%, at least 75%, at least 80%, at least 85%, at least 90%, at least 95%, at least 97% or at least 99% amino acid sequence identity thereof.

In an embodiment, the second fusion protein consists of the amino acid sequence selected from the group consisting of SEQ ID NO: 91, SEQ ID NO: 93, SEQ ID NO: 95, SEQ ID NO: 96 or an amino acid sequence having at least 70%, at least 75%, at least 80%, at least 85%, at least 90%, at least 95%, at least 97% or at least 99% amino acid sequence identity thereof.

In an embodiment, the first fusion protein consists of the amino acid sequence selected from the group consisting of SEQ ID NO: 90, SEQ ID NO: 92, SEQ ID NO: 94 or an amino acid sequence having at least 70%, at least 75%, at least 80%, at least 85%, at least 90%, at least 95%, at least 97% or at least 99% amino acid sequence identity thereof and the second fusion protein consists of the amino acid sequence selected from the group consisting of SEQ ID NO: 91, SEQ ID NO: 93, SEQ ID NO: 95, SEQ ID NO: 96 or an amino acid sequence having at least 70%, at least 75%, at least 80%, at least 85%, at least 90%, at least 95%, at least 97% or at least 99% amino acid sequence identity thereof.

In an embodiment, the first fusion protein consists of the amino acid sequence SEQ ID NO: 90 or an amino acid sequence having at least 70%, at least 75%, at least 80%, at least 85%, at least 90%, at least 95%, at least 97% or at least 99% amino acid sequence identity thereof and the second fusion protein consists of the amino acid sequence SEQ ID NO: 91 or an amino acid sequence having at least 70%, at least 75%, at least 80%, at least 85%, at least 90%, at least 95%, at least 97% or at least 99% amino acid sequence identity thereof.

The present invention also encompasses other combinations. For example, VHH725-naJAZ570, VHH727-naJAZ, VHH705-naJAZ570 or VHH724-naJAZ as first fusion proteins and VHH704-noJAZ, VHH714-noJAZ or VHH723noJAZ as second fusion protein may an alternative combination to VHH704-naJAZ, VHH714-naJAZ or VHH723naJAZ as first fusion proteins and VHH725-noJAZ570, VHH727-noJAZ, VHH705-noJAZ570, VHH724-noJAZ as second fusion proteins.

### Embodiments Wherein the Antigen is the P24 of HIV

P24 is a component of the HIV capsid. The detection of P24 in blood sample is currently used as first test of HIV infection completed with the detection of IgG specific of HIV protein components. Examples of fusion proteins targeting the protein P24 among all possible combinations of anti-P24 VHH, linker and first or second luciferase fragments which are described below and listed in the Table 10 below.

It is notably exemplified the first fusion proteins VHH2XV6_B-linker23-naJAZ, VHH2XV6_B-linker45-naJAZ, VHH59H1-linker23-naJAZ or VHH59H1-linker45-naJAZ and the second fusion protein VHH59H1-linker23-noJAZ VHH59H1-linker45-noJAZ, VHH2XV6_B-linker23-noJAZ or VHH2XV6_B-linker45-noJAZ, suitable to be used in a system for detecting P24.

VHH2XV6_B-linker23-naJAZ (SEQ ID NO: 159) comprises the amino acid sequence SEQ ID NO: 157 of VHH2XV6_B (this VHH sequence includes the heterologous sequence MA), a linker having the amino acid sequence SEQ ID NO: 152, a first fragment of a luciferase having the amino acid sequence SEQ ID NO: 158, amino acids LE followed by a histidine tag of SEQ ID NO: 60.

VHH2XV6_B-linker45-naJAZ (SEQ ID NO: 160) comprises the amino acid sequence SEQ ID NO: 157 of VHH2XV6_B (this VHH sequence includes the heterologous sequence MA), a linker having the amino acid sequence SEQ ID NO: 141, a first fragment of a luciferase having the amino acid sequence SEQ ID NO: 158, amino acids LE followed by a histidine tag of SEQ ID NO: 60.

VHH59H1-linker23-noJAZ (SEQ ID NO: 161) comprises the amino acid sequence SEQ ID NO: 156 of VHH59H1 (this VHH sequence includes the heterologous sequence MA), a linker having the amino acid sequence SEQ ID NO: 152, a second fragment of a luciferase having the amino acid sequence SEQ ID NO: 114, amino acids LE followed by a histidine tag of SEQ ID NO: 60.

VHH59H1-linker45-noJAZ (SEQ ID NO: 162) comprises the amino acid sequence SEQ ID NO: 156 of VHH59H1 (this VHH sequence includes the heterologous sequence MA), a linker having the amino acid sequence SEQ ID NO: 141, a second fragment of a luciferase having the amino acid sequence SEQ ID NO: 114, amino acids LE followed by a histidine tag of SEQ ID NO: 60.

VHH2XV6_B-linker23-noJAZ (SEQ ID NO: 172) comprises the amino acid sequence SEQ ID NO: 157 of VHH2XV6_B (this VHH sequence includes the heterologous sequence MA), a linker having the amino acid sequence SEQ ID NO: 152, a second fragment of a luciferase having the amino acid sequence SEQ ID NO: 114, amino acids LE followed by a histidine tag of SEQ ID NO: 60.

VHH2XV6_B-linker45-noJAZ (SEQ ID NO: 173) comprises the amino acid sequence SEQ ID NO: 157 of VHH2XV6_B (this VHH sequence includes the heterologous sequence MA), a linker having the amino acid sequence SEQ ID NO: 141, a second fragment of a luciferase having the amino acid sequence SEQ ID NO: 114, amino acids LE followed by a histidine tag of SEQ ID NO: 60.

VHH59H1-linker23-naJAZ (SEQ ID NO: 174) comprises the amino acid sequence SEQ ID NO: 156 of VHH59H1 (this VHH sequence includes the heterologous sequence MA), a linker having the amino acid sequence SEQ ID NO: 152, a first fragment of a luciferase having the amino acid sequence SEQ ID NO: 158, amino acids LE followed by a histidine tag of SEQ ID NO: 60.

VHH59H1-linker45-naJAZ (SEQ ID NO: 175) comprises the amino acid sequence SEQ ID NO: 156 of VHH59H1 (this VHH sequence includes the heterologous sequence MA), a linker having the amino acid sequence SEQ ID NO: 141, a first fragment of a luciferase having the amino acid sequence SEQ ID NO: 158, amino acids LE followed by a histidine tag of SEQ ID NO: 60.

TABLE 10

| | SEQ | |
|---|---|---|
| Fusion | ID | |
| name | NO | Amino acid sequence |
| VHH2XV6 | 159 | MADVQLKESGGGLVQAGGSLRLSCAASGSISRFNAMGWWRQAPGKEREFVARIVKGFDPVLADSVKGRFTISIDSAE |
| B-linker23- | | NTLALQMNRLKPEDTAVYYCFAALDTAYWGQGTQVTVSSAAAGEMETSQNPGEEKPQASPEGFTLEDFVGDWRQTA |
| naJAZ | | GRNLDQVLEQGGVSSLFQNLGVSVTPIQRIVKSGENGLKIDIHVIIPYEGLLGDQMGQIEKIFKVVYPVLEHHHHHH |
| VHH2XV6 | 160 | MADVQLKESGGGLVQAGGSLRLSCAASGSISRFNAMGWWRQAPGKEREFVARIVKGFDPVLADSVKGRFTISIDSAE |
| B-linker45- | | NTLALQMNRLKPEDTAVYYCFAALDTAYWGQGTQVTVSSAAAGEMETSQNPGEEKPQASPEGRPESETSCLVTTTD |
| naJAZ | | NQISTEQGFTLEDFVGDWRQTAGRNLDQVLEQGGVSSLFQNLGVSVTPIQRIVKSGENGLKIDIHVIIPYEGLLGDQMG |
| | | QIEKIFKVVYPVLEHHHHHH |
| VHH59H1- | 161 | MAQVQLVESGGGLVQAGGSLRLSCAASGSFFMSNVMAWYRQAPGKARELIAAIRGGDMSTVYDDSVKGRFTITRDDD |
| linker23- | | KNILYLQMNDLKPEDTAMYYCKASGSSWGQGTQVTVSSAAAGEMETSQNPGEEKPQASPEGDDHHFKVILHYGTLVI |
| noJAZ | | DGVTPNMIDYFGRPYEGIAVFDGKKITVTGTLENGNKIIDERLINPDGSLLFRVTINGVTGERLSERILALEHHHHHH |
| VHH59H1- | 162 | MAQVQLVESGGGLVQAGGSLRLSCAASGSFFMSNVMAWYRQAPGKARELIAAIRGGDMSTVYDDSVKGRFTITRDDD |
| linker45- | | KNILYLQMNDLKPEDTAMYYCKASGSSWGQGTQVTVSSAAAGEMETSQNPGEEKPQASPEGRPESETSCLVTTTDN |
| noJAZ | | QISTEQGDDHHFKVILHYGTLVIDGVTPNMIDYFGRPYEGIAVFDGKKITVTGTLENGNKIIDERLINPDGSLLFRVTIN |
| | | GVTGERLSERILALEHHHHHH |
| VHH2XV6 | 172 | MADVQLKESGGGLVQAGGSLRLSCAASGSISRFNAMGWWRQAPGKEREFVARIVKGFDPVLADSVKGRFTISIDSAE |
| B-linker23- | | NTLALQMNRLKPEDTAVYYCFAALDTAYWGQGTQVTVSSAAAGEMETSQNPGEEKPQASPEGDDHHFKVILHYGTLV |
| noJAZ | | IDGVTPNMIDYFGRPYEGIAVFDGKKITVTGTLENGNKIIDERLINPDGSLLFRVTINGVTGERLSERILALEHHHHHH |
| VHH2XV6 | 173 | MADVQLKESGGGLVQAGGSLRLSCAASGSISRFNAMGWWRQAPGKEREFVARIVKGFDPVLADSVKGRFTISIDSAE |
| B-linker45- | | NTLALQMNRLKPEDTAVYYCFAALDTAYWGQGTQVTVSSAAAGEMETSQNPGEEKPQASPEGRPESETSCLVTTTD |
| noJAZ | | NQISTEQGDDHHFKVILHYGTLVIDGVTPNMIDYFGRPYEGIAVEDGKKITVTGTLENGNKIIDERLINPDGSLLFRVTI |
| | | NGVTGERLSERILALEHHHHHH |
| VHH59H1- | 174 | MAQVQLVESGGGLVQAGGSLRLSCAASGSFFMSNVMAWYRQAPGKARELIAAIRGGDMSTVYDDSVKGRFTITRDDD |
| linker23- | | KNILYLQMNDLKPEDTAMYYCKASGSSWGQGTQVTVSSAAAGEMETSQNPGEEKPQASPEG |
| naJAZ | | FTLEDFVGDWRQTAGRNLDQVLEQGGVSSLFQNLGVSVTPIQRIVKSGENGLKIDIHVIIPYEGLLGDQMGQIEKIFKVV |
| | | YPVLEHHHHHH |
| VHH59H1- | 175 | MAQVQLVESGGGLVQAGGSLRLSCAASGSFFMSNVMAWYRQAPGKARELIAAIRGGDMSTVYDDSVKGRFTITRDDD |
| linker45- | | KNILYLQMNDLKPEDTAMYYCKASGSSWGQGTQVTVSSAAAGEMETSQNPGEEKPQASPEGRPESETSCLVTTTDN |
| naJAZ | | QISTEQGFTLEDFVGDWRQTAGRNLDQVLEQGGVSSLFQNLGVSVTPIQRIVKSGENGLKIDIHVIIPYEGLLGDQMGQI |
| | | EKIFKVVYPVLEHHHHHH |

In an embodiment, the first fusion protein comprises or consists of the amino acid sequence selected from the group consisting of SEQ ID NO: 159, SEQ ID NO: 160, SEQ ID NO: 174 and SEQ ID NO: 175 or an amino acid sequence having at least 70%, at least 75%, at least 80%, at least 85%, at least 90%, at least 95%, at least 97% or at least 99% amino acid sequence identity thereof.

In an embodiment, the second fusion protein comprises or consists of the amino acid sequence selected from the group consisting of SEQ ID NO: 161, SEQ ID NO: 162, SEQ ID NO: 172 and SEQ ID NO: 173 or an amino acid sequence having at least 70%, at least 75%, at least 80%, at least 85%, at least 90%, at least 95%, at least 97% or at least 99% amino acid sequence identity thereof.

In a preferred embodiment, the first fusion protein comprises or consists of the amino acid sequence SEQ ID NO: 159 or an amino acid sequence having at least 70%, at least 75%, at least 80%, at least 85%, at least 90%, at least 95%, at least 97% or at least 99% amino acid sequence identity thereof, more preferably SEQ ID NO: 160 or an amino acid sequence having at least 70%, at least 75%, at least 80%, at least 85%, at least 90%, at least 95%, at least 97% or at least 99% amino acid sequence identity thereof and the second fusion protein comprises or consists of the amino acid sequence SEQ ID NO: 161 or an amino acid sequence having at least 70%, at least 75%, at least 80%, at least 85%, at least 90%, at least 95%, at least 97% or at least 99% amino acid sequence identity thereof.

### B. Polynucleotides, Vectors and Cells

The present invention also relates to a polynucleotide encoding the fusion protein of the invention. Typically, a first polynucleotide may encode the first fusion protein as defined above and/or a second polynucleotide may encode the second fusion protein as defined above.

In an embodiment, the first polynucleotide encodes the first fusion protein comprising the amino acid sequence selected from the group consisting of SEQ ID NO: 66, SEQ ID NO: 68, SEQ ID NO: 72, SEQ ID NO:74, SEQ ID NO: 90, SEQ ID NO:92, SEQ ID NO: 94, SEQ ID NO: 97, SEQ ID NO: 99, SEQ ID NO: 101, SEQ ID NO: 159, SEQ ID NO: 160, SEQ ID NO: 174 and SEQ ID NO: 175 or an amino acid sequence having at least 70%, at least 75%, at least 80%, at least 85%, at least 90%, at least 95%, at least 97% or at least 99% amino acid sequence identity thereof. Preferably, the first polynucleotide encodes the first fusion protein comprising the amino acid sequence selected from the group consisting of SEQ ID NO: 66, SEQ ID NO:72, SEQ ID NO: 90, SEQ ID NO:92, SEQ ID NO:94 or an amino acid sequence having at least 70%, at least 75%, at least 80%, at least 85%, at least 90%, at least 95%, at least 97% or at least 99% amino acid sequence identity thereof. More preferably, the first polynucleotide encodes the first fusion protein consisting in the amino acid sequence selected from the group consisting of SEQ ID NO: 66, SEQ ID NO: 90 or an amino acid sequence having at least 70%, at least 75%, at least 80%, at least 85%, at least 90%, at least 95%, at least 97% or at least 99% amino acid sequence identity thereof.

In an embodiment, the second polynucleotide encodes the second fusion protein comprising the amino acid sequence selected from the group consisting of SEQ ID NO: 67, SEQ ID NO: 69, SEQ ID NO: 70, SEQ ID NO:71, SEQ ID NO: 73, SEQ ID NO:75, SEQ ID NO: 76, SEQ ID NO:77, SEQ ID NO: 91, SEQ ID NO:93, SEQ ID NO: 95, SEQ ID NO: 96, SEQ ID NO: 98, SEQ ID NO: 100, SEQ ID NO: 115, SEQ ID NO: 116, SEQ ID NO: 161, SEQ ID NO:162, SEQ ID NO:172 and SEQ ID NO: 173, or an amino acid sequence having at least 70%, at least 75%, at least 80%, at least 85%, at least 90%, at least 95%, at least 97% or at least 99% amino acid sequence identity thereof. Preferably, the second polynucleotide encodes the second fusion protein comprising the amino acid sequence selected from the group consisting of SEQ ID NO: 67, SEQ ID NO:70, SEQ ID NO:73, SEQ ID NO:76, SEQ ID NO: 91, SEQ ID NO:93 and SEQ ID NO:95, SEQ ID NO: 96, or an amino acid sequence having at least 70%, at least 75%, at least 80%, at least 85%, at least 90%, at least 95%, at least 97% or at least 99% amino acid sequence identity thereof. More preferably, the second polynucleotide encodes the second fusion protein consisting in the amino acid sequence selected from the group consisting of SEQ ID NO: 67, SEQ ID NO:70, SEQ ID NO: 91, SEQ ID NO:93 and SEQ ID NO:95, SEQ ID NO: 96, or an amino acid sequence having at least 70%, at least 75%, at least 80%, at least 85%, at least 90%, at least 95%, at least 97% or at least 99% amino acid sequence identity thereof. More preferably, the second polynucleotide encodes the second fusion protein consisting in the amino acid sequence selected from the group consisting of SEQ ID NO: 67, SEQ ID NO: 91 or an amino acid sequence having at least 70%, at least 75%, at least 80%, at least 85%, at least 90%, at least 95%, at least 97% or at least 99% amino acid sequence identity thereof.

Suitably the polynucleotides of the invention are recombinant. Recombinant means that the polynucleotide is the product of at least one of cloning, restriction or ligation steps, or other procedures that result in a polynucleotide that is distinct from a polynucleotide found in nature.

Advantageously, the polynucleotide may be codon-optimized for expression of the fusion protein (first and/or second fusion protein) in a host cell.

The present invention also relates to a vector comprising the polynucleotide of the invention.

As used herein, vector (or plasmid) refers to discrete elements that are used to introduce heterologous DNA into cells for either expression or replication thereof. Selection and use of such vehicles are well-known to those of skill in the art. An expression vector includes vectors capable of expressing DNAs that are operatively linked with regulatory sequences, such as promoters, that are capable of effecting expression of such DNA fragments. Thus, an expression vector refers to a recombinant DNA construct, such as a plasmid, a phage, recombinant virus or other vector that, upon introduction into an appropriate host cell, results in expression of the cloned DNA. Appropriate expression vectors are well known to those of skill in the art.

A recombinant vector is a vector comprising a recombinant polynucleotide.

Advantageously, the vector comprises the polynucleotide operably linked to a promoter. As used herein, operatively linked refers to the functional relationship of DNA with regulatory and effector sequences of nucleotides, such as promoters, enhancers, transcriptional and translational stop sites, and other signal sequences.

For example, operative linkage of DNA to a promoter refers to the physical and functional relationship between the DNA and the promoter such that the transcription of such DNA is initiated from the promoter by an RNA polymerase that specifically recognizes, binds to and transcribes the DNA.

As used herein, a promoter refers to a segment of DNA that controls transcription of the DNA to which it is operatively linked.

The polynucleotide or the vector of the invention may be into a cell, typically a prokaryote or eukaryote cell. The vector may be conservative in the cytoplasm or the polynucleotide could be integrated in the genome using lentiviral vector or genome edition (i.e. CRISPR-Cas9 but not limited to).

Therefore, the present invention also relates to a cell comprising the polynucleotide of the invention or the expression vector of the invention.

### C. System

The present invention also relates to a system for detecting an antigen comprising the first fusion protein as defined above and the second fusion protein as defined above. Advantageously, luminescence is emitted in the presence of a substrate when both the first fusion protein and the second fusion protein bind to said antigen.

A method to determine if the first fusion protein and the second fusion protein are suitable to emit luminescence when they are both bound to their antigen could be designed by a person skilled in the art based on the present specification, the examples below and its general knowledge.

For example, 90 μL of a premix comprising the first fusion protein at 1 μg/mL+the second fusion protein at 0.2 μg/mL+8-(2,3-difluorobenzyl)-2-((5-methylfuran-2-yl)methyl)-6-phenylimidazo[1,2-a]pyrazin-3(7H)-one (Q-108) at 25 μM+DTT 5 mM+Tween 20 0.05% in PBS is loaded in a clear polystyrene tube. The background of bioluminescence signal (wide light intensity peak centred at 460 nm measured as relative light intensity unit per second, RLU/s) is recorded along a 5 s-kinetics with sampling every 0.5 s. The background drift (RLU/s²) and noise amplitude (RLU/s) are computed from these 10 points 5 s. About 10 μL of sample comprising 1 μM of the antigen is added and mixed to the 90 μL of reacting solution. The kinetic activity is recorded for 10 to 60 s with a 0.5 s integration time (RLU/s and RLU/s²). The background noise is extrapolated from the noise drift and the delay between the noise recording and the kinetics points. If the slope of the kinetic rate (RLU/s²) is more than twice the drift or if the corrected slope is flat and the light emission (RLU/s) is 5 times greater than the background noise, the first and the second fusion proteins are considered suitable for use in a system according to the invention for detecting the antigen. It is considered the measurement system as semi-quantitative if the sensitivity of the measurement of the antigen concentration is above 100 nM (risk of underestimating the concentration with a slow binding kinetic) and quantitative below 100 nM (equivalent to 4.5 μg/mL of antigen, 45 μg/mL before 1/10^{th} dilution). Higher is the sdAb pair affinity for the antigen, lower is the sensitivity threshold, better is the accuracy.

Advantageously, the first and the second fusion proteins are two separate elements of the system according to the invention. They are not covalently linked. They are only assembled together when they are both bound to the antigen and form a complex with the antigen.

In an embodiment, the system for detecting an antigen comprises:
a first fusion protein comprising:
a N-terminal domain which comprises a first variable domain of a camelid heavy-chain antibody (VHH) which is directed against a first epitope of said antigen
   and
a C-terminal domain which comprises a first fragment of a luciferase:
   wherein the first fragment has:
the amino acid sequence as set forth in SEQ ID NO: 1 or
an amino acid sequence having at least 70% amino acid sequence identity to the amino acid sequence as set forth in SEQ ID NO: 1,
   and
a second fusion protein comprising:
a N-terminal domain which comprises a second VHH which is directed against a second epitope of said antigen
   and
a C-terminal domain which comprises a second fragment of a luciferase:
   wherein the second fragment has:
the amino acid sequence as set forth in SEQ ID NO: 2 or
an amino acid sequence having at least 70% amino acid sequence identity to the amino acid sequence as set forth in SEQ ID NO: 2,
   luminescence being emitted in the presence of a substrate when both the first fusion protein and the second fusion protein bind to said antigen.

In an embodiment, the antigen to be detected by the system of the invention is a nucleoprotein (N protein), preferably N protein of SARS-CoV-2.

In this embodiment, the first and/or the second VHH may have:
an amino acid sequence selected from the group consisting of SEQ ID NO: 20 to 29 or
an amino acid sequence that is at least 80%, at least 85%, at least 90%, at least 95%, at least 97% or at least 99% identical to an amino acid sequence selected from the group consisting of SEQ ID NO: 20 to 29.

In the embodiment where the antigen is N protein, the first sdAb, preferably VHH, of the first fusion protein may be a VHH directed against the CTD of N protein and the second sdAb, preferably VHH of the second fusion protein may be a VHH directed against the NTD of the N protein or conversely.

In an embodiment, the first VHH may be the VHH having the amino acid sequence SEQ ID NO: 23 and the second VHH may be the VHH having the amino acid sequence SEQ ID NO: 25 or conversely.

In an embodiment, the first fusion protein comprises the amino acid sequence selected from the group consisting of SEQ ID NO: 68, SEQ ID NO:74 or an amino acid sequence having at least 70%, at least 75%, at least 80%, at least 85%, at least 90%, at least 95%, at least 97% or at least 99% amino acid sequence identity thereof and the second fusion protein comprises the amino acid sequence selected from the group consisting of SEQ ID NO: 69, SEQ ID NO:71, SEQ ID NO:75 and SEQ ID NO:77 or an amino acid sequence having at least 70%, at least 75%, at least 80%, at least 85%, at least 90%, at least 95%, at least 97% or at least 99% amino acid sequence identity thereof.

In an embodiment, the first fusion protein consists of the amino acid sequence selected from the group consisting of SEQ ID NO: 66, SEQ ID NO:72, or an amino acid sequence having at least 70%, at least 75%, at least 80%, at least 85%, at least 90%, at least 95%, at least 97% or at least 99% amino acid sequence identity thereof and the second fusion protein consists of the amino acid sequence selected from the group consisting of SEQ ID NO: 67, SEQ ID NO:70, SEQ ID NO:73 and SEQ ID NO:76, or an amino acid sequence having at least 70%, at least 75%, at least 80%, at least 85%, at least 90%, at least 95%, at least 97% or at least 99% amino acid sequence identity thereof.

In an embodiment, the antigen to be detected by the system of the invention is a spike protein (S protein), preferably S protein of SARS-CoV-2.

In this embodiment, the first and/or the second VHH may have the amino acid sequence selected from the group consisting of SEQ ID NO: 78, SEQ ID NO:79 SEQ ID NO: 80, SEQ ID NO: 126, SEQ ID NO: 127, SEQ ID NO: 128, SEQ ID NO: 129 and SEQ ID NO: 130 or an amino acid sequence having at least 70%, at least 75%, at least 80%, at least 85%, at least 90%, at least 95%, at least 97% or at least 99% amino acid sequence identity thereof.

In this embodiment, the first fusion protein may comprise the amino acid sequence selected from the group consisting of SEQ ID NO: 97, SEQ ID NO:99 and SEQ ID NO:101 or an amino acid sequence having at least 70%, at least 75%, at least 80%, at least 85%, at least 90%, at least 95%, at least 97% or at least 99% amino acid sequence identity thereof and the second fusion protein may comprise the amino acid sequence selected from the group consisting of SEQ ID NO: 96, SEQ ID NO:98 and SEQ ID NO:100 or an amino acid sequence having at least 70%, at least 75%, at least 80%, at least 85%, at least 90%, at least 95%, at least 97% or at least 99% amino acid sequence identity thereof.

In an embodiment, the first fusion protein consists of the amino acid sequence selected from the group consisting of SEQ ID NO: 90, SEQ ID NO:92 and SEQ ID NO:94 or an amino acid sequence having at least 70%, at least 75%, at least 80%, at least 85%, at least 90%, at least 95%, at least 97% or at least 99% amino acid sequence identity thereof and the second fusion protein consists of the amino acid sequence selected from the group consisting of SEQ ID NO: 91, SEQ ID NO:93, SEQ ID NO:95 and SEQ ID NO:96 or an amino acid sequence having at least 70%, at least 75%, at least 80%, at least 85%, at least 90%, at least 95%, at least 97% or at least 99% amino acid sequence identity thereof.

In the embodiment wherein the antigen is P24, the first sdAb, preferably VHH, of the first fusion protein and the second sdAb, preferably VHH, of the second fusion protein are directed against P24.

In an embodiment, the first VHH may comprises or consists of the amino acid sequence SEQ ID NO: 156 and the second VHH may comprises or consists of the amino acid sequence SEQ ID NO: 157 or conversely.

In an embodiment, the first fusion protein comprises the amino acid sequence selected from the group consisting of SEQ ID NO: 159, SEQ ID NO: 160 and SEQ ID NO: 174 and SEQ ID NO: 175 or an amino acid sequence having at least 70%, at least 75%, at least 80%, at least 85%, at least 90%, at least 95%, at least 97% or at least 99% amino acid sequence identity thereof and the second fusion protein comprises the amino acid sequence selected from the group consisting of SEQ ID NO: 161, SEQ ID NO: 162, SEQ ID NO: 172 and SEQ ID NO: 173 or an amino acid sequence having at least 70%, at least 75%, at least 80%, at least 85%, at least 90%, at least 95%, at least 97% or at least 99% amino acid sequence identity thereof.

In an embodiment, the first fusion protein consists of the amino acid sequence selected from the group consisting of SEQ ID NO: 159, SEQ ID NO: 160, SEQ ID NO: 174 and SEQ ID NO: 175 or an amino acid sequence having at least 70%, at least 75%, at least 80%, at least 85%, at least 90%, at least 95%, at least 97% or at least 99% amino acid sequence identity thereof and the second fusion protein consists of the amino acid sequence selected from the group consisting of SEQ ID NO: 161, SEQ ID NO: 162, SEQ ID NO: 172 and SEQ ID NO: 173 or an amino acid sequence having at least 70%, at least 75%, at least 80%, at least 85%, at least 90%, at least 95%, at least 97% or at least 99% amino acid sequence identity thereof.

### D. Complex

Another subject matter of the invention is a complex comprising:
a first fusion protein comprising:
   a N-terminal domain which comprises a first single domain antibody which is directed against a first epitope of an antigen
      and
a C-terminal domain which comprises a first fragment of a luciferase, wherein the first fragment has:
the amino acid sequence as set forth in SEQ ID NO: 1 or
an amino acid sequence having at least 70% %, at least 75%, at least 80%, at least 85%, at least 90%, at least 95%, at least 97% or at least 99% amino acid sequence identity to the amino acid sequence as set forth in SEQ ID NO: 1,
   wherein the first fusion protein has no luciferase activity
a second fusion protein comprising:
   a N-terminal domain which comprises a second single domain antibody which is directed against a second epitope of an antigen
      and
a C-terminal domain which comprises a second fragment of a luciferase, wherein the second fragment has:
the amino acid sequence as set forth in SEQ ID NO: 2 or
an amino acid sequence having at least 70% %, at least 75%, at least 80%, at least 85%, at least 90%, at least 95%, at least 97% or at least 99% amino acid sequence identity to the amino acid sequence as set forth in SEQ ID NO: 2,
   wherein the second fusion protein has no luciferase activity
   and
   the antigen;
   the first and the second fusion proteins being both bound to the antigen.

An embodiment of the invention relates to a complex comprising:
a first fusion protein comprising:
   a N-terminal domain which comprises a first variable domain of a camelid heavy-chain antibody (VHH) which is directed against a first epitope of said antigen and
   a C-terminal domain which comprises a first fragment of a luciferase:
wherein the first fragment has:
   the amino acid sequence as set forth in SEQ ID NO: 1 or
   an amino acid sequence having at least 70%, at least 75%, at least 80%, at least 85%, at least 90%, at least 95%, at least 97% or at least 99% amino acid sequence identity to the amino acid sequence as set forth in SEQ ID NO: 1,
a second fusion protein comprising:
   a N-terminal domain which comprises a second VHH which is directed against a second epitope of said antigen
      and
a C-terminal domain which comprises a second fragment of a luciferase:
wherein the second fragment has:
   the amino acid sequence as set forth in SEQ ID NO: 2 or
   an amino acid sequence having at least 70%, at least 75%, at least 80%, at least 85%, at least 90%, at least 95%, at least 97% or at least 99% amino acid sequence identity to the amino acid sequence as set forth in SEQ ID NO: 2,
      and
      the antigen; the first and the second fusion proteins being both bound to the antigen.

Typically, the complex according to the invention comprises:
a first fusion protein as defined above,
a second protein as defined above
   and the antigen; the first fusion protein and the second fusion protein being both bound to the antigen.

Typically, the complex according to the invention has a luciferase activity. The luciferase activity is recovered by the antigen-driven reassembly of luciferase fragments carried by the two complementary fusion proteins. The fusion protein pair and the substrate may be premixed for measuring the background drift then the sample containing the antigen is added for measuring the light emission increase.

### E. Kit

A subject matter of the present invention is also a kit comprising:
the system of the invention and
a substrate for the luciferase.

Typically, the kit comprises the first fusion protein according to the invention, the second fusion protein according to the invention and a substrate for the luciferase.

Coelenterazine is the natural substrate for the shrimp Oplophorus luciferase but improvement in signals may be obtained with furimazine and even more improvement with deacylated-hikarazine.

Consequently, the substrate may be selected from the group consisting of coelenterazine, furimazine and deacylated-hikarazine or derivatives thereof.

Derivatives of deacylated-hikarazine are disclosed in the patent application WO2018/197727 A1. Such derivatives of deacylated-hikarazine provide a better bioluminescence signals in term of intensity, signal-to-noise ratio and/or duration than other luciferins.

Consequently, the substrate may be selected in the group consisting in:
8-benzyl-2-((5-methylfuran-2-yl)methyl)-6-phenylimidazo[1,2-a]pyrazin-3(7H)-one
8-benzyl-2-((5-ethylfuran-2-yl)methyl)-6-phenylimidazo[1,2-a]pyrazin-3(7H)-one
8-benzyl-2-((4,5-dimethylfuran-2-yl)methyl)-6-phenylimidazo[1,2-a]pyrazin-3(7H)-one
8-benzyl-6-(2-fluorophenyl)-2-(furan-2-ylmethyl)imidazo[1,2-a]pyrazin-3(7H)-one
8-benzyl-2-(3-methylbenzyl)-6-phenylimidazo[1,2-a]pyrazin-3(7H)-one
8-benzyl-2-(3-methoxybenzyl)-6-phenylimidazo[1,2-a]pyrazin-3(7H)-one
2,8-dibenzyl-6-(2-fluorophenyl)imidazo[1,2-a]pyrazin-3(7H)-one
8-benzyl-6-(2,6-difluorophenyl)-2-(furan-2-ylmethyl)imidazo[1,2-a]pyrazin-3(7H)-one
8-benzyl-6-phenyl-2-((5-(trifluoromethyl)furan-2-yl)methyl)imidazo[1,2-a]pyrazin-3(7H)-one
2,8-dibenzyl-6-(2,6-difluorophenyl)imidazo[1,2-a]pyrazin-3(7H)-one
8-benzyl-6-(2-fluorophenyl)-2-((5-methylfuran-2-yl)methyl)imidazo[1,2-a]pyrazin-3(7H)-one
8-benzyl-2-((5-cyclopropylfuran-2-yl)methyl)-6-phenylimidazo[1,2-a]pyrazin-3(7H)-one
8-benzyl-2-(3-fluorobenzyl)-6-phenylimidazo[1,2-a]pyrazin-3(7H)-one
8-benzyl-2-((5-ethylfuran-2-yl)methyl)-6-(2-fluorophenyl)imidazo[1,2-a]pyrazin-3(7H)-one
8-benzyl-6-(3-fluorophenyl)-2-((5-methylfuran-2-yl)methyl)imidazo[1,2-a]pyrazin-3(7H)-one
8-benzyl-2-(2-fluorobenzyl)-6-phenylimidazo[1,2-a]pyrazin-3(7H)-one
8-benzyl-2-((5-ethylthiophen-2-yl)methyl)-6-phenylimidazo[1,2-a]pyrazin-3(7H)-one
8-benzyl-2-((4,5-dimethylfuran-2-yl)methyl)-6-(2-fluorophenyl)imidazo[1,2-a]pyrazin-3(7H)-one
2-benzyl-8-(2-fluorobenzyl)-6-phenylimidazo[1,2-a]pyrazin-3(7H)-one
2-benzyl-8-(3-fluorobenzyl)-6-phenylimidazo[1,2-a]pyrazin-3(7H)-one
8-(3-fluorobenzyl)-2-(furan-2-ylmethyl)-6-phenylimidazo[1,2-a]pyrazin-3(7H)-one
8-(2-fluorobenzyl)-2-(3-methylbenzyl)-6-phenylimidazo[1,2-a]pyrazin-3(7H)-one
8-(2-fluorobenzyl)-2-(3-methoxybenzyl)-6-phenylimidazo[1,2-a]pyrazin-3(7H)-one
8-(2-fluorobenzyl)-2-(furan-2-ylmethyl)-6-phenylimidazo[1,2-a]pyrazin-3(7H)-one
8-(2-fluorobenzyl)-2-((5-methylfuran-2-yl)methyl)-6-phenylimidazo[1,2-a]pyrazin-3(7H)-one
8-(3-fluorobenzyl)-2-(3-methoxybenzyl)-6-phenylimidazo[1,2-a]pyrazin-3(7H)-one
8-(3-fluorobenzyl)-2-(3-methylbenzyl)-6-phenylimidazo[1,2-a]pyrazin-3(7H)-one
2-((5-ethylfuran-2-yl)methyl)-8-(3-fluorobenzyl)-6-phenylimidazo[1,2-a]pyrazin-3(7H)-one
8-(2-chlorobenzyl)-2-((5-methylfuran-2-yl)methyl)-6-phenylimidazo[1,2-a]pyrazin-3(7H)-one
8-(3-fluorobenzyl)-2-((5-methylfuran-2-yl)methyl)-6-phenylimidazo[1,2-a]pyrazin-3(7H)-one
2-((5-ethylfuran-2-yl)methyl)-8-(2-fluorobenzyl)-6-phenylimidazo[1,2-a]pyrazin-3(7H)-one
8-(3-fluorobenzyl)-6-(2-fluorophenyl)-2-((5-methylfuran-2-yl)methyl)imidazo[1,2-a]pyrazin-3(7H)-one
8-(2,3-difluorobenzyl)-2-(furan-2-ylmethyl)-6-phenylimidazo[1,2-a]pyrazin-3(7H)-one
8-(2,3-difluorobenzyl)-2-((5-methylfuran-2-yl)methyl)-6-phenylimidazo[1,2-a]pyrazin-3(7H)-one
2-benzyl-8-(2,3-difluorobenzyl)-6-phenylimidazo[1,2-a]pyrazin-3(7H)-one
8-(2,6-Difluorobenzyl)-2-((5-methylfuran-2-yl)methyl)-6-phenylimidazo[1,2-a]pyrazin-3(7H)-one
8-(2,3-Difluorobenzyl)-2-((4,5-dimethylfuran-2-yl)methyl)-6-phenylimidazo[1,2-a]pyrazin-3(7H)-one
8-(2,3-Difluorobenzyl)-2-((5-ethylfuran-2-yl)methyl)-6-phenylimidazo[1,2-a]pyrazin-3(7H)-one
8-(2,6-Difluorobenzyl)-2-((5-ethylfuran-2-yl)methyl)-6-phenylimidazo[1,2-a]pyrazin-3(7H)-one
2-((4,5-Dimethylfuran-2-yl)methyl)-8-(2-fluorobenzyl)-6-phenylimidazo[1,2-a]pyrazin-3(7H)-one
2-((4,5-Dimethylfuran-2-yl)methyl)-8-(3-fluorobenzyl)-6-phenylimidazo[1,2-a]pyrazin-3(7H)-one
8-(2,3-difluorobenzyl)-2-((4-ethyl-5-methylfuran-2-yl)methyl)-6-phenylimidazo[1,2-a]pyrazin-3(7H)-one
8-(2,3-difluorobenzyl)-2-((5-ethyl-4-methylfuran-2-yl)methyl)-6-phenylimidazo[1,2-a]pyrazin-3(7H)-one and 8-benzyl-2-(furan-2-ylmethyl)-6-(3-hydroxyphenyl)imidazo[1,2-a]pyrazin-3(7H)-one.

These substrates are respectively disclosed in WO2018/197727 A1 with the following names Q3, Q12, Q16, Q21, Q14, Q18, Q20, Q27, Q28, Q29, Q34, Q36, Q41, Q51, Q54, Q56, Q58, Q61, Q72, Q73, Q81, Q82, Q83, Q84, Q85, Q101, Q100, Q99, Q98, Q97, Q96, Q105, Q107, Q108, Q117, Q121, Q124, Q127, 0129, Q131, Q132, Q135, Q143 and Q149.

In a preferred embodiment, the substrate is 8-(2,3-difluorobenzyl)-2-((5-methylfuran-2-yl)methyl)-6-phenylimidazo[1,2-a]pyrazin-3(7H)-one (Q-108 as disclosed in Table 1 page 129 of WO2018/197727 A1).

In an embodiment, the concentration of the substrate is between 5 μM and 200 μM, preferably between 10 μM and 175 μM.

The first fusion protein, the second fusion protein and the substrate may be packaged separately or packaged together in the same premix. In a particular embodiment said premix comprises the first and second fusion proteins, the substrate, DTT 5 mM and Tween 200.1% in a buffer (e.g. phosphate buffer saline (PBS)).

The kit may also comprise reagents for the detection of luciferase activity, a negative and/or positive control sample, a tube and/or either swab, an inoculation loop, a split pin, a stick, a paper or a plastic stripe.

The fusion protein VHH-anti N-FcIg1 having the amino acid sequence SEQ ID NO: 121 comprises a signal peptide having the amino acid sequence SEQ ID NO: 123, the VHH anti-N protein G9-1 having the amino acid sequence SEQ ID NO: 23, a linker having the amino acid sequence SEQ ID NO: 124, the Fc of an immunoglobulin G1 (IgG1) having the amino acid sequence SEQ ID NO: 125 and a HisTag. The VHH-anti N-FcIg1 may be used as positive control notably to calibrate a method for detecting or quantifying a N protein of SARS-CoV-2, in particular a serological method or a method according to the invention.

The fusion protein VHH-anti S-FcIg1 having the amino acid sequence SEQ ID NO: 122 comprises a signal peptide having the amino acid sequence SEQ ID NO: 123, the VHH anti-S protein P S12 having the amino acid sequence SEQ ID NO: 78, a linker having the amino acid sequence SEQ ID NO: 124, the Fc of an immunoglobulin G1 (IgG1) having the amino acid sequence SEQ ID NO: 125 and a HisTag. The VHH-anti S-FcIg1 may be used as positive control notably to calibrate a method for detecting or quantifying a S protein of SARS-CoV-2, in particular a serological method or a method according to the invention.

In an embodiment, the ratio first fusion protein/second fusion protein is between 10/1 and 1/1, preferably between 7/1 and 2/1, more preferably about 5/1. Such ratios enable to lower the background noise.

The kit as described above may be used for detecting and/or quantifying the antigen in a biological sample for prognosis, diagnosis and therapy follow-up purposes.

### F. Method

The present invention also relates to the use of the system according to the invention for detecting and/or quantifying the antigen in a sample.

Typically, the invention relates to the use of a first fusion protein as defined above and a second fusion protein as defined above for detecting and/or quantifying the antigen in a sample.

A subject matter of the present invention is also a method for detecting the presence of an antigen in a sample comprising the steps of:
(a) contacting the sample with the system as defined above and a substrate of the luciferase,
(b) detecting the luminescence (RLU/s) and eventually measuring the increasing rate of the luminescence (RLU/s²).

The method may enable to detect the antigen in less than a minute.

Typically, in step (a) the first fusion protein as defined above, the second fusion protein as defined above and a substrate for the luciferase as defined above are contacted with the sample.

Since the level of antigen in the sample may be also measured by the mean to the emitted luminescence, the present invention also relates to a method for quantifying the presence of an antigen in a sample comprising the steps of:
(a) contacting the sample with the system as defined above and a substrate of the luciferase,
(b) quantifying the luminescence (RLU/s) and eventually the increasing rate of the luminescence (RLU/s²).

Typically, in step (a) the first fusion protein as defined above, the second fusion protein as defined above and a substrate for the luciferase are contacted with the sample.

The sample may be for example selected from the group consisting of:
human or animal body fluids such as:
   whole blood, serum, plasma, cerebrospinal fluid, sperm, urine, nasopharyngeal smear, oropharyngeal smear, vaginal smear, skin smear, stool, sweat, saliva, tracheal washing and/or bronchial washing.
human, animal, vegetal, bacterial, fungal or parasite cell lysate or tissue extract such as:
   lysate from cells after sonication, pressurization/depressurization (French press, syringe), bead smashing, thawing-freezing cycles, cryofracture, potterization, gun particles, enzymatic or detergent rupture or solubilization of cytoplasmic membrane, nuclear membrane or organelle membrane, etc. If necessary, the clarification of the lysate can be processed by centrifugation. The step of lysis may be preceded of either tissue washing liquid, tissue smear suspension or blended tissue.
environmental liquid or smear such as:
   water river, puddle, pond, lake, sea, ocean, fountain, tank or recipient of water or any beverage or liquid, sewage, washing effluent, cooling systems or smear of solid matter in sewage, garbage, environment, building or houses, or smear of any surface of any material exposed or not.
food and drug such as:
   solid raw or cooked food, raw, natural or industrialized food ingredient or drug are blended, resuspended and eventually clarified by centrifugation, liquid drug dilution.

Preferably, the sample is a biological sample selected among serum, saliva, rhino-pharyngeal or nasal swab wash, urine and/or feces smear.

The volume of the sample may be from 0.1 μl to 5 ml, preferably, from 1 μl to 100 μl more preferably from 5 μl to 50 μl.

For example, in tube reader of bioluminescence, the volume of the sample may be f 0.1 μL to 5 mL (maximal volume of a standard polystyrene crystal tube), typically 5 to 50 μl completed by a buffer (e.g. phosphate buffer saline (PBS)) for a total volume of 100 μl that can be extended to 5 mL. In plate reader of bioluminescence, the volume of the sample may be for example 0.1 μL to 50 μL, typically 5 to 50 μL completed by the complementary fusion protein pair and substrate in buffer (e.g. phosphate buffer saline (PBS)) for a total volume of 100 μL that can be extended to 3 mL in 96 deep well plate with flat bottom. Assay works with transparent plate (clear polystyrene) but preferred plates are white with flat bottom encompassing 96 to 384 wells. For 1536 well plate, the volume of the sample may be for example 0.1 μL to 5 μL, typically 1 to 5 μL completed by the complementary fusion protein pair and substrate in buffer (e.g. phosphate buffer saline (PBS)) for a total volume of 10 μL.

Preferably, the pH of the sample is between 7 and 9.

The substrate may be any substrate as defined above, preferably, 8-(2,3-difluorobenzyl)-2-((5-methylfuran-2-yl)methyl)-6-phenylimidazo[1,2-a]pyrazin-3(7H)-one (the deacetylated hikarazine called Q-108 in WO2018/197727 A1).

The methods may also comprise a step of comparing to the luminescence emitted by a control. The control may be a positive and/or a negative control. The negative control may be a blank control or a sample obtained from a healthy subject i.e. a subject who does not suffer from the disorder which the antigen is indicative. The positive control may a sample comprising a given concentration of the antigen to be assayed or a sample from a subject suffering from the disorder which the antigen is indicative.

In the embodiment wherein the antigen is quantified, the method may comprise a step of comparison with a calibration curve, usually a serial dilution of the antigen.

When detecting the luminescence, the number of photons per second may be counted eventually according to their wavelength.

When the level of antigen in a sample is quantified, the luminescence can be quantified and the light intensity versus antigen concentration may be plotted.

The method of the invention may comprise no coating step and/or no washing step.

The method of the invention may also comprise no incubation step.

The luciferase activity may be recovered by complementation measured versus time using for example a luminometer or a high-light sensitivity camera.

In an embodiment, the ratio: first fusion protein/second fusion protein is between 10/1 and 1/1, preferably between 7/1 and 2/1, more preferably about 5/1. Such ratios enable to lower the background noise.

In an embodiment, the method of the invention is for detecting and/or quantifying an N protein, preferably the N protein of SARS-CoV-2.

In another embodiment, the method of the invention is for detecting and/or quantifying a S protein, preferably the S protein of SARS-CoV-2.

In another embodiment, the method of the invention is for detecting and/or quantifying P24 in a sample.

The invention will be further illustrated by the following figures and examples. However, these examples and figures should not be interpreted in any way as limiting the scope of the present invention.

### FIGURES

FIG. 1 is a scheme structural domain topology of fusions proteins VHH677-naJAZ (A, SEQ ID NO: 66) and VHH690-noJAZ (B, SEQ ID NO: 67) targeting the SARS-CoV-2 Nucleoprotein, and the VHH704-naJAZ (C, SEQ ID NO: 90) and VHH725-noJAZ (D, SEQ ID NO: 91) targeting the SARS-CoV-2 Spike.

FIG. 2 is showing comparative schemes of the reaction for (A) the detection of SARS-CoV-2 Nucleoprotein serologic antibodies detected by an antibody fused to a luciferase using antigens immobilized on plate or tube surface, (B) the detection of SARS-CoV-2 Nucleoprotein using a sandwich of specific antibodies with one (VHH655-SBP37, SEQ ID NO: 120) bound to streptavidin (STRP) adsorbed to plate, tube, stripe or membrane surface and one fused to the luciferase (VHH648-JAZ, SEQ ID NO: 119), (C) the detection of SARS-CoV-2 Spike using a sandwich of specific antibodies with one (VHH716-SBP37, SEQ ID NO: 118) bound to streptavidin (STRP) adsorbed to plate, tube, stripe or membrane surface and one fused to the luciferase (VHH687-JAZ, SEQ ID NO: 117), (D) the detection of the free Nucleoprotein using the premix comprising the VHH677-naJAZ (SEQ ID NO: 66) with the linker spacing the two domains and VHH690-noJAZ (SEQ ID NO: 67) with the linker spacing the two domains and the substrate Q108, (E) the detection of the free or virus borne Spike using the premix comprising the VHH704-naJAZ (SEQ ID NO: 90) with the linker spacing the two domains and VHH725-noJAZ (SEQ ID NO: 91) with the linker spacing the two domains and the substrate Q108.

FIG. 3 The linear dynamic scale (difference of signal between min and max) is plotted versus the percentage of saliva diluted in PBS/Tween 20 0.05%. Detection threshold in PBS/Tween 20 0.05% 10 pM, 0.4 ng/mL up to 10% of saliva.

FIG. 4 shows dilution series of (A,B) Nucleoprotein starting from 10 fM (pg/mL) to 0.1 μM (100 ng/mL) in PBS or (C,D) Spike starting from 10 fM (pg/mL) to 0.1 μM (100 ng/mL) in PBS using the premix comprising the VHH704-naJAZ (SEQ ID NO: 90) and VHH725-noJAZ (SEQ ID NO: 91) and the substrate Q108. The detection threshold by LuLIFlash in PBS is 50 pM. The raw data are shown on A,C, the average and the standard errors are plotted at the bottom (B,D).

FIG. 5 shows LuLIFlash'N and LuLIFlash'S from reference positive and negative samples. Dilution of saliva ( 1/10) in PBS of 48 negative samples validated by RT-qPCR used as reference. The measurement on theses individual's samples were duplicated. 48 wells were loaded with reagent mix and 10% of the same saliva (Same) and 1 μg/mL of purified recombinant Nucleoprotein (A) or Spike (B). 48 wells were loaded with reagent mix and 10% of the saliva from 48 different individuals (negative) and 1 μg/mL of purified recombinant Nucleoprotein or Spike (Different).

FIG. 6 shows LuLIFlash'N using the premix comprising the VHH677-naJAZ (SEQ ID NO: 66) and VHH690-noJAZ (SEQ ID NO: 67) and the substrate Q108 and LuLIFlash'S using the premix comprising the VHH704-naJAZ (SEQ ID NO: 90) and VHH725-noJAZ (SEQ ID NO: 91) and the substrate Q108 for assaying the antigen concentration in positive and negative samples from 96 different individuals for each of the two groups. Dilution of saliva ( 1/10) in PBS of 96 negative and 96 positive samples validated by a standard ELISA assay using a sandwich of antibodies anti-N, one (VHH716-SBP37, SEQ ID NO: 118) bound to streptavidin well-coated, the other one linked to a luciferase (VHH687-JAZ, SEQ ID NO: 117) as described in FIG. 2B and page 9. For the measurement, 96 wells were loaded with reagent mix (90 μL) and 10% of saliva (1 μL) from the negative individuals (A, B, D) and 96 from the positive individuals (A,C,E) for the SARS-CoV-2 Nucleoprotein. The reagent mix is made of VHH677-naJAZ (SEQ ID NO: 66), VHH690-noJAZ (SEQ ID NO: 67), Tween 20, DTT, PBS. VHH677-naJAZ/VHH690-noJAZ is representative of the most preferred pairs for Nucleoprotein assays. Whisker-box plots indicate quartiles Q2 and Q3 and min and max from values acquired versus time (seconds) along reaction kinetics. Medians are splitting the boxes. (A) Experimental values are plotted aside (time=20 min). Differences in SARS-CoV-2 Nucleoprotein levels between samples from negative and positive samples were compared using an unpaired Mann-Whitney U test. P values<0.001 are considered statistically significant. Whisker-boxes are plotted with relative intensity units per second (RLU/s) from negative (B) and positive (C) samples versus time. A positive threshold is figured by a dashed line at 25,000 RLU/s set from negative controls. Whisker-box are plotted of relative intensity unit increasing rate per second square (RLU/s²) from negative (D) and positive (E) samples versus time. A positive threshold is figured by a dashed line at 500 RLU/s² set from negative controls.

FIG. 7 shows LuLIFlash'S from positive and negative samples from 96 different individuals for each of the two groups. Dilution of saliva ( 1/10) in PBS of 96 negative and 96 positive samples validated by a standard ELISA assay using a sandwich of antibodies anti-S one (VHH716-SBP37, SEQ ID NO: 118) bound to streptavidin well-coated, the other one linked to a luciferase (VHH687-JAZ, SEQ ID NO: 117) as described in the FIG. 2C and page 9. For the measurement, 96 wells were loaded with reagent mix (90 μL) and 10% of saliva (10 μL) from the negative individuals (A, B, D) and 96 from the positive individuals (A, C, E) for the SARS-CoV-2 Spike. The reagent mix is made of VHH704-naJAZ (SEQ ID NO: 90), VHH725-noJAZ (SEQ ID NO: 91), Tween 20, DTT, PBS. VHH704-naJAZ/VHH725-noJAZ is representative of the most preferred pairs for Spike assays. Whisker-box plots indicate quartiles Q2 and Q3 and min and max from values acquired versus time (seconds) along reaction kinetics. Medians are splitting the boxes. (A) Experimental values are plotted aside (time=20 min). Differences in SARS-CoV-2 Spike levels between samples from negative and positive samples were compared using an unpaired Mann-Whitney U test. P values<0.001 are considered statistically significant. Whisker-box are plotted with relative intensity units per second (RLU/s) from negative (B) and positive (C) samples versus time. A positive threshold is figured by a dashed line at 25,000 RLU/s set from negative controls. Whisker-box are plotted of relative intensity unit increasing rate per second square (RLU/s²) from negative (D) and positive (E) samples versus time. A positive threshold is figured by a dashed line at 500 RLU/s² set from negative controls.

FIG. 8 is giving an overview of the field protocol. (A) The reactive mix with fusion pairs and substrate in the appropriate buffer is loaded in a tube (10 μL to 5 mL, preferentially 100 μL) and the background signal is recorded. (B) The sample of a saliva is collected in individual mouth (here 10 μL with a plastic loop at the tip of a stick, commercially distributed as a sterile inoculating loop). (C) The loop is loaded in the tube mixing the sample with the reactive. (D) The signal of bioluminescence is recorded versus time for 10 to 60 seconds: samples are positive either if the measurement is greater than the RLU/s threshold (25,000 RLU/s in the FIG. 6 or 7) or if the increasing rate is greater than the RLU/s² threshold (500 RLU/s in the FIG. 6 or 7) while the thresholds have been set from negative sample series. In the absence of samples series and the use of single negative control, thresholds may be set as twice the bioluminescence (RLU/s) or twice the bioluminescence increasing rate (RLU/s²) of negative controls.

FIG. 9 shows the ratio of bioluminescence signal of various combination of anti-P24 VHH-linker with 23 or 45 residues—naJAZ and anti-P24 VHH-linker with 23 or 45 residues—noJAZ with and without P24. The P24 concentration is of 4 microg/mL.

FIG. 10 shows the ratio of bioluminescence signal of various combination of anti-P24 VHH-linker with 23 or 45 residues—naJAZ and anti-P24 VHH-linker with 23 or 45 residues—noJAZ with and without P24 at different concentrations of P24.

### EXAMPLES

### Human Samples

Samples come from several epidemiologic cohorts approved by ethical committees.

### Design and Synthesis of Plasmid Encoding the Anti-Nucleoprotein-Luciferase Tandem (pET23-vhh677-linker-najaz and pET23-vhh690-linker-nojaz)

The two SARS-CoV-2 N binding moieties VHH G9 (SEQ ID NO: 24) and VHH C7.1 (SEQ ID NO: 26) are issued by M13-phage display from a library of variable domains from single heavy chain antibodies (PF Recombinant antibody, Institut Pasteur) of alpacas (farm at Rennemoulin, Yvelines, France) immunized with the antigen. The gene G9 and C7.1 have been amplified from M13 phagemid with the corresponding forward and reverse oligonucleotides using a Q5 DNA polymerase, dNTP mix (New England BioLabs). PCR products were purified by electrophoresis on agarose gel (1%, Macherey Nagel).

JAZ (SEQ ID NO: 4) is an optimized sequence of the catalytic domain of the luciferase from Oplophorus gracilirostris, with mutations Y116F, C166S, Y18R, L48K, W134E, W163E introduced in addition to the 16 that differentiate the KAZ (SEQ ID NO: 3) from the wild type catalytic domain.

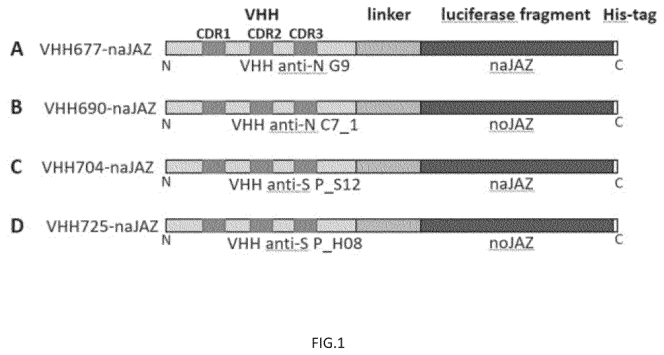

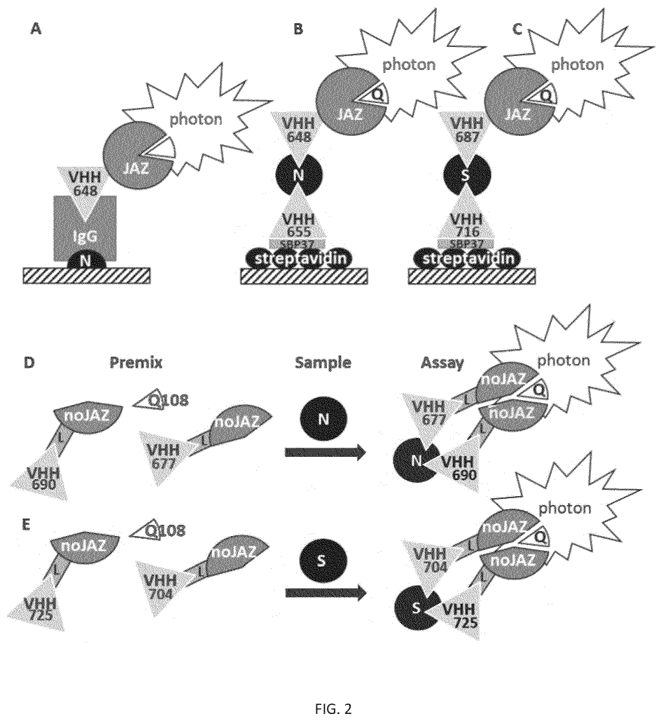

The gene KAZ has been optimized then synthetized by Eurofins (Germany) mutations, carboxy-end (LE), His6-tag (SEQ ID NO: 60) and flanking region corresponding to the pET23 sequence (Novagen). pET23 plasmid has been amplified with the forward and reverse oligonucleotides using a Q5 DNA polymerase, dNTP mix (New England BioLabs). PCR product was purified by electrophoresis on agarose gel (1%, Macherey Nagel). Purified pET23 vector and the synthetic gene were assembled (pET23-kaz) using NEBuilder HiFi assembly master mix (New England BioLabs). The 6 mutations have been introduced in the KAZ gene by PCR. The amino-end (3-85=naJAZ, SEQ ID NO: 1) and carboxy-end (86-171=noJAZ, SEQ ID NO: 2) domains have been assembled in C-terminus of a synthetic oligo-nucleotide encoding a linker spacing the gene of VHH G9 (VHH677-naJAZ) and VHH C7.1 (VHH690-noJAZ) using the Gibson method and then been subcloned in a plasmid pET23. The topology of constructs is detailed in the FIG. 1.

### Expression, Purification and Validation of Fusion Proteins VHH677-naJAZ and VHH690-noJAZ

pET23-VHH677-naJAZ and pET23-VHH690-noJAZ were used separately to transform E. coli BL21 (DE3, New-England Biolabs) to achieve high expression in E. coli. Cells were grown at 16° C. and IPTG (Sigma-Aldrich) was added to induce VHH677-naJAZ or VHH690-noJAZ production. After harvesting the cells by centrifugation (1.5 L), the pellet was resuspended in 50 mM Tris-HCl pH 8.0, 50 mM NaCl with protease inhibitor (Sigma-Aldrich) and lysozyme (0.1 mg/mL, Sigma-Aldrich). Cells were disrupted by freezing-thawing cycle lysis method. DNase I (Sigma-Aldrich) was then added to remove DNA from the sample.

The crude extract was centrifuged 30 min at 1250 g. The supernatant was collected and NaCl (500 mM), Imidazole (20 mM, Sigma-Aldrich) and Triton X-100 (0.1%, Sigma-Aldrich) were added. The cleared lysate was loaded on an equilibrated Hi-Trap 5 mL-column (GE-Healthcare) at 4 mL/min using an AKTA pure chromatography system (GE-Healthcare). The column was washed with 20 volumes of column with a running buffer (50 mM Tris-HCl pH 8.0, NaCl 50 mM, 20 mM imidazole) at 5 mL/min. The VHH677-naJAZ or VHH690-noJAZ were eluted with a gradient of imidazole from 20 mM to 200 mM in 50 mM Tris-HCl pH 8.0, 50 mM NaCl at 5 mL/min and fractions of 1 mL were collected in 96-deepwell plate (GE-Healthcare). The relative concentration of the purified protein was assessed by loading an aliquot (10 μL) on a stain-free SDS gel (4-15% Mini-PROTEAN® TGX Stain-Free™ Protein Gels, Bio-Rad). The gel was activated by UV trans-illumination for 5 min (Bio-Gel Doc XR Imaging System). Tryptophan residues undergo an UV-induced reaction with trihalo compounds and produce a fluorescence signal imaged. The fractions of high concentration were pooled, and loaded on a 1 mL HiTrap Q column (GE-Healthcare) equilibrated in 50 mM Tris-HCl pH 8.0, NaCl 50 mM. The protein was eluted in 50 mM MES pH 6.5, 50 mM NaCl at 1 mL/min at 18° C. using the AKTA pure chromatography system. The fractions of 500 μL were collected in 96-deepwell plate and their concentration were assayed from gels as described above. The fractions of high concentration were pooled. An UV-spectrum (240-300 nm) was acquired for evaluating the concentration of VHH677-naJAZ or VHH690-noJAZ from the solution absorption at 280 nm.

The specific activity of JAZ is about 10¹⁵ acquired photons/second/mg with furimazine in PBS at 23° C. The optimal activity is reached for a substrate (furimazine) concentration from 10 to 30 μM (plateau at about 10 times the K_{M}=2 μM). Beyond 30 μM the dipolar moments of the substrates out of the JAZ (or KAZ as well) catalytic site are quenching the photon emission of the catalyzed substrate in the active site. Quenching efficiency depends on dipolar moment of substrates. Substrate catalysis inactivates stochastically the JAZ (or KAZ as well) and the lifetime of enzyme depends on substrates and catalysis rate substrates (Coutant, Goyard et al. OBC 2019, 17, 3709-3713; Coutant, et al. Chemistry 2020, 26, 948-958; Goyard et al. Allergy 2021, 75, 2952-2956). The split JAZ complementation recovers up to 15% of the uncut JAZ. The split JAZ are still inactivated by reaction product and we still observe inhibition by excess of substrate. The reaction is very sensitive to pH, depending to samples the buffer concentration can be adapted to maintain the reaction between 7.4 and 8.0. Typical the reaction is performed in PBS, buffered by 10 mM of phosphate (pH 7.4), salt keeps most proteins, nucleic acids and complex structure (NaCl 150 mM), detergent avoid unspecific interaction and tube wall absorption (Tween 20 0.05%). The best substrate tested among the 172 furimazine analogs synthesized by Yves Janin's team is the deacetylated-hikarazine-108 or Q108 described in the patent application (EP 3395803, WO2018197727). The optimal substrate concentration of Q108 is in between 13 and 50 μM.

### LuLIFlash'N Protocol

This method called also LuLIFlash'N has been developed for samples collected from rhino-pharyngeal swab extracting solution or saliva from buccal loop but it is compatible also with urine, tear, serum samples or blood drop although concentration of SARS-CoV-2 Nucleoprotein is rather low in these body fluids. It is also compatible with feces smear extracting solution enriched in viral proteins in COVID-19 patients. The following reactive solutions are stored at 4° C.: 1) VHH677-naJAZ 1 mg/mL, DTT 5 mM Tween 20 0.5% in PBS; 2) VHH690-noJAZ 200 μg/mL, DTT 5 mM Tween 20 0.05% in PBS; 3) Q108 5 mM in DMSO/ethanol/HCl; 4) PBS, DTT 5 mM, Tween 20 0.05%.

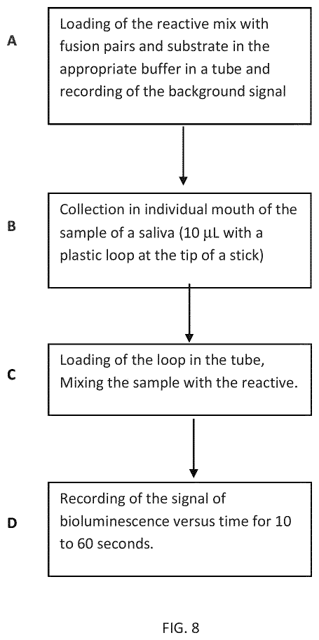

The FIG. 8 is giving an overview of the field protocol. Typically for a single measurement on site, a premix of reaction buffer stable for hours at 4° C. (90 μL: VHH677-naJAZ 1 μg/mL+VHH690-noJAZ 0.2 μg/mL+Q108 25 μM+DTT 5 mM+Tween 20 0.05% in PBS) is loaded in a clear polystyrene tube. The background of bioluminescence signal (wide light intensity peak centred at 460 nm) is recorded along a 5 s-kinetics with sampling every 0.5 s (RLU/s). The background drift (RLU/s²) and noise amplitude (RLU/s) are computed from these 10 points. About 10 μL of sample (the content of a saliva loop) is added and mixed to the 90 μL of reacting solution in the 5 mL polystyrene crystal tube. The kinetic activity is recorded from 10 to 60 s with a 0.5 s integration time. The background noise is extrapolated from the noise drift and the delay between the noise recording and the kinetics points. If the slope of the bioluminescence increasing rate (RLU/s²) is more than twice the drift, the sample is considered positive. If the corrected slope is flat and the bioluminescence (RLU/s) is 2 times greater than the background noise, the sample is considered positive. Calibration is done with a tube containing a known concentration SARS-CoV2 Nucleoprotein.

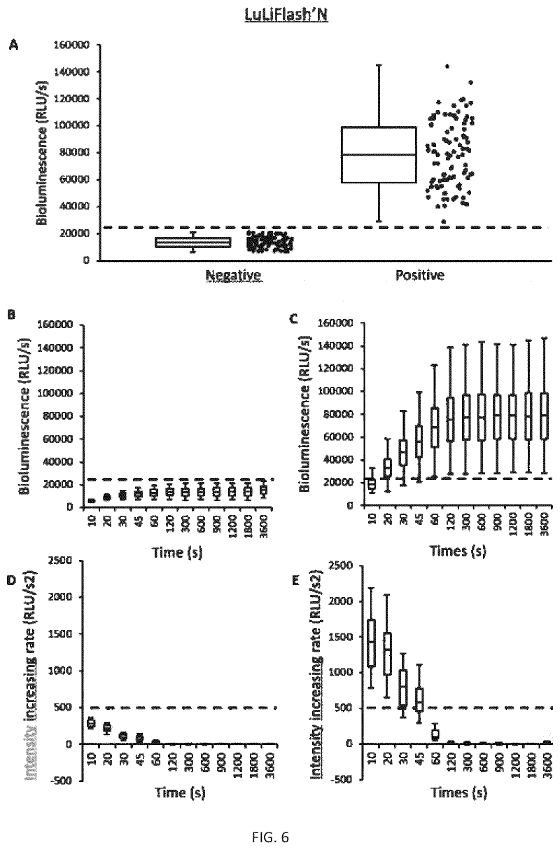

For large number of analysis, a premix of reaction buffer stable for hours at 4° C. (90 μL: VHH677-naJAZ 1 μg/mL+VHH690-noJAZ 0.2 μg/mL+Q108 25 μM+DTT 5 mM+Tween 20 0.05% in PBS) is loaded in 96 or 384 wells of white plates with flat bottom (Fluoronunc C96 or C384 Maxisorp, Nunc). VHH677-naJAZ/VHH690-noJAZ is representative of our best preferred pairs for assaying Nucleoprotein. The background of bioluminescence is recorded along a three points-kinetics with sampling every 0.5 s or read 3 times along the 3 reading the full plate. The background drift and noise amplitude are computed from these 3 points. As shown in FIG. 8 about 10 μL of sample (the content of a saliva loop) is added and mixed to the 90 μL of reacting solution in the tube. The kinetic activity is either recorded for 10 to 60 s with a 0.5 s integration time or read 3 times along the reading the full plate. The background noise is extrapolated from the noise drift and the delay between the noise recording and the kinetics points. If the slope (RLU/s²) is more than twice the drift, the sample is considered positive. If the corrected slope is flat and the bioluminescence (RLU/s) is 2 times greater than the background noise, the sample is considered positive as shown in the FIG. 6. Calibration is done with a tube containing a known concentration SARS-CoV-2 Nucleoprotein. Bioluminescence threshold (RLU/s) and bioluminescence increasing rate threshold (RLU/s²) may be adjusted using negative sample series from characterized healthy donors or negative reference as shown in the FIG. 6.

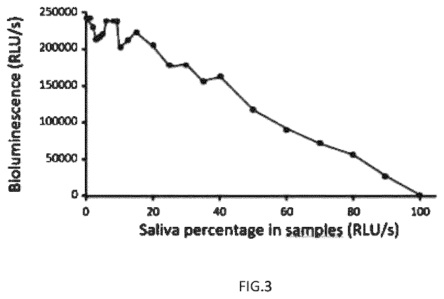

The dynamic range (5-log) and the sensibility (10 μM) is detailed respectively in the FIGS. 3A and B showing the 24 repeats of dilution series in the same 384-well plate. The concentration of saliva affects the signal by raising the background noise and kill the signal at 100% saliva content as shown in the FIG. 3B. A loss of the optimal sensitivity beyond 10% is observed while the dynamic range is already cut by 20%.

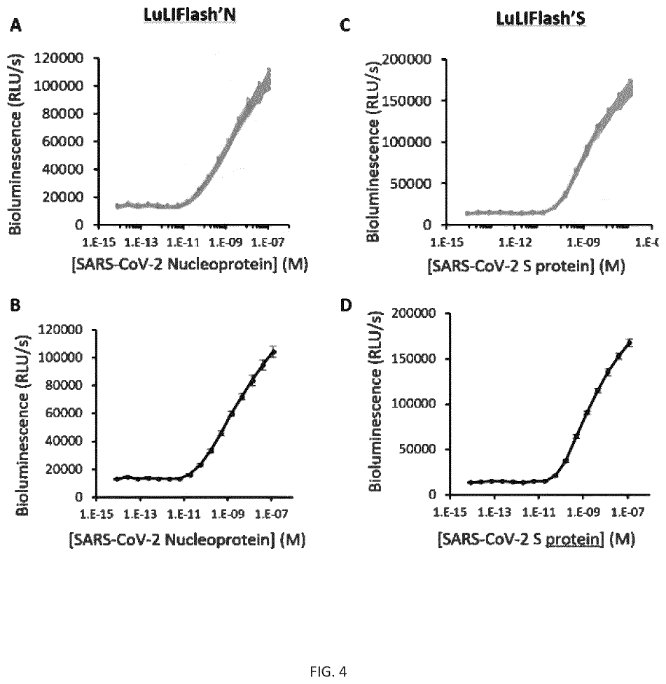

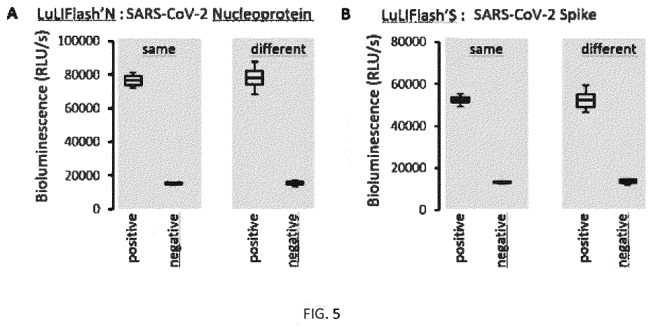

The measurements are reproducible as shown in the FIGS. 4 and 5.

### Sensitivity of the SARS-CoV-2 Nucleoprotein Detection Using the LuLIFlash'N in Different Samples.

This method is also compatible with single blood drop. 1/50^{th} dilution of blood is enough to provide reliable quantitative detection of the Nucleoprotein with LuLIFlash'N. The fingertip is punctured with a device as those used by diabetic patients, 10 μL of blood is collected with a loop or a capillary tube and mixed with 500 μL of a reactive premix. However, the concentration of SARS-CoV-2 viral particle or proteins are rather low in the circulating blood in the infected people while the concentration of specific IgG is rather high competing with the VHH pair used in the assay. Examples of Nucleoprotein assays performed on 96 negative and 96 positive samples are shown in the FIG. 6.

Performance of the LuLIFlash'N with Different Storage Conditions of Reagents

Assays were repeatedly performed using aliquoted Nucleoprotein in PBS solution (1 μg/mL) and reagent solutions VHH677-naJAZ (1 mg/mL), VHH690-noJAZ (1 mg/mL) and Q108 (5.4 mM) at −80° C., −20° C. and +4° C. along 2 months. Conclusions are VHH677-naJAZ, VHH690-noJAZ moderately sensitive to thawing process and they preserve most of their activity at 4° C. for 2 months: 88%, 92 and 94% for storage at +4, −20 and −80° C.

### LuLIFlash's Protocol

A similar method has been also for detecting and assaying SARS-CoV-2 spike also in samples collected from rhino-pharyngeal swab extracting solution or saliva from buccal loop but it is also compatible also with urine, tear, serum samples or blood drop although concentration of SARS-CoV-2 spike is rather low in these body fluids. It is also compatible with feces smear extracting solution enriched in viral proteins in COVID-19 patients. The following reactive solutions are stored at 4° C.: 1) VHH704-naJAZ (SEQ ID NO 93) 1 mg/mL DTT 5 mM Tween 20 0.05% in PBS; 2) VHH725-noJAZ (SEQ ID NO 94) 200 μg/mL DTT 5 mM Tween 20 0.05% in PBS; 3) Q108 5 mM in DMSO/ethano/HCl; 4) PBS, DTT 5 mM, Tween 20 0.05%.

The FIG. 8 is giving an overview of the field protocol. Typically for a single measurement on site, a premix of reaction buffer stable for hours at 4° C. (90 μL: VHH704-naJAZ 1 μg/mL+VHH725-noJAZ 0.2 μg/mL+Q108 25 μM+DTT 5 mM+Tween 20 0.05% in PBS) is loaded in a clear polystyrene tube. The background of bioluminescence signal (wide light intensity peak centred at 460 nm) is recorded along a 5 s-kinetics with sampling every 0.5 s. The background drift and noise amplitude are computed from these 10 points. About 10 μL of sample (the content of a saliva loop) is added and mixed to the 90 μL of reacting solution in the 5 mL polystyrene crystal tube. The kinetic activity is recorded from 10 to 60 s with a 0.5 s integration time (RLU/s). The background noise is extrapolated from the noise drift and the delay between the noise recording and the kinetics points. If the slope of the bioluminescence intensity increasing rate (RLU/s²) is more than twice the drift, the sample is considered positive. If the corrected slope is flat and the bioluminescence (RLU/s) is 2 times greater than the background noise, the sample is considered positive. Calibration is done with a tube containing a known concentration SARS-CoV-2 Spike.

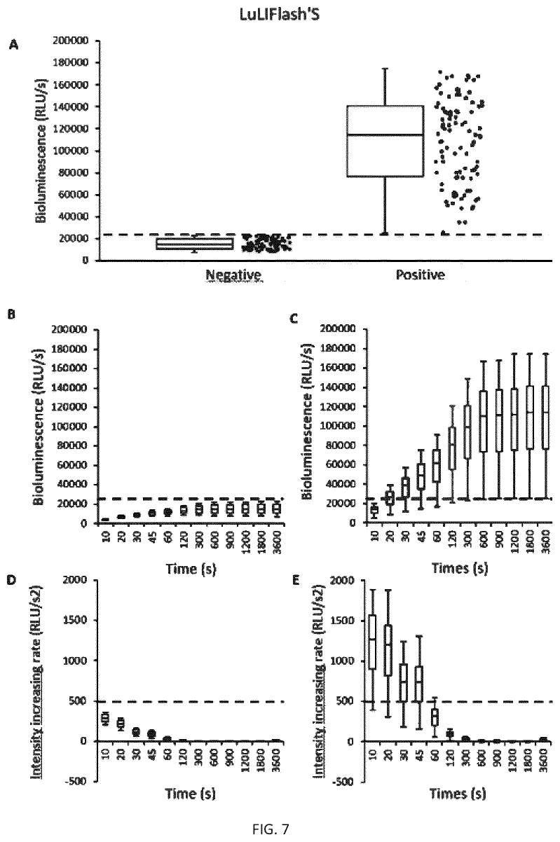

For large number of analysis, a premix of reaction buffer stable for hours at 4° C. (90 μL: VHH704-naJAZ 1 μg/mL+VHH725-noJAZ 0.2 μg/mL+Q108 25 μM+DTT 5 mM+Tween 20 0.05% in PBS) is loaded in 96 or 384 wells of white plates with flat bottom (Fluoronunc C96 or C384 Maxisorp, Nunc). VHH704-naJAZ/VHH725-noJAZ is representative of our best preferred pairs for assaying Spike. The background of bioluminescence is recorded along a three points-kinetics with sampling every 0.5 s or read 3 times along the 3 reading the full plate. The background drift and noise amplitude are computed from these 3 points. As shown in the FIG. 7 about 10 μL of sample (the content of a saliva loop) is added and mixed to the 90 μL of reacting solution in the tube. The kinetic activity is either recorded for 10 to 60 s with a 0.5 s integration time or read 3 times along the reading the full plate. The background noise is extrapolated from the noise drift and the delay between the noise recording and the kinetics points. If the slope of the bioluminescence intensity increasing rate (RLU/s²) is more than twice the drift, the sample is considered positive. If the corrected slope is flat and the bioluminescence (RLU/s) is 2 times greater than the background noise, the sample is considered positive. Calibration is done with a tube containing a known concentration SARS-CoV-2 Spike. Bioluminescence threshold (RLU/s) and bioluminescence increasing rate threshold (RLU/s²) may be adjusted using negative sample series from characterized healthy donors or negative reference as shown in the FIG. 7.

The dynamic range (5-log) and the sensibility (10 μM) is detailed respectively in the FIGS. 4A and B. The concentration of saliva affects the signal by raising the background noise and kill the signal at 100% saliva content. A loss of the optimal sensitivity beyond 10% is observed while the dynamic range is already cut by 20%. This assay is also detecting the spike proteins carried at the surface of SARS-CoV-2 capsid, and consequently detect the viral particles.

### Sensitivity of the SARS-CoV-2 Spike Detection Using the LuLiFlash'S in Different Samples.

This method is also compatible with single blood drop. 1/50th dilution of blood is enough to provide reliable quantitative detection of the SARS-CoV-2 Spike with LuLIFlash'S. The fingertip is punctured with a device as those used by diabetic patients, 10 μL of blood is collected with a loop or a capillary tube and mixed with 500 μL of a reactive premix. However, the concentration of SARS-CoV-2 viral particle or proteins are rather low in the circulating blood in the infected people while the concentration of specific IgG could be high competing with the VHH pair used in the assay.

Examples of Spike assays performed on 96 negative and 96 positive saliva samples are shown in the FIG. 7.

Performance of the LuLIFlash'S with Different Storage Conditions of Reagents

Assays were repeatedly performed using aliquoted spike in PBS solution (1 μg/mL) and reagent solutions VHH704-naJAZ (1 mg/mL), VHH725-noJAZ (1 mg/mL) and Q108 (5.4 mM) at −80° C., −20° C. and +4° C. along 6 months. Conclusions are VHH704-naJAZ, VHH725-noJAZ moderately sensitive to thawing process and they preserve most of their activity at 4° C. for 2 months: 80%, 88% and 92% for storage at +4, −20 and −80° C. respectively.

### LuLIFlash'P24 Protocol

An instant bioassay has been developed with the method LuLiFlash for the detection of one of the reference markers of HIV infection, the protein P24 from HIV capsid in body fluids.

The structure of both VHH have been co-crystallized with P24. The respective epitope of the two VHH have no intersection and far away from each other at least for avoiding any steric hindrance of the bound VHH.

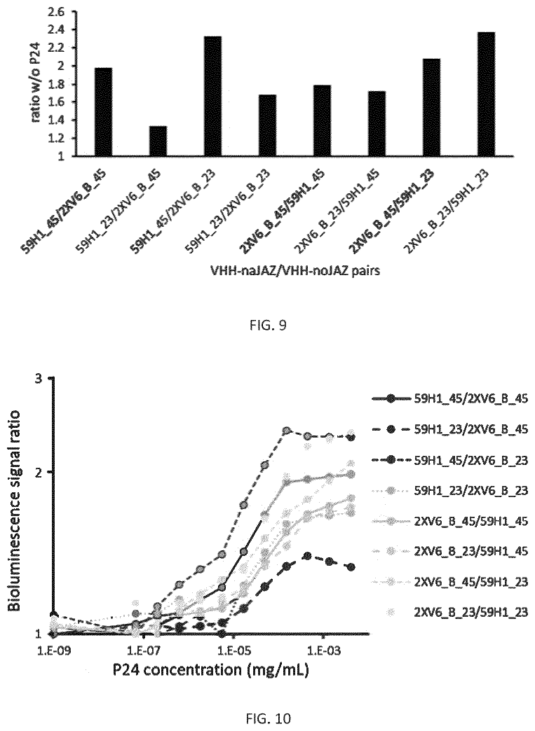

Bioluminescence (RLU/s) of the mix (VHH-linker-naJAZ 0.5 mg/mL in PBS, dilution 1/100, VHH-linker-noJAZ 0.5 mg/mL in PBS, dilution 1/700, P24 2 mg/mL, serial dilution from 1/500 then third by third, buffer PBS Tween 0.1% DTT 1 mM for a volume per well of 50 microliters) was measured in a 96-well plate. The reaction started with the substrate Hikarazine 108 5 mM in Ethanol/DMSO, dilution 1/400. It was read the relative light intensity per second along a 10 min kinetics with a luminometer Mithras-2 Berthold Results at one min after substrate addition are reported in the FIGS. 9 and 10 as well as in the Table below. The ratio of signal with and without P24 is plotted vs P24 concentration (mg/mL) in the xy-plot figure. The signal ratio value is reported in the table and the bar plot bellow for a P24 concentration of 4 mg/mL. The detection limit of P24 is 10 ng/mL in one minute.

Most of the construct pairs gives quite the same sensitivity but 59H1_45-naJAZ/2XV6_B_23-noJAZ and 2XV6_B_23-naJAZ/59H1_23-noJAZ give the best signal ratio as described in the Table below and detailed in the FIG. 9.

The first criterium for choice of pair of constructs is the highest ratio. The second criterium is the lowest ratio in the absence of target (here P24). The third criterium is the kinetic rate of signal increasing. The fourth criterium is the shortest construct. Here 59H1_45-naJAZ/2XV6_B_23-noJAZ and 2XV6_B_23-naJAZ/59H1_23-noJAZ are equivalent for the 3 first criteria, but 2XV6_B_23-naJAZ/59H1_23-noJAZ are mixing the shortest constructs. The selected pair for the LuLiFlash'P24 is 2XV6_B_23-naJAZ/59H1_23-noJAZ.

TABLE

| | | | Signal |
|---|---|---|---|
| Partner 1 | Partner 2 | Target | ratio |
| 59H1_45-naJAZ | 2XV6_B_45-noJAZ | P24 | 1.98 |
| (SEQ ID NO: 175) | (SEQ ID NO: 173) | | |
| 59H1_23-naJAZ | 2XV6_B_45-noJAZ | P24 | 1.33 |
| (SEQ ID NO: 174) | (SEQ ID NO: 173) | | |
| 59H1_45-naJAZ | 2XV6_B_23-noJAZ | P24 | 2.32 |
| (SEQ ID NO: 175) | (SEQ ID NO: 172) | | |
| 59H1_23-naJAZ | 2XV6_B-23-noJAZ | P24 | 1.68 |
| (SEQ ID NO: 174) | (SEQ ID NO: 172) | | |
| 2XV6_B_45-naJAZ | 59H1_45-noJAZ | P24 | 1.79 |
| (SEQ ID NO: 160) | (SEQ ID NO: 162) | | |
| 2XV6_B_23-naJAZ | 59H1_45-noJAZ | P24 | 1.72 |
| (SEQ ID NO: 159) | (SEQ ID NO: 162) | | |
| 2XV6_B_45-naJAZ | 59H1_23-noJAZ | P24 | 2.07 |
| (SEQ ID NO: 160) | (SEQ ID NO: 161) | | |
| 2XV6_B_23-naJAZ | 59H1_23-noJAZ | P24 | 2.37 |
| (SEQ ID NO: 159) | (SEQ ID NO: 161) | | |

1. A system for detecting an antigen comprising:
a first fusion protein comprising:
a N-terminal domain which comprises a first single domain antibody which is directed against a first epitope of said antigen
and
a C-terminal domain which comprises a first fragment of a luciferase:
wherein the first fragment has:
the amino acid sequence as set forth in SEQ ID NO: 1 or
an amino acid sequence having at least 70% amino acid sequence identity to the amino acid sequence as set forth in SEQ ID NO: 1,
wherein the first fusion protein has no luciferase activity,
and
a second fusion protein comprising:
a N-terminal domain which comprises a second single domain antibody which is directed against a second epitope of said antigen
and
a C-terminal domain which comprises a second fragment of a luciferase:
wherein the second fragment has:
the amino acid sequence as set forth in SEQ ID NO: 2 or
an amino acid sequence having at least 70% amino acid sequence identity to the amino acid sequence as set forth in SEQ ID NO: 2,
wherein the second fusion protein has no luciferase activity,
luminescence being emitted in the presence of a substrate when both the first fusion protein and the second fusion protein bind to said antigen.

2. A fusion protein comprising:
a N-terminal domain which comprises a single domain antibody which is directed against an epitope of an antigen
and
a C-terminal domain which comprises a fragment of a luciferase:
wherein the fragment has:
the amino acid sequence as set forth in SEQ ID NO: 1 or an amino acid sequence having at least 70% amino acid sequence identity to the amino acid sequence as set forth in SEQ ID NO: 1
or
the amino acid sequence as set forth in SEQ ID NO: 2 or an amino acid sequence having at least 70% amino acid sequence identity to the amino acid sequence as set forth in SEQ ID NO: 2,
wherein the fusion protein has no luciferase activity.

3. A complex comprising:
a first fusion protein comprising:
a N-terminal domain which comprises a first single domain antibody which is directed against a first epitope of an antigen
and
a C-terminal domain which comprises a first fragment of a luciferase, wherein the first fragment has:
the amino acid sequence as set forth in SEQ ID NO: 1 or
an amino acid sequence having at least 70% amino acid sequence identity to the amino acid sequence as set forth in SEQ ID NO: 1,
wherein the first fusion protein has no luciferase activity
a second fusion protein comprising:
a N-terminal domain which comprises a second single domain antibody which is directed against a second epitope of an antigen
and
a C-terminal domain which comprises a second fragment of a luciferase, wherein the second fragment has:
the amino acid sequence as set forth in SEQ ID NO: 2 or
an amino acid sequence having at least 70% amino acid sequence identity to the amino acid sequence as set forth in SEQ ID NO: 2,
wherein the second fusion protein has no luciferase activity
and
the antigen;
the first and the second fusion proteins being both bound to the antigen.

4. The system according to claim 1, the fusion protein according to claim 2 and the complex according to claim 3 wherein:
the single domain antibody and the fragment of the luciferase are linked by a linker,
the first single domain antibody and the first fragment of the luciferase are linked by a linker, called first linker,
and/or
the second single domain antibody and the second fragment of the luciferase are linked by a linker, called second linker,
and wherein the linker, the first linker and/or the second linker have the amino acid sequence selected from the group consisting of G, GS, GₙSₚ with n=1 to 5 and p=1 to 3, SGnSp with n=1 to 5 and p=0 to 3, SEQ ID NO: 102, SEQ ID NO: 103, SEQ ID NO: 105 to SEQ ID NO: 108, SEQ ID NO: 110 to SEQ ID NO: 113, SEQ ID NO: 124 and SEQ ID NO: 140 to 154, or a variant thereof.

5. The system according to claim 1 or 4, the fusion protein according to claim 2 or 4 and the complex according to claim 3 or 4 wherein the luciferase has:
an amino acid sequence selected from the group consisting of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 and SEQ ID NO: 19 or
an amino acid sequence having at least 70% amino acid sequence identity to the amino acid sequence selected from the group consisting of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 and SEQ ID NO: 19.

6. The system according to any one of claims 1 and 4-5, the fusion protein according to any one of claims 2 and 4-5 and the complex according to any one of claims 3 to 5 wherein the single domain antibody, the first single domain antibody and/or the second single domain antibody is a variable domain of a camelid heavy-chain antibody (VHH).

7. The system according to any one of claims 1 and 4-6, the fusion protein according to any one of claims 2 and 4-6 and the complex according to any one of claims 3 to 6 wherein the single domain antibody, the first single domain antibody and/or the second single domain antibody are directed against a N protein, preferably a N protein of SARS-CoV-2.

8. The system according to claim 7, the fusion protein according to claim 7 and the complex according to claim 7 wherein the single domain antibody, the first single domain antibody and/or the second single domain antibody is VHH and comprises:
an amino acid sequence selected from the group consisting of SEQ ID NO: 20 to 29 or
an amino acid sequence that is at least 80% identical to an amino acid sequence selected from the group consisting of SEQ ID NO: 20 to 29.

9. The system according to any one of claims 1 and 4-6, the fusion protein according to any one of claims 2 and 4-6 and the complex according to any one of claims 3 to 6 wherein the single domain antibody, the first single domain antibody and/or the second single domain antibody are directed against a S protein, preferably a S protein of SARS-CoV-2.

10. The system according to claim 8, the fusion protein according to claim 9 and the complex according to claim 9 wherein the single domain antibody, the first single domain antibody and/or the second single domain antibody is a VHH and comprises:
an amino acid sequence selected from the group consisting of SEQ ID NO: 78, SEQ ID NO: 79, SEQ ID NO: 80, SEQ ID NO: 126, SEQ ID NO: 127, SEQ ID NO: 128, SEQ ID NO: 129 and SEQ ID NO: 130 or
an amino acid sequence that is at least 80% identical to an amino acid sequence selected from the group consisting of SEQ ID NO: 78, SEQ ID NO: 79 SEQ ID NO: 80, SEQ ID NO: 126, SEQ ID NO: 127, SEQ ID NO: 128, SEQ ID NO: 129 and SEQ ID NO: 130.

11. The system according to any one of claims 1 and 4-6, the fusion protein according to any one of claims 2 and 4-6 and the complex according to any one of claims 3 to 6 wherein the single domain antibody, the first single domain antibody and/or the second single domain antibody are directed against P24, preferably P24 having the amino acid sequence SEQ ID NO: 155.

12. The system according to claim 11, the fusion protein according to claim 11 and the complex according to claim 11 wherein the single domain antibody, the first single domain antibody and/or the second single domain antibody is VHH and comprises:
an amino acid sequence SEQ ID NO: 156 or 157 or
an amino acid sequence that is at least 80% identical to an amino acid sequence SEQ ID NO: 156 or 157.

13. A polynucleotide encoding the fusion protein as defined in any one of claims 2 to 12, the first fusion protein as defined in any one of claims 1 and 3 to 12 and/or the second fusion protein as defined in any one of claims 1 and 3 to 12.

14. A vector comprising the polynucleotide according to claim 13.

15. A cell comprising the polynucleotide according to claim 13 or the vector according to claim 14.

16. A kit comprising:
the system according to any one of claims 1 and 4 to 12 and
a substrate for the luciferase.

17. The kit according to claim 16 wherein the substrate is 8-(2,3-difluorobenzyl)-2-((5-methylfuran-2-yl)methyl)-6-phenylimidazo[1,2-a]pyrazin-3(7H)-one.

18. The use of the system according to any one of claims 1 and 4 to 12 for detecting and/or quantifying the antigen in a sample.

19. A method for detecting and/or quantifying the presence of an antigen in a sample comprising the steps of:
(a) contacting the sample with the system according to any one of claims 1 and 4 to 12 and a substrate of the luciferase,
(b) detecting and/or quantifying the luminescence and/or the increasing rate of luminescence.

20. The use according to claim 18 or the method according to claim 19 wherein the sample is selected from the body fluid group consisting of serum, saliva, rhino-pharyngeal or nasal swab wash, urine, feces smear, cell culture supernatant, cell lysate and sewer fluid or solid.


SEQUENCE LISTING (175 sequences)

SEQ ID NO: 1 - protein, 83 aa, synthetic construct
  REGION 1..83: note = fragment of luciferase
FTLEDFVGDWRQTAGRNLDQVLEQGGVSSLFQNLGVSVTPIQRIVKSGENGLKIDIHVIIPYEGLSGDQMGQIEKIFKVVYPV

SEQ ID NO: 2 - protein, 86 aa, synthetic construct
  REGION 1..86: note = Fragment of luciferase
DDHHFKVILHYGTLVIDGVTPNMIDYFGRPFEGIAVFDGKKITVTGTLENGNKIIDERLINPDGSLLFRVTINGVTGERLSERILA

SEQ ID NO: 3 - protein, 171 aa, synthetic construct
  REGION 1..171: note = luciferase
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 4 - protein, 171 aa, synthetic construct
  REGION 1..171: note = luciferase
MVFTLEDFVGDWRQTAGRNLDQVLEQGGVSSLFQNLGVSVTPIQRIVKSGENGLKIDIHVIIPYEGLSGDQMGQIEKIFKVVYPVDDHHFKVILHYGTLVIDGVTPNMIDYFGRPFEGIAVFDGKKITVTGTLENGNKIIDERLINPDGSLLFRVTINGVTGERLSERILA

SEQ ID NO: 5 - protein, 171 aa, synthetic construct
  REGION 1..171: note = luciferase
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPFEGIAVFDGKKITVTGTLWNGNKIIDERLINPDGSLLFRVTINGVTGWRLCERILA

SEQ ID NO: 6 - protein, 171 aa, synthetic construct
  REGION 1..171: note = luciferase
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLTNGNKIIDERLINPDGSLLFRVTINGVTGWRLCERILA

SEQ ID NO: 7 - protein, 171 aa, synthetic construct
  REGION 1..171: note = luciferase
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGTRLCERILA

SEQ ID NO: 8 - protein, 171 aa, synthetic construct
  REGION 1..171: note = luciferase
MVFTLEDFVGDWRQTAGYNLDQVLEQGGVSSLFQNLGVSVTPIQRIVLSGENGLKADIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 9 - protein, 171 aa, synthetic construct
  REGION 1..171: note = luciferase
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPFEGIAVFDGKKITVTGTLTNGNKIIDERLINPDGSLLFRVTINGVTGWRLCERILA

SEQ ID NO: 10 - protein, 171 aa, synthetic construct
  REGION 1..171: note = luciferase
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPFEGIAVFDGKKITVTGTLWNGNKIIDERLINPDGSLLFRVTINGVTGWRLSERILA

SEQ ID NO: 11 - protein, 171 aa, synthetic construct
  REGION 1..171: note = luciferase
MVFTLEDFVGDWRQTAGRNLDQVLEQGGVSSLFQNLGVSVTPIQRIVKSGENGLKIDIHVIIPYEGLSGDQMGQIEKIFKVVYPVDDHHFKVILHYGTLVIDGVTPNMIDYFGRPYEGIAVFDGKKITVTGTLENGNKIIDERLINPDGSLLFRVTINGVTGERLCERILA

SEQ ID NO: 12 - protein, 171 aa, synthetic construct
  REGION 1..171: note = luciferase
MVFTLEDFVGDWRQTAGRNLDQVLEQGGVSSLFQNLGVSVTPIQRIVKSGENGLKIDIHVIIPYEGLSGDQMGQIEKIFKVVYPVDDHHFKVILHYGTLVIDGVTPNMIDYFGRPYEGIAVFDGKKITVTGTLENGNKIIDERLINPDGSLLFRVTINGVTGERLSERILA

SEQ ID NO: 13 - protein, 171 aa, synthetic construct
  REGION 1..171: note = luciferase
MVFTLEDFVGDWRQTAGRNLDQVLEQGGVSSLFQNLGVSVTPIQRIVKSGENGLKIDIHVIIPYEGLSGDQMGQIEKIFKVVYPVDDHHFKVILHYGTLVIDGVTPNMIDYFGRPFEGIAVFDGKKITVTGTLENGNKIIDERLINPDGSLLFRVTINGVTGERLCERILA

SEQ ID NO: 14 - protein, 171 aa, synthetic construct
  REGION 1..171: note = luciferase
MVFTLEDFVGDWRQTAGRNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 15 - protein, 171 aa, synthetic construct
  REGION 1..171: note = luciferase
MVFTLEDFVGDWRQTAGYNLDQVLEQGGVSSLFQNLGVSVTPIQRIVKSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 16 - protein, 171 aa, synthetic construct
  REGION 1..171: note = luciferase
MVFTLEDFVGDWRQTAGYNLDQVLEQGGVSSLFQNLGVSVTPIQRIVLSGENGLKADIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGTRLCERILA

SEQ ID NO: 17 - protein, 171 aa, synthetic construct
  REGION 1..171: note = luciferase
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLENGNKIIDERLINPDGSLLFRVTINGVTGWRLCERILA

SEQ ID NO: 18 - protein, 171 aa, synthetic construct
  REGION 1..171: note = luciferase
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGERLCERILA

SEQ ID NO: 19 - protein, 171 aa, synthetic construct
  REGION 1..171: note = luciferase
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLSERILA

SEQ ID NO: 20 - protein, 123 aa, synthetic construct
  REGION 1..123: note = VHH
EVQLVESGGGLVQPGGSLRLSCTVSEFSLRWNAIGWFRQAPGKEREGVSCISSNGAYTYIADSVKGRFAISTDSVKKMVYLQMNMLKPEDTAVYYCATGSPGCYSAVDEFPYWGRGTQVTVSS

SEQ ID NO: 21 - protein, 125 aa, synthetic construct
  REGION 1..125: note = VHH
EVQLVESGGGLVQAGGSLRLSCAASGRTFSSYAMGWFRQAPGKEREFVAAIGWMVGSIYYADSVKDRFTISRDNAKNTVYLQMNSLKPEDTAVYYCAAELGGSYLSWRDYGMDYWGKGTLVTVSS

SEQ ID NO: 22 - protein, 129 aa, synthetic construct
  REGION 1..129: note = VHH
EVQLVESGGGLVQAGDSLRLSCAASGRTFSNYAMGWFRQAPGKEEREFVAAISRDGGFKFYAESVKGRFTISRDIARDTVYLQMNSLKPEDTAVYYCAAKSNTYFSDGIITSRTQYDYWGQGTQVTVSS

SEQ ID NO: 23 - protein, 128 aa, synthetic construct
  REGION 1..128: note = VHH
EVQLVESGGGLVEPGGSLRLSCAASGFTWDYYDIGWFRQAPGKEREGVACISSSGSSTNYGDSVKGRFTISRDNAKKTVYLQMNSLKPEDTAVYYCAADIVDYGLESASCMWIDRGYWGQGTQVTVSS

SEQ ID NO: 24 - protein, 125 aa, synthetic construct
  REGION 1..125: note = VHH
EVQLVESGGGLVQPGGSLRLSCAASGFGLDYYAIGWFRQAPGKEREGVSCISNSGRSTNPADSVKGRFTISRDNAKNTVYLQMNSLKPEDTAVYYCAATAWRHACTHISNEYDYWGQGTQVTVSS

SEQ ID NO: 25 - protein, 128 aa, synthetic construct
  REGION 1..128: note = VHH
EVQLQASGGGLVQPGGSLRLSCAASGFTLGYYRIGWFRQAPGKEREGVSCLSSSGRSTNYADSVKGRFTISTDNAKNTVYLQMDSLKPEDTAVYYCAADFTPGPRLCSILSLNEYSAWGQGTQVTVSS

SEQ ID NO: 26 - protein, 127 aa, synthetic construct
  REGION 1..127: note = VHH
EVQLVESGGGLVQPGGSLRLSCAASGFTSDYYVIGWFRQAPGKEREGVSCISSGGGSTNYADSVKGRFTISRDNAKNTVYLQMNSLKPEDTAVYYCAALNRIHYYSCSVLMGDYGSWGQGTQVTVSS

SEQ ID NO: 27 - protein, 122 aa, synthetic construct
  REGION 1..122: note = VHH
EVQLVESGGGLVQPGGSLRLSCAASGFTLDYYAIYWFRQAPGKEREGVSCISSSGGSTNYADSVKGRFTISRDNAKNTVYLQMNSLKPEDTAVYYCAAGPSECGYSDYLDYWGQGTQVTVSS

SEQ ID NO: 28 - protein, 124 aa, synthetic construct
  REGION 1..124: note = VHH
EVQLQASGGGLVQAGGSLRLSCAASGRTFSSYAMGWFREAPGKEREFVAAISWSGAGTYYADSVKGRFTISRDNAKNTVYLQMNSLKPEDTAVYYCAAPSAVVAGTYVADYDYWGQGTQVTVSS

SEQ ID NO: 29 - protein, 124 aa, synthetic construct
  REGION 1..124: note = VHH
QVQLVESGGGLVQAGGSLRLSCAASGRSFSNYNTAWFRQAPGKEREFVALISWTVGNTPYADSVKGRFTISRDNAKNTVYLQMNSLNAEDTAVYYCAAGRPSIYYRTYDRYDYWGQGTQVTVSS

SEQ ID NO: 30 - protein, 11 aa, synthetic construct
  REGION 1..11: note = VHH CDR1
SEFSLRWNAIG

SEQ ID NO: 31 - protein, 18 aa, synthetic construct
  REGION 1..18: note = VHH CDR2
SCISSNGAYTYIADSVKG

SEQ ID NO: 32 - protein, 16 aa, synthetic construct
  REGION 1..16: note = VHH CDR3
ATGSPGCYSAVDEFPY

SEQ ID NO: 33 - protein, 11 aa, synthetic construct
  REGION 1..11: note = VHH CDR1
SGRTFSSYAMG

SEQ ID NO: 34 - protein, 18 aa, synthetic construct
  REGION 1..18: note = VHH CDR2
AAIGWMVGSIYYADSVKD

SEQ ID NO: 35 - protein, 18 aa, synthetic construct
  REGION 1..18: note = VHH CDR3
AAELGGSYLSWRDYGMDY

SEQ ID NO: 36 - protein, 11 aa, synthetic construct
  REGION 1..11: note = VHH CDR1
SGRTFSNYAMG

SEQ ID NO: 37 - protein, 18 aa, synthetic construct
  REGION 1..18: note = VHH CDR2
AAISRDGGFKFYAESVKG

SEQ ID NO: 38 - protein, 21 aa, synthetic construct
  REGION 1..21: note = VHH CDR3
AAKSNTYFSDGIITSRTQYDY

SEQ ID NO: 39 - protein, 11 aa, synthetic construct
  REGION 1..11: note = VHH CDR1
SGFTWDYYDIG

SEQ ID NO: 40 - protein, 18 aa, synthetic construct
  REGION 1..18: note = VHH CDR2
ACISSSGSSTNYGDSVKG

SEQ ID NO: 41 - protein, 21 aa, synthetic construct
  REGION 1..21: note = VHH CDR3
AADIVDYGLESASCMWIDRGY

SEQ ID NO: 42 - protein, 11 aa, synthetic construct
  REGION 1..11: note = VHH CDR1
SGFGLDYYAIG

SEQ ID NO: 43 - protein, 18 aa, synthetic construct
  REGION 1..18: note = VHH CDR2
SCISNSGRSTNPADSVKG

SEQ ID NO: 44 - protein, 18 aa, synthetic construct
  REGION 1..18: note = VHH CDR3
AATAWRHACTHISNEYDY

SEQ ID NO: 45 - protein, 11 aa, synthetic construct
  REGION 1..11: note = SGFTLGYYRIG
SGFTLGYYRIG

SEQ ID NO: 46 - protein, 18 aa, synthetic construct
  REGION 1..18: note = VHH CDR2
SCLSSSGRSTNYADSVKG

SEQ ID NO: 47 - protein, 21 aa, synthetic construct
  REGION 1..21: note = VHH CDR3
AADFTPGPRLCSILSLNEYSA

SEQ ID NO: 48 - protein, 11 aa, synthetic construct
  REGION 1..11: note = VHH CDR1
SGFTSDYYVIG

SEQ ID NO: 49 - protein, 18 aa, synthetic construct
  REGION 1..18: note = VHH CDR2
SCISSGGGSTNYADSVKG

SEQ ID NO: 50 - protein, 20 aa, synthetic construct
  REGION 1..20: note = VHH CDR3
AALNRIHYYSCSVLMGDYGS

SEQ ID NO: 51 - protein, 11 aa, synthetic construct
  REGION 1..11: note = VHH CDR1
SGFTLDYYAIY

SEQ ID NO: 52 - protein, 18 aa, synthetic construct
  REGION 1..18: note = VHH CDR2
SCISSSGGSTNYADSVKG

SEQ ID NO: 53 - protein, 15 aa, synthetic construct
  REGION 1..15: note = VHH CDR3
AAGPSECGYSDYLDY

SEQ ID NO: 54 - protein, 11 aa, synthetic construct
  REGION 1..11: note = VHH CDR1
SGRTFSSYAMG

SEQ ID NO: 55 - protein, 18 aa, synthetic construct
  REGION 1..18: note = VHH CDR2
AAISWSGAGTYYADSVKG

SEQ ID NO: 56 - protein, 17 aa, synthetic construct
  REGION 1..17: note = VHH CDR3
AAPSAVVAGTYVADYDY

SEQ ID NO: 57 - protein, 11 aa, synthetic construct
  REGION 1..11: note = VHH CDR1
SGRSFSNYNTA

SEQ ID NO: 58 - protein, 18 aa, synthetic construct
  REGION 1..18: note = VHH CDR2
ALISWTVGNTPYADSVKG

SEQ ID NO: 59 - protein, 17 aa, synthetic construct
  REGION 1..17: note = VHH CDR3
AAGRPSIYYRTYDRYDY

SEQ ID NO: 60 - protein, 6 aa, synthetic construct
  REGION 1..6: note = Tag
HHHHHH

SEQ ID NO: 61 - protein, 15 aa, synthetic construct
  REGION 1..15: note = Tag
GLNDIFEAQKIEWHE

SEQ ID NO: 62 - protein, 37 aa, synthetic construct
  REGION 1..37: note = Tag
DEKTTGWRGGHVVEGLAGELEQLRARLEHHPQGQREP

SEQ ID NO: 63 - protein, 28 aa, synthetic construct
  REGION 1..28: note = Tag
WSHPQFEKGGGSGGGSGGSAWSHPQFEK

SEQ ID NO: 64 - protein, 9 aa, synthetic construct
  REGION 1..9: note = Tag
YPYDVPDYA

SEQ ID NO: 65 - protein, 10 aa, synthetic construct
  REGION 1..10: note = Tag
EQKLISEEDL

SEQ ID NO: 66 - protein, 266 aa, synthetic construct
  REGION 1..266: note = Fusion protein
MAEVQLVESGGGLVEPGGSLRLSCAASGFTWDYYDIGWFRQAPGKEREGVACISSSGSSTNYGDSVKGRFTISRDNAKKTVYLQMNSLKPEDTAVYYCAADIVDYGLESASCMWIDRGYWGQGTQVTVSSAAAGEMETSQNPGEEKPQASPEGRPESETSCLVTTTDNQISTEQGFTLEDFVGDWRQTAGRNLDQVLEQGGVSSLFQNLGVSVTPIQRIVKSGENGLKIDIHVIIPYEGLSGDQMGQIEKIFKVVYPVLEHHHHHH

SEQ ID NO: 67 - protein, 269 aa, synthetic construct
  REGION 1..269: note = Fusion protein
MAEVQLQASGGGLVQPGGSLRLSCAASGFTLGYYRIGWFRQAPGKEREGVSCLSSSGRSTNYADSVKGRFTISTDNAKNTVYLQMDSLKPEDTAVYYCAADFTPGPRLCSILSLNEYSAWGQGTQVTVSSAAAGEMETSQNPGEEKPQASPEGRPESETSCLVTTTDNQISTEQGDDHHFKVILHYGTLVIDGVTPNMIDYFGRPFEGIAVFDGKKITVTGTLENGNKIIDERLINPDGSLLFRVTINGVTGERLSERILALEHHHHHH

SEQ ID NO: 68 - protein, 256 aa, synthetic construct
  REGION 1..256: note = Fusion protein
EVQLVESGGGLVEPGGSLRLSCAASGFTWDYYDIGWFRQAPGKEREGVACISSSGSSTNYGDSVKGRFTISRDNAKKTVYLQMNSLKPEDTAVYYCAADIVDYGLESASCMWIDRGYWGQGTQVTVSSAAAGEMETSQNPGEEKPQASPEGRPESETSCLVTTTDNQISTEQGFTLEDFVGDWRQTAGRNLDQVLEQGGVSSLFQNLGVSVTPIQRIVKSGENGLKIDIHVIIPYEGLSGDQMGQIEKIFKVVYPV

SEQ ID NO: 69 - protein, 259 aa, synthetic construct
  REGION 1..259: note = Fusion protein
EVQLQASGGGLVQPGGSLRLSCAASGFTLGYYRIGWFRQAPGKEREGVSCLSSSGRSTNYADSVKGRFTISTDNAKNTVYLQMDSLKPEDTAVYYCAADFTPGPRLCSILSLNEYSAWGQGTQVTVSSAAAGEMETSQNPGEEKPQASPEGRPESETSCLVTTTDNQISTEQGDDHHFKVILHYGTLVIDGVTPNMIDYFGRPFEGIAVFDGKKITVTGTLENGNKIIDERLINPDGSLLFRVTINGVTGERLSERILA

SEQ ID NO: 70 - protein, 269 aa, synthetic construct
  REGION 1..269: note = Fusion protein
MAEVQLQASGGGLVQPGGSLRLSCAASGFTLGYYRIGWFRQAPGKEREGVSCLSSSGRSTNYADSVKGRFTISTDNAKNTVYLQMDSLKPEDTAVYYCAADFTPGPRLCSILSLNEYSAWGQGTQVTVSSAAAGEMETSQNPGEEKPQASPEGRPESETSCLVTTTDNQISTEQGDDHHFKVILHYGTLVIDGVTPNMIDYFGRPYEGIAVFDGKKITVTGTLENGNKIIDERLINPDGSLLFRVTINGVTGERLSERILALEHHHHHH

SEQ ID NO: 71 - protein, 259 aa, synthetic construct
  REGION 1..259: note = Fusion protein
EVQLQASGGGLVQPGGSLRLSCAASGFTLGYYRIGWFRQAPGKEREGVSCLSSSGRSTNYADSVKGRFTISTDNAKNTVYLQMDSLKPEDTAVYYCAADFTPGPRLCSILSLNEYSAWGQGTQVTVSSAAAGEMETSQNPGEEKPQASPEGRPESETSCLVTTTDNQISTEQGDDHHFKVILHYGTLVIDGVTPNMIDYFGRPYEGIAVFDGKKITVTGTLENGNKIIDERLINPDGSLLFRVTINGVTGERLSERILA

SEQ ID NO: 72 - protein, 266 aa, synthetic construct
  REGION 1..266: note = Fusion protein
MAEVQLQASGGGLVQPGGSLRLSCAASGFTLGYYRIGWFRQAPGKEREGVSCLSSSGRSTNYADSVKGRFTISTDNAKNTVYLQMDSLKPEDTAVYYCAADFTPGPRLCSILSLNEYSAWGQGTQVTVSSAAAGEMETSQNPGEEKPQASPEGRPESETSCLVTTTDNQISTEQGFTLEDFVGDWRQTAGRNLDQVLEQGGVSSLFQNLGVSVTPIQRIVKSGENGLKIDIHVIIPYEGLSGDQMGQIEKIFKVVYPVLEHHHHHH

SEQ ID NO: 73 - protein, 269 aa, synthetic construct
  REGION 1..269: note = Fusion protein
MAEVQLVESGGGLVEPGGSLRLSCAASGFTWDYYDIGWFRQAPGKEREGVACISSSGSSTNYGDSVKGRFTISRDNAKKTVYLQMNSLKPEDTAVYYCAADIVDYGLESASCMWIDRGYWGQGTQVTVSSAAAGEMETSQNPGEEKPQASPEGRPESETSCLVTTTDNQISTEQGDDHHFKVILHYGTLVIDGVTPNMIDYFGRPFEGIAVFDGKKITVTGTLENGNKIIDERLINPDGSLLFRVTINGVTGERLSERILALEHHHHHH

SEQ ID NO: 74 - protein, 256 aa, synthetic construct
  REGION 1..256: note = Fusion protein
EVQLQASGGGLVQPGGSLRLSCAASGFTLGYYRIGWFRQAPGKEREGVSCLSSSGRSTNYADSVKGRFTISTDNAKNTVYLQMDSLKPEDTAVYYCAADFTPGPRLCSILSLNEYSAWGQGTQVTVSSAAAGEMETSQNPGEEKPQASPEGRPESETSCLVTTTDNQISTEQGFTLEDFVGDWRQTAGRNLDQVLEQGGVSSLFQNLGVSVTPIQRIVKSGENGLKIDIHVIIPYEGLSGDQMGQIEKIFKVVYPV

SEQ ID NO: 75 - protein, 259 aa, synthetic construct
  REGION 1..259: note = Fusion protein
EVQLVESGGGLVEPGGSLRLSCAASGFTWDYYDIGWFRQAPGKEREGVACISSSGSSTNYGDSVKGRFTISRDNAKKTVYLQMNSLKPEDTAVYYCAADIVDYGLESASCMWIDRGYWGQGTQVTVSSAAAGEMETSQNPGEEKPQASPEGRPESETSCLVTTTDNQISTEQGDDHHFKVILHYGTLVIDGVTPNMIDYFGRPFEGIAVFDGKKITVTGTLENGNKIIDERLINPDGSLLFRVTINGVTGERLSERILA

SEQ ID NO: 76 - protein, 269 aa, synthetic construct
  REGION 1..269: note = Fusion protein
MAEVQLVESGGGLVEPGGSLRLSCAASGFTWDYYDIGWFRQAPGKEREGVACISSSGSSTNYGDSVKGRFTISRDNAKKTVYLQMNSLKPEDTAVYYCAADIVDYGLESASCMWIDRGYWGQGTQVTVSSAAAGEMETSQNPGEEKPQASPEGRPESETSCLVTTTDNQISTEQGDDHHFKVILHYGTLVIDGVTPNMIDYFGRPYEGIAVFDGKKITVTGTLENGNKIIDERLINPDGSLLFRVTINGVTGERLSERILALEHHHHHH

SEQ ID NO: 77 - protein, 259 aa, synthetic construct
  REGION 1..259: note = Fusion protein
EVQLVESGGGLVEPGGSLRLSCAASGFTWDYYDIGWFRQAPGKEREGVACISSSGSSTNYGDSVKGRFTISRDNAKKTVYLQMNSLKPEDTAVYYCAADIVDYGLESASCMWIDRGYWGQGTQVTVSSAAAGEMETSQNPGEEKPQASPEGRPESETSCLVTTTDNQISTEQGDDHHFKVILHYGTLVIDGVTPNMIDYFGRPYEGIAVFDGKKITVTGTLENGNKIIDERLINPDGSLLFRVTINGVTGERLSERILA

SEQ ID NO: 78 - protein, 129 aa, synthetic construct
  REGION 1..129: note = VHH
MAEVQLQASGGGLVEAGGSLRLSCTTSGLTFSSVTMGWFRQAPGKEREFVAAIRWKFGNLGYADSVKGRFTVSRDNAKNTVYLQMNSLKPEDTAVYYCAAARVGEIIAVLISPSNYAYWGQGTQVTVSS

SEQ ID NO: 79 - protein, 120 aa, synthetic construct
  REGION 1..120: note = VHH
MAEVQLQASGGGLVQPGGSLRLSCAASGSFFSISAMGWYRQAPGKQRELVADITSGGSTNYADSVKGRFTISRDNAKNTVYLQMNSLKPEDTAVYYCHVQVGVHPIGYDVWGQGTQVTVS

SEQ ID NO: 80 - protein, 135 aa, synthetic construct
  REGION 1..135: note = VHH
MAQVQLVESGGGLVQAGDSLRLSCAVSGRTFSSLIMGWFRQAPGKEREFVARITYSGGSTHYADSVKGRFTISRDNAKNTVYLQMNSLKPEDTAVYYCAADTRGFSWSSSGGYDYWGQGTQVTVASEPKTPKPQP

SEQ ID NO: 81 - protein, 8 aa, synthetic construct
  REGION 1..8: note = VHH CDR1
GLTFSSVT

SEQ ID NO: 82 - protein, 10 aa, synthetic construct
  REGION 1..10: note = VHH CDR2
IRWKFGNLGY

SEQ ID NO: 83 - protein, 20 aa, synthetic construct
  REGION 1..20: note = VHH CDR3
AAARVGEIIAVLISPSNYAY

SEQ ID NO: 84 - protein, 8 aa, synthetic construct
  REGION 1..8: note = VHH CDR1
GSFFSISA

SEQ ID NO: 85 - protein, 10 aa, synthetic construct
  REGION 1..10: note = VHH CDR2
ITSGGSTNYA

SEQ ID NO: 86 - protein, 13 aa, synthetic construct
  REGION 1..13: note = VHH CDR3
HVQVGVHPIGYDV

SEQ ID NO: 87 - protein, 8 aa, synthetic construct
  REGION 1..8: note = VHH CDR1
GRTFSSLI

SEQ ID NO: 88 - protein, 10 aa, synthetic construct
  REGION 1..10: note = VHH CDR2
ITYSGGSTHY

SEQ ID NO: 89 - protein, 17 aa, synthetic construct
  REGION 1..17: note = VHH CDR3
AADTRGFSWSSSGGYDY

SEQ ID NO: 90 - protein, 265 aa, synthetic construct
  REGION 1..265: note = Fusion protein
MAEVQLQASGGGLVEAGGSLRLSCTTSGLTFSSVTMGWFRQAPGKEREFVAAIRWKFGNLGYADSVKGRFTVSRDNAKNTVYLQMNSLKPEDTAVYYCAAARVGEIIAVLISPSNYAYWGQGTQVTVSSAAAGEMETSQNPGEEKPQASPEGRPESETSCLVTTTDNQISTEQGFTLEDFVGDWRQTAGRNLDQVLEQGGVSSLFQNLGVSVTPIQRIVKSGENGLKIDIHVIIPYEGLSGDQMGQIEKIFKVVYPVLEHHHHHH

SEQ ID NO: 91 - protein, 260 aa, synthetic construct
  REGION 1..260: note = Fusion protein
MAEVQLQASGGGLVQPGGSLRLSCAASGSFFSISAMGWYRQAPGKQRELVADITSGGSTNYADSVKGRFTISRDNAKNTVYLQMNSLKPEDTAVYYCHVQVGVHPIGYDVWGQGTQVTVSSAAAGEMETSQNPGEEKPQASPEGRPESETSCLVTTTDNQISTEQGDDHHFKVILHYGTLVIDGVTPNMIDYFGRPYEGIAVFDGKKITVTGTLENGNKIIDERLINPDGSLLFRVTINGVTGERLSERILALEHHHHHH

SEQ ID NO: 92 - protein, 256 aa, synthetic construct
  REGION 1..256: note = Fusion protein
MAEVQLQASGGGLVQPGGSLRLSCAASGSFFSISAMGWYRQAPGKQRELVADITSGGSTNYADSVKGRFTISRDNAKNTVYLQMNSLKPEDTAVYYCHVQVGVHPIGYDVWGQGTQVTVSAAAGEMETSQNPGEEKPQASPEGRPESETSCLVTTTDNQISTEQGFTLEDFVGDWRQTAGRNLDQVLEQGGVSSLFQNLGVSVTPIQRIVKSGENGLKIDIHVIIPYEGLSGDQMGQIEKIFKVVYPVLEHHHHHH

SEQ ID NO: 93 - protein, 259 aa, synthetic construct
  REGION 1..259: note = Fusion protein
MAEVQLQASGGGLVQPGGSLRLSCAASGSFFSISAMGWYRQAPGKQRELVADITSGGSTNYADSVKGRFTISRDNAKNTVYLQMNSLKPEDTAVYYCHVQVGVHPIGYDVWGQGTQVTVSAAAGEMETSQNPGEEKPQASPEGRPESETSCLVTTTDNQISTEQGDDHHFKVILHYGTLVIDGVTPNMIDYFGRPFEGIAVFDGKKITVTGTLENGNKIIDERLINPDGSLLFRVTINGVTGERLSERILALEHHHHHH

SEQ ID NO: 94 - protein, 271 aa, synthetic construct
  REGION 1..271: note = Fusion protein
MAQVQLVESGGGLVQAGDSLRLSCAVSGRTFSSLIMGWFRQAPGKEREFVARITYSGGSTHYADSVKGRFTISRDNAKNTVYLQMNSLKPEDTAVYYCAADTRGFSWSSSGGYDYWGQGTQVTVASEPKTPKPQPAAAGEMETSQNPGEEKPQASPEGRPESETSCLVTTTDNQISTEQGFTLEDFVGDWRQTAGRNLDQVLEQGGVSSLFQNLGVSVTPIQRIVKSGENGLKIDIHVIIPYEGLSGDQMGQIEKIFKVVYPVLEHHHHHH

SEQ ID NO: 95 - protein, 268 aa, synthetic construct
  REGION 1..268: note = Fusion protein
MAEVQLQASGGGLVEAGGSLRLSCTTSGLTFSSVTMGWFRQAPGKEREFVAAIRWKFGNLGYADSVKGRFTVSRDNAKNTVYLQMNSLKPEDTAVYYCAAARVGEIIAVLISPSNYAYWGQGTQVTVSSAAAGEMETSQNPGEEKPQASPEGRPESETSCLVTTTDNQISTEQGDDHHFKVILHYGTLVIDGVTPNMIDYFGRPYEGIAVFDGKKITVTGTLENGNKIIDERLINPDGSLLFRVTINGVTGERLSERILALEHHHHHH

SEQ ID NO: 96 - protein, 268 aa, synthetic construct
  REGION 1..268: note = Fusion protein
MAEVQLQASGGGLVEAGGSLRLSCTTSGLTFSSVTMGWFRQAPGKEREFVAAIRWKFGNLGYADSVKGRFTVSRDNAKNTVYLQMNSLKPEDTAVYYCAAARVGEIIAVLISPSNYAYWGQGTQVTVSSAAAGEMETSQNPGEEKPQASPEGRPESETSCLVTTTDNQISTEQGDDHHFKVILHYGTLVIDGVTPNMIDYFGRPFEGIAVFDGKKITVTGTLENGNKIIDERLINPDGSLLFRVTINGVTGERLSERILALEHHHHHH

SEQ ID NO: 97 - protein, 255 aa, synthetic construct
  REGION 1..255: note = Fusion protein
EVQLQASGGGLVEAGGSLRLSCTTSGLTFSSVTMGWFRQAPGKEREFVAAIRWKFGNLGYADSVKGRFTVSRDNAKNTVYLQMNSLKPEDTAVYYCAAARVGEIIAVLISPSNYAYWGQGTQVTVSSAAAGEMETSQNPGEEKPQASPEGRPESETSCLVTTTDNQISTEQGFTLEDFVGDWRQTAGRNLDQVLEQGGVSSLFQNLGVSVTPIQRIVKSGENGLKIDIHVIIPYEGLSGDQMGQIEKIFKVVYPV

SEQ ID NO: 98 - protein, 250 aa, synthetic construct
  REGION 1..250: note = Fusion protein
EVQLQASGGGLVQPGGSLRLSCAASGSFFSISAMGWYRQAPGKQRELVADITSGGSTNYADSVKGRFTISRDNAKNTVYLQMNSLKPEDTAVYYCHVQVGVHPIGYDVWGQGTQVTVSSAAAGEMETSQNPGEEKPQASPEGRPESETSCLVTTTDNQISTEQGDDHHFKVILHYGTLVIDGVTPNMIDYFGRPYEGIAVFDGKKITVTGTLENGNKIIDERLINPDGSLLFRVTINGVTGERLSERILA

SEQ ID NO: 99 - protein, 246 aa, synthetic construct
  REGION 1..246: note = Fusion protein
EVQLQASGGGLVQPGGSLRLSCAASGSFFSISAMGWYRQAPGKQRELVADITSGGSTNYADSVKGRFTISRDNAKNTVYLQMNSLKPEDTAVYYCHVQVGVHPIGYDVWGQGTQVTVSAAAGEMETSQNPGEEKPQASPEGRPESETSCLVTTTDNQISTEQGFTLEDFVGDWRQTAGRNLDQVLEQGGVSSLFQNLGVSVTPIQRIVKSGENGLKIDIHVIIPYEGLSGDQMGQIEKIFKVVYPV

SEQ ID NO: 100 - protein, 249 aa, synthetic construct
  REGION 1..249: note = Fusion protein
EVQLQASGGGLVQPGGSLRLSCAASGSFFSISAMGWYRQAPGKQRELVADITSGGSTNYADSVKGRFTISRDNAKNTVYLQMNSLKPEDTAVYYCHVQVGVHPIGYDVWGQGTQVTVSAAAGEMETSQNPGEEKPQASPEGRPESETSCLVTTTDNQISTEQGDDHHFKVILHYGTLVIDGVTPNMIDYFGRPFEGIAVFDGKKITVTGTLENGNKIIDERLINPDGSLLFRVTINGVTGERLSERILA

SEQ ID NO: 101 - protein, 261 aa, synthetic construct
  REGION 1..261: note = Fusion protein
QVQLVESGGGLVQAGDSLRLSCAVSGRTFSSLIMGWFRQAPGKEREFVARITYSGGSTHYADSVKGRFTISRDNAKNTVYLQMNSLKPEDTAVYYCAADTRGFSWSSSGGYDYWGQGTQVTVASEPKTPKPQPAAAGEMETSQNPGEEKPQASPEGRPESETSCLVTTTDNQISTEQGFTLEDFVGDWRQTAGRNLDQVLEQGGVSSLFQNLGVSVTPIQRIVKSGENGLKIDIHVIIPYEGLSGDQMGQIEKIFKVVYPV

SEQ ID NO: 102 - protein, 45 aa, synthetic construct
  REGION 1..45: note = Linker
AAAGEMETSQNPGEEKPQASPEGRPESETSCLVTTTDNQISTEQG

SEQ ID NO: 103 - protein, 90 aa, synthetic construct
  REGION 1..90: note = Linker
AAAGEMETSQNPGEEKPQASPEGRPESETSCLVTTTDNQISTEQGAAAGEMETSQNPGEEKPQASPEGRPESETSCLVTTTDNQISTEQG

SEQ ID NO: 104 - skipped

SEQ ID NO: 105 - protein, 16 aa, synthetic construct
  REGION 1..16: note = Linker
  REGION 2..5: note = MISC_FEATURE - X is G or nothing
  REGION 7..8: note = MISC_FEATURE - X is S or nothing
  REGION 10..13: note = MISC_FEATURE - X is G or nothing
  REGION 15..16: note = MISC_FEATURE - X is S or nothing
GXXXXSXXGXXXXSXX

SEQ ID NO: 106 - protein, 24 aa, synthetic construct
  REGION 1..24: note = Linker
  REGION 2..5: note = MISC_FEATURE - X is G or nothing
  REGION 7..8: note = MISC_FEATURE - X is S or nothing
  REGION 10..13: note = MISC_FEATURE - X is G or nothing
  REGION 15..16: note = MISC_FEATURE - X is S or nothing
  REGION 18..21: note = MISC_FEATURE - X is G or nothing
  REGION 23..24: note = MISC_FEATURE - X is S or nothing
GXXXXSXXGXXXXSXXGXXXXSXX

SEQ ID NO: 107 - protein, 32 aa, synthetic construct
  REGION 1..32: note = Linker
  REGION 2..5: note = MISC_FEATURE - X is G or nothing
  REGION 7..8: note = MISC_FEATURE - X is S or nothing
  REGION 10..13: note = MISC_FEATURE - X is G or nothing
  REGION 15..16: note = MISC_FEATURE - X is S or nothing
  REGION 18..21: note = MISC_FEATURE - X is G or nothing
  REGION 23..24: note = MISC_FEATURE - X is S or nothing
  REGION 26..29: note = MISC_FEATURE - X is G or nothing
  REGION 31..32: note = MISC_FEATURE - X is S or nothing
GXXXXSXXGXXXXSXXGXXXXSXXGXXXXSXX

SEQ ID NO: 108 - protein, 40 aa, synthetic construct
  REGION 1..40: note = Linker
  REGION 2..5: note = MISC_FEATURE - X is G or nothing
  REGION 7..8: note = MISC_FEATURE - X is S or nothing
  REGION 10..13: note = MISC_FEATURE - X is G or nothing
  REGION 15..16: note = MISC_FEATURE - X is S or nothing
  REGION 18..21: note = MISC_FEATURE - X is G or nothing
  REGION 23..24: note = MISC_FEATURE - X is S or nothing
  REGION 26..29: note = MISC_FEATURE - X is G or nothing
  REGION 31..32: note = MISC_FEATURE - X is S or nothing
  REGION 34..37: note = MISC_FEATURE - X is G or nothing
  REGION 39..40: note = MISC_FEATURE - X is s or nothing
GXXXXSXXGXXXXSXXGXXXXSXXGXXXXSXXGXXXXSXX

SEQ ID NO: 109 - skipped

SEQ ID NO: 110 - protein, 17 aa, synthetic construct
  REGION 1..17: note = Linker
  REGION 3..6: note = MISC_FEATURE - X is G or nothing
  REGION 8..9: note = MISC_FEATURE - X is S or nothing
  REGION 11..14: note = MISC_FEATURE - X is G or nothing
  REGION 16..17: note = MISC_FEATURE - X is S or nothing
SGXXXXSXXGXXXXSXX

SEQ ID NO: 111 - protein, 25 aa, synthetic construct
  REGION 1..25: note = Linker
  REGION 3..6: note = MISC_FEATURE - X is G or nothing
  REGION 8..9: note = MISC_FEATURE - X is S or nothing
  REGION 11..14: note = MISC_FEATURE - X is G or nothing
  REGION 16..17: note = MISC_FEATURE - X is S or nothing
  REGION 19..22: note = MISC_FEATURE - X is G or nothing
  REGION 24..25: note = MISC_FEATURE - X is S or nothing
SGXXXXSXXGXXXXSXXGXXXXSXX

SEQ ID NO: 112 - protein, 33 aa, synthetic construct
  REGION 1..33: note = Linker
  REGION 3..6: note = MISC_FEATURE - X is G or nothing
  REGION 8..9: note = MISC_FEATURE - X is S or nothing
  REGION 11..14: note = MISC_FEATURE - X is G or nothing
  REGION 16..17: note = MISC_FEATURE - X is S or nothing
  REGION 19..22: note = MISC_FEATURE - X is G or nothing
  REGION 24..25: note = MISC_FEATURE - X is S or nothing
  REGION 27..30: note = MISC_FEATURE - X is G or nothing
  REGION 32..33: note = MISC_FEATURE - X is S or nothing
SGXXXXSXXGXXXXSXXGXXXXSXXGXXXXSXX

SEQ ID NO: 113 - protein, 41 aa, synthetic construct
  REGION 1..41: note = Linker
  REGION 3..6: note = MISC_FEATURE - X is G or nothing
  REGION 8..9: note = MISC_FEATURE - X is S or nothing
  REGION 11..14: note = MISC_FEATURE - X is G or nothing
  REGION 16..17: note = MISC_FEATURE - X is S or nothing
  REGION 19..22: note = MISC_FEATURE - X is G or nothing
  REGION 24..25: note = MISC_FEATURE - X is S or nothing
  REGION 27..30: note = MISC_FEATURE - X is G or nothing
  REGION 32..33: note = MISC_FEATURE - X is S or nothing
  REGION 35..38: note = MISC_FEATURE - X is G or nothing
  REGION 40..41: note = MISC_FEATURE - X is S or nothing
SGXXXXSXXGXXXXSXXGXXXXSXXGXXXXSXXGXXXXSXX

SEQ ID NO: 114 - protein, 86 aa, synthetic construct
  REGION 1..86: note = Fragment of luciferase
DDHHFKVILHYGTLVIDGVTPNMIDYFGRPYEGIAVFDGKKITVTGTLENGNKIIDERLINPDGSLLFRVTINGVTGERLSERILA

SEQ ID NO: 115 - protein, 258 aa, synthetic construct
  REGION 1..258: note = Fusion protein
EVQLQASGGGLVEAGGSLRLSCTTSGLTFSSVTMGWFRQAPGKEREFVAAIRWKFGNLGYADSVKGRFTVSRDNAKNTVYLQMNSLKPEDTAVYYCAAARVGEIIAVLISPSNYAYWGQGTQVTVSSAAAGEMETSQNPGEEKPQASPEGRPESETSCLVTTTDNQISTEQGDDHHFKVILHYGTLVIDGVTPNMIDYFGRPFEGIAVFDGKKITVTGTLENGNKIIDERLINPDGSLLFRVTINGVTGERLSERILA

SEQ ID NO: 116 - protein, 258 aa, synthetic construct
  REGION 1..258: note = Fusion protein
EVQLQASGGGLVEAGGSLRLSCTTSGLTFSSVTMGWFRQAPGKEREFVAAIRWKFGNLGYADSVKGRFTVSRDNAKNTVYLQMNSLKPEDTAVYYCAAARVGEIIAVLISPSNYAYWGQGTQVTVSSAAAGEMETSQNPGEEKPQASPEGRPESETSCLVTTTDNQISTEQGDDHHFKVILHYGTLVIDGVTPNMIDYFGRPYEGIAVFDGKKITVTGTLENGNKIIDERLINPDGSLLFRVTINGVTGERLSERILA

SEQ ID NO: 117 - protein, 310 aa, synthetic construct
  REGION 1..310: note = Fusion protein
MAEVQLQASGGGLVEAGGSLRLSCTTSGLTFSSVTMGWFRQAPGKEREFVAAIRWKFGNLGYADSVKGRFTVSRDNAKNTVYLQMNSLKPEDTAVYYCAAARVGEIIAVLISPSNYAYWGQGTQVTVSSAAAVFTLEDFVGDWRQTAGRNLDQVLEQGGVSSLFQNLGVSVTPIQRIVKSGENGLKIDIHVIIPYEGLSGDQMGQIEKIFKVVYPVDDHHFKVILHYGTLVIDGVTPNMIDYFGRPYEGIAVFDGKKITVTGTLENGNKIIDERLINPDGSLLFRVTINGVTGERLCERILALEHHHHHH

SEQ ID NO: 118 - protein, 311 aa, synthetic construct
  REGION 1..311: note = Fusion protein
MAEVQLVESGGGLVEPGGSLRLSCAASGFTWDYYDIGWFRQAPGKEREGVACISSSGSSTNYGDSVKGRFTISRDNAKKTVYLQMNSLKPEDTAVYYCAADIVDYGLESASCMWIDRGYWGQGTQVTVSSAAAVFTLEDFVGDWRQTAGRNLDQVLEQGGVSSLFQNLGVSVTPIQRIVKSGENGLKIDIHVIIPYEGLSGDQMGQIEKIFKVVYPVDDHHFKVILHYGTLVIDGVTPNMIDYFGRPYEGIAVFDGKKITVTGTLENGNKIIDERLINPDGSLLFRVTINGVTGERLCERILALEHHHHHH

SEQ ID NO: 119 - protein, 311 aa, synthetic construct; identical to SEQ ID NO: 118
  REGION 1..311: note = Fusion protein

SEQ ID NO: 120 - protein, 178 aa, synthetic construct
  REGION 1..178: note = Fusion protein
MAEVQLVESGGGLVEPGGSLRLSCAASGFTWDYYDIGWFRQAPGKEREGVACISSSGSSTNYGDSVKGRFTISRDNAKKTVYLQMNSLKPEDTAVYYCAADIVDYGLESASCMWIDRGYWGQGTQVTVSSAAADEKTTGWRGGHVVEGLAGELEQLRARLEHHPQGQREPLEHHHHHH

SEQ ID NO: 121 - protein, 391 aa, synthetic construct
  REGION 1..391: note = Fusion protein
MYRMQLLSCIALSLALVTNSASMAEVQLVESGGGLVEPGGSLRLSCAASGFTWDYYDIGWFRQAPGKEREGVACISSSGSSTNYGDSVKGRFTISRDNAKKTVYLQMNSLKPEDTAVYYCAADIVDYGLESASCMWIDRGYWGQGTQVTVSSLEVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHHV

SEQ ID NO: 122 - protein, 390 aa, synthetic construct
  REGION 1..390: note = Fusion protein
MYRMQLLSCIALSLALVTNSASMAEVQLQASGGGLVEAGGSLRLSCTTSGLTFSSVTMGWFRQAPGKEREFVAAIRWKFGNLGYADSVKGRFTVSRDNAKNTVYLQMNSLKPEDTAVYYCAAARVGEIIAVLISPSNYAYWGQGTQVTVSSLEVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHHV

SEQ ID NO: 123 - protein, 22 aa, synthetic construct
  REGION 1..22: note = Signal peptide
MYRMQLLSCIALSLALVTNSAS

SEQ ID NO: 124 - protein, 15 aa, synthetic construct
  REGION 1..15: note = Linker
LEVRSDKTHTCPPCP

SEQ ID NO: 125 - protein, 217 aa, synthetic construct
  REGION 1..217: note = Fc-IgG
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 130 aa, synthetic construct
  REGION 1..130: note = VHH
MAEVQLVESGGGLVQAGGSLRLSCAASGRAFSRYFMGWFRQAPGKEREFVAGISRSGGSTDYANFVKGRFTISRDNAKNTVYLQMNSLKPEDTAVYYCAATVDYSGTLTAARGREDYDDWGQGIQVTVSS

SEQ ID NO: 127 - protein, 123 aa, synthetic construct
  REGION 1..123: note = VHH
MAEVQLVESGGGTVQPGGSLRLSCEVSGTGFTINAMGWDRQAPGKQRELVATITRGDRIHYADSVKGRFAISRDKDKNTVYLEMNNLKPEDTAVYYCDVAAFDSSDYEVLDSWGQGTQVTVSS

SEQ ID NO: 128 - protein, 130 aa, synthetic construct
  REGION 1..130: note = VHH
MAEVQLQASGGGLVQAGGSLRLSCAAYGGTFNRYSMGWFRQAPGKEREFVARISWSVGSTKTYSDSVKGRFTISRDNAKNTVYLQMNSLKPEDTAVYYCAAARVGEIIAVLISPSNYAYWGQGTQVTVSS

SEQ ID NO: 129 - protein, 129 aa, synthetic construct
  REGION 1..129: note = VHH
MAEVQLVESGGGLVEAGGSLRLSCTTSGLTFSSVTMGWFRQAPGKEREFVAAIRWKFGNLGYADSVKGRFTVSRDNAKNTVYLQMNSLKPEDTAVYYCAAARVGEIIAVLISPSNYAYWGQGTQVTVSS

SEQ ID NO: 130 - protein, 130 aa, synthetic construct
  REGION 1..130: note = VHH
MAEVQLVESGGGLVQAGGSLRLSCAASGRAFSRYFMGWFRQAPGKEREFVAGISRSGGSTDYANFVKGRFTISRDNAKNTVELQMNSLKPEDTAVYYCAATVDYSGTLTAARGREDYDDWGQGIQVTVSS

SEQ ID NO: 131 - protein, 8 aa, synthetic construct
  REGION 1..8: note = VHH CDR1
GRAFSRYF

SEQ ID NO: 132 - protein, 10 aa, synthetic construct
  REGION 1..10: note = VHH CDR2
ISRSGGSTDY

SEQ ID NO: 133 - protein, 21 aa, synthetic construct
  REGION 1..21: note = VHH CDR3
AATVDYSGTLTAARGREDYDD

SEQ ID NO: 134 - protein, 8 aa, synthetic construct
  REGION 1..8: note = VHH CDR1
GTGFTINA

SEQ ID NO: 135 - protein, 10 aa, synthetic construct
  REGION 1..10: note = VHH CDR2
ITRGDRIHYA

SEQ ID NO: 136 - protein, 15 aa, synthetic construct
  REGION 1..15: note = VHH CDR3
DVAAFDSSDYEVLDS

SEQ ID NO: 137 - protein, 8 aa, synthetic construct
  REGION 1..8: note = VHH CDR1
GGTFNRYS

SEQ ID NO: 138 - protein, 10 aa, synthetic construct
  REGION 1..10: note = VHH CDR2
ISWSVGSTKT

SEQ ID NO: 139 - protein, 20 aa, synthetic construct
  REGION 1..20: note = VHH CDR3
AAARVGEIIAVLISPSNYAY

SEQ ID NO: 140 - protein, 59 aa, synthetic construct
AAAGEMETSQNPGEEKPQASPEGRPESETSTLVTTTDNQISTEQPGEEKPQASPEGRPG

SEQ ID NO: 141 - protein, 45 aa, synthetic construct
AAAGEMETSQNPGEEKPQASPEGRPESETSTLVTTTDNQISTEQG

SEQ ID NO: 142 - protein, 43 aa, synthetic construct
AAAGEMETSQNPGEEKPQASPEGRPESETSTLVTTTDNQISTE

SEQ ID NO: 143 - protein, 41 aa, synthetic construct
AAAGEMETSQNPGEEKPQASPEGRPESETSTLVTTTDNQIS

SEQ ID NO: 144 - protein, 39 aa, synthetic construct
AAAGEMETSQNPGEEKPQASPEGRPESETSTLVTTTDNQ

SEQ ID NO: 145 - protein, 37 aa, synthetic construct
AAAGEMETSQNPGEEKPQASPEGRPESETSTLVTTTD

SEQ ID NO: 146 - protein, 35 aa, synthetic construct
AAAGEMETSQNPGEEKPQASPEGRPESETSTLVTT

SEQ ID NO: 147 - protein, 33 aa, synthetic construct
AAAGEMETSQNPGEEKPQASPEGRPESETSTLV

SEQ ID NO: 148 - protein, 31 aa, synthetic construct
AAAGEMETSQNPGEEKPQASPEGRPESETST

SEQ ID NO: 149 - protein, 29 aa, synthetic construct
AAAGEMETSQNPGEEKPQASPEGRPESET

SEQ ID NO: 150 - protein, 27 aa, synthetic construct
AAAGEMETSQNPGEEKPQASPEGRPES

SEQ ID NO: 151 - protein, 25 aa, synthetic construct
AAAGEMETSQNPGEEKPQASPEGRP

SEQ ID NO: 152 - protein, 23 aa, synthetic construct
AAAGEMETSQNPGEEKPQASPEG

SEQ ID NO: 153 - protein, 21 aa, synthetic construct
AAAGEMETSQNPGEEKPQASP

SEQ ID NO: 154 - protein, 20 aa, synthetic construct
AAAGEMETSQNPGEEKPQAS

SEQ ID NO: 155 - protein, 70 aa, Human immunodeficiency virus 1
MADIRQGPKEPFRDYVDRFYKTLRAEQASQDVKNWMTETLLVQNANPDCKTILKALGPQATLEEMMTACQ

SEQ ID NO: 156 - protein, 115 aa, synthetic construct
MAQVQLVESGGGLVQAGGSLRLSCAASGSFFMSNVMAWYRQAPGKARELIAAIRGGDMSTVYDDSVKGRFTITRDDDKNILYLQMNDLKPEDTAMYYCKASGSSWGQGTQVTVSS

SEQ ID NO: 157 - protein, 116 aa, synthetic construct
MADVQLKESGGGLVQAGGSLRLSCAASGSISRFNAMGWWRQAPGKEREFVARIVKGFDPVLADSVKGRFTISIDSAENTLALQMNRLKPEDTAVYYCFAALDTAYWGQGTQVTVSS

SEQ ID NO: 158 - protein, 83 aa, synthetic construct
FTLEDFVGDWRQTAGRNLDQVLEQGGVSSLFQNLGVSVTPIQRIVKSGENGLKIDIHVIIPYEGLLGDQMGQIEKIFKVVYPV

SEQ ID NO: 159 - protein, 230 aa, synthetic construct
MADVQLKESGGGLVQAGGSLRLSCAASGSISRFNAMGWWRQAPGKEREFVARIVKGFDPVLADSVKGRFTISIDSAENTLALQMNRLKPEDTAVYYCFAALDTAYWGQGTQVTVSSAAAGEMETSQNPGEEKPQASPEGFTLEDFVGDWRQTAGRNLDQVLEQGGVSSLFQNLGVSVTPIQRIVKSGENGLKIDIHVIIPYEGLLGDQMGQIEKIFKVVYPVLEHHHHHH

SEQ ID NO: 160 - protein, 252 aa, synthetic construct
MADVQLKESGGGLVQAGGSLRLSCAASGSISRFNAMGWWRQAPGKEREFVARIVKGFDPVLADSVKGRFTISIDSAENTLALQMNRLKPEDTAVYYCFAALDTAYWGQGTQVTVSSAAAGEMETSQNPGEEKPQASPEGRPESETSCLVTTTDNQISTEQGFTLEDFVGDWRQTAGRNLDQVLEQGGVSSLFQNLGVSVTPIQRIVKSGENGLKIDIHVIIPYEGLLGDQMGQIEKIFKVVYPVLEHHHHHH

SEQ ID NO: 161 - protein, 232 aa, synthetic construct
MAQVQLVESGGGLVQAGGSLRLSCAASGSFFMSNVMAWYRQAPGKARELIAAIRGGDMSTVYDDSVKGRFTITRDDDKNILYLQMNDLKPEDTAMYYCKASGSSWGQGTQVTVSSAAAGEMETSQNPGEEKPQASPEGDDHHFKVILHYGTLVIDGVTPNMIDYFGRPYEGIAVFDGKKITVTGTLENGNKIIDERLINPDGSLLFRVTINGVTGERLSERILALEHHHHHH

SEQ ID NO: 162 - protein, 254 aa, synthetic construct
MAQVQLVESGGGLVQAGGSLRLSCAASGSFFMSNVMAWYRQAPGKARELIAAIRGGDMSTVYDDSVKGRFTITRDDDKNILYLQMNDLKPEDTAMYYCKASGSSWGQGTQVTVSSAAAGEMETSQNPGEEKPQASPEGRPESETSCLVTTTDNQISTEQGDDHHFKVILHYGTLVIDGVTPNMIDYFGRPYEGIAVFDGKKITVTGTLENGNKIIDERLINPDGSLLFRVTINGVTGERLSERILALEHHHHHH

SEQ ID NO: 163 - protein, 198 aa, SARS-CoV-2
NITNLCPFGEVFNATRFASVYAWNRKRISNCVADYSFLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPK

SEQ ID NO: 164 - protein, 8 aa, SARS-CoV2
YNYLYRLF

SEQ ID NO: 165 - protein, 12 aa, SARS-CoV2
VEGFNCYFPLQS

SEQ ID NO: 166 - protein, 17 aa, SARS-CoV-2
YNSASFSTFKCYGVSPT

SEQ ID NO: 167 - protein, 10 aa, SARS-CoV-2
RFASVYAWNR

SEQ ID NO: 168 - skipped

SEQ ID NO: 169 - protein, 9 aa, SARS-CoV-2
KVGGNYNYL

SEQ ID NO: 170 - protein, 5 aa, SARS-CoV-2
RDIST

SEQ ID NO: 171 - protein, 10 aa, SARS-CoV-2
FPLQSYGFQP

SEQ ID NO: 172 - protein, 233 aa, synthetic construct
MADVQLKESGGGLVQAGGSLRLSCAASGSISRFNAMGWWRQAPGKEREFVARIVKGFDPVLADSVKGRFTISIDSAENTLALQMNRLKPEDTAVYYCFAALDTAYWGQGTQVTVSSAAAGEMETSQNPGEEKPQASPEGDDHHFKVILHYGTLVIDGVTPNMIDYFGRPYEGIAVFDGKKITVTGTLENGNKIIDERLINPDGSLLFRVTINGVTGERLSERILALEHHHHHH

SEQ ID NO: 173 - protein, 255 aa, synthetic construct
MADVQLKESGGGLVQAGGSLRLSCAASGSISRFNAMGWWRQAPGKEREFVARIVKGFDPVLADSVKGRFTISIDSAENTLALQMNRLKPEDTAVYYCFAALDTAYWGQGTQVTVSSAAAGEMETSQNPGEEKPQASPEGRPESETSCLVTTTDNQISTEQGDDHHFKVILHYGTLVIDGVTPNMIDYFGRPYEGIAVFDGKKITVTGTLENGNKIIDERLINPDGSLLFRVTINGVTGERLSERILALEHHHHHH

SEQ ID NO: 174 - protein, 229 aa, synthetic construct
MAQVQLVESGGGLVQAGGSLRLSCAASGSFFMSNVMAWYRQAPGKARELIAAIRGGDMSTVYDDSVKGRFTITRDDDKNILYLQMNDLKPEDTAMYYCKASGSSWGQGTQVTVSSAAAGEMETSQNPGEEKPQASPEGFTLEDFVGDWRQTAGRNLDQVLEQGGVSSLFQNLGVSVTPIQRIVKSGENGLKIDIHVIIPYEGLLGDQMGQIEKIFKVVYPVLEHHHHHH

SEQ ID NO: 175 - protein, 251 aa, synthetic construct
MAQVQLVESGGGLVQAGGSLRLSCAASGSFFMSNVMAWYRQAPGKARELIAAIRGGDMSTVYDDSVKGRFTITRDDDKNILYLQMNDLKPEDTAMYYCKASGSSWGQGTQVTVSSAAAGEMETSQNPGEEKPQASPEGRPESETSCLVTTTDNQISTEQGFTLEDFVGDWRQTAGRNLDQVLEQGGVSSLFQNLGVSVTPIQRIVKSGENGLKIDIHVIIPYEGLLGDQMGQIEKIFKVVYPVLEHHHHHH